(19) United States
(12) Patent Application Publication
(10) Pub. No.: US 2025/0288311 A1
(43) Pub. Date: Sep. 18, 2025

(54) SURGICAL INSTRUMENT

(71) Applicant: LIVSMED INC., Seongnam-si (KR)

(72) Inventor: Woo Jung CHOI, Siheung-si (KR)

(21) Appl. No.: 19/082,699

(22) Filed: Mar. 18, 2025

(30) Foreign Application Priority Data
Mar. 18, 2024 (KR) 10-2024-0037460

(51) Int. Cl. A61B 17/29; A61B 17/00

(52) U.S. Cl. CPC A61B 17/29; A61B 2017/00398; A61B 2017/2929

(57) ABSTRACT

A surgical instrument mounted on a robot arm or manually operable for use in laparoscopic surgery or various surgeries, the surgical instrument includes an end tool including a first jaw and a second jaw, each rotatably formed and each configured to rotate in at least one direction, a manipulation portion configured to receive a signal input by a user to control an operation of the end tool, and a power generation portion disposed to be at least partially accommodated in a housing of the manipulation portion and includes a motor pack having at least one motor configured to generate power to drive the end tool based on a signal input to the manipulation portion, and a roll drive motor configured to generate driving force to roll-rotate the motor pack.

### CROSS-REFERENCE TO RELATED APPLICATION

This application is based on and claims priority under 35 U.S.C. § 119 to Korean Patent Application No. 10-2024-0037460, filed on Mar. 18, 2024, in the Korean Intellectual Property Office, the disclosure of which is incorporated by reference herein in its entirety.

### BACKGROUND

### 1. Field

The present disclosure relates to a surgical instrument, and more particularly, to a surgical instrument that may be mounted on a robot arm or is manually operable for use in laparoscopic surgery or various surgeries.

### 2. Description of the Related Art

Surgery denotes a process of curing illness by cutting, incising, or manipulating the skin, the mucosa layer, and other tissues by using a medical instrument. In particular, open surgery in which the skin of the surgical site is incised and opened to treat, shape, or remove organs, etc. therein and the like causes problems such as bleeding, side effects, patient pain, and scarring. Therefore, recently, surgery performed by inserting only a medical device, for example, laparoscopic surgical instrument, microsurgical microscope, etc., by forming a predetermined hole in the skin or surgery using a robot has been spotlighted as an alternative.

Here, a surgical robot refers to a robot having a function capable of replacing a surgical operation that has been performed by a surgeon. Compared to humans, such a surgical robot has the advantage of being able to perform more accurate and precise movements and enable remote surgeries.

Surgical robots currently being developed around the world include bone surgical robots, laparoscopic surgical robots, and stereotactic surgical robots. Among these, a laparoscopic surgical robot is a robot that performs minimally invasive surgeries by using a laparoscope and small surgical tools.

Laparoscopic surgery is a cutting-edge surgical technique that involves forming one or more small holes in the stomach, inserting a laparoscope, which is an endoscope to look inside the stomach, and then performing a surgery, and is a field in which a significant development is expected in the future. Recently, laparoscopes are equipped with computer chips, so they can obtain clearer and enlarged images than those seen with the naked eye, and are also developed to the point where any type of surgery is possible by using specially designed laparoscopic surgical instruments while viewing the screen through a monitor.

Moreover, the laparoscopic surgery has the advantages of having almost the same surgical scope as an open surgery while having fewer complications, allowing treatments to be started much earlier after the surgery, and having an excellent ability to maintain a patient's physical strength and immune function. As a result, the laparoscopic surgery is gradually being recognized as a standard surgery in the treatment of the colon cancer in the United States and Europe.

Meanwhile, a surgical robot generally includes a master robot and a slave robot. When an operator manipulates a control lever (e.g., a handle) provided on the master robot, a surgical tool coupled to a robot arm of the slave robot or held by the robot arm is manipulated to perform a surgery.

The above-mentioned background art is technical information that the inventor has possessed for the derivation of the present disclosure or acquired in the process of derivation of the present disclosure, and cannot necessarily be said to be a known technique disclosed to the general public prior to the filing of the present disclosure.

### SUMMARY

The present disclosure relates to a surgical instrument capable of axial rotation (roll) without limitation in rotation angle.

However, these effects are exemplary, and the effects of the present disclosure are not limited thereto.

Additional aspects will be set forth in part in the description which follows and, in part, will be apparent from the description, or may be learned by practice of the presented embodiments of the disclosure.

In an embodiment of the present disclosure, a surgical instrument may include an end tool including a first jaw and a second jaw, each rotatably formed and each configured to rotate in at least one direction, a manipulation portion configured to receive a signal input by a user to control an operation of the end tool, and a power generation portion disposed to be at least partially accommodated in a housing of the manipulation portion and includes a motor pack having at least one motor configured to generate power to drive the end tool based on a signal input to the manipulation portion, and a roll drive motor configured to generate driving force to roll-rotate the motor pack.

In another embodiment, the motor pack may be configured to rotate independently of a movement of the housing.

In the other embodiment, the roll drive motor may be provided in the motor pack.

In the other embodiment, the power generation portion may include a first gear formed to have a circular shape and having gear teeth formed on an inner circumferential surface, and a second gear disposed on one side of the roll drive motor and engaged with the first gear.

In the other embodiment, the first gear may be fixed to the inner circumferential surface of the housing, and the second gear may be formed to move along the inner circumferential surface of the first gear while being rotated by driving the roll drive motor.

In the other embodiment, the end tool may be connected to the motor pack and be configured to roll-rotate together with the motor pack.

In the other embodiment, the surgical instrument may further include a power transmission portion having at least one wire transmitting power generated by the power generation portion to the end tool.

In the other embodiment, the power transmission portion may be connected to the motor pack and be configured to roll-rotate together with the motor pack.

In the other embodiment, the surgical instrument may further include a connection portion disposed between the power transmission portion and the end tool and accommodating the wire therein.

In the other embodiment, the connection portion may be connected to the power transmission portion and be configured to roll-rotate together with the power transmission portion by roll rotation of the power transmission portion.

In the other embodiment, the power generation portion may further include a pulley coupling plate coupled to the power transmission portion, and the pulley coupling plate may be configured to rotate together with the motor pack by driving of the roll drive motor.

In the other embodiment, the power transmission portion may be detachably coupled to the pulley coupling plate.

In the other embodiment, the power transmission portion may be configured to roll-rotate together with the pulley coupling plate while being coupled to the pulley coupling plate.

In the other embodiment, the motor pack may further include a yaw drive motor configured to generate power to yaw-rotate the end tool, and a pitch drive motor configured to generate power to pitch-rotate the end tool.

In the other embodiment, the roll drive motor, the yaw drive motor, and the pitch drive motor may be driven independently of one another and independently perform yaw rotation of the end tool, pitch rotation of the end tool, and roll rotation of the motor pack.

In the other embodiment, the power generation portion may further include a base plate connected to the roll drive motor, the yaw drive motor, and the pitch drive motor, and the base plate may be rotated by driving the roll drive motor, and the roll drive motor, the yaw drive motor, and the pitch drive motor may rotate simultaneously by the rotation of the base plate.

In the other embodiment, the end tool may further include a moving member configured to move along a lengthwise direction of the end tool, and the motor pack may further include a firing drive motor configured to generate power to linearly move the moving member.

In the other embodiment, the roll drive motor, the yaw drive motor, the pitch drive motor, and the firing drive motor may be driven independently of one another and independently perform yaw rotation of the end tool, pitch rotation of the end tool, roll rotation of the motor pack, and linear movement of the moving member.

In the other embodiment, the power generation portion may further include a base plate connected to the roll drive motor, the yaw drive motor, the pitch drive motor, and the firing drive motor, and the base plate may be rotated by driving the roll drive motor, and the roll drive motor, the yaw drive motor, the pitch drive motor, and the firing drive motor may rotate simultaneously by the rotation of the base plate.

In the other embodiment, the power generation portion may further include a bearing plate connected to the base plate, and first bearings arranged to cover an outer circumferential surface of the bearing plate.

In the other embodiment, the surgical instrument may further include a circuit unit disposed on one side of the motor pack and configured to control driving of the motor pack.

In the other embodiment, the circuit unit may be connected to the motor pack and be configured to roll-rotate together with the motor pack.

In the other embodiment, the surgical instrument may further include a slip ring disposed on one side of the circuit unit and electrically connecting the motor pack and the circuit unit to each other.

In the other embodiment, the surgical instrument may further include a sub-circuit unit configured to pre-process a signal for controlling the motor pack and transmit a pre-processed signal to the circuit unit.

In the other embodiment, the sub-circuit unit may be disposed on one side of the manipulation portion and configured not to rotate even when the motor pack and the circuit unit roll-rotate.

In the other embodiment, the surgical instrument may further include at least one encoder configured to measure a rotation angle of the motor pack.

The present disclosure also relates to a driving module for a surgical instrument, the driving module includes an end tool including a pair of jaws each rotatably formed and configured to rotate in at least one direction, a power generation portion having a motor pack, which includes at least one motor configured to generate power to drive the end tool and mechanically connected to the end tool configured to roll-rotate together with the end tool, and a roll drive motor configured to generate driving force to roll-rotate the end tool and the motor pack together, and a module body including a housing accommodating at least a portion of the power generation portion.

In the other embodiment of the present disclosure, the module body may be formed to be mountable on a surgical robot or a manipulation portion manipulable by a user.

In the other embodiment, the motor pack may be configured to rotate independently of a movement of the housing.

In the other embodiment, the roll drive motor may be provided in the motor pack.

In the other embodiment, the power generation portion may include a first gear formed to have a circular shape and having gear teeth formed on an inner circumferential surface, and a second gear disposed on one side of the roll drive motor and engaged with the first gear.

In the other embodiment, the first gear may be fixed to the inner circumferential surface of the housing, and the second gear may be formed to move along the inner circumferential surface of the first gear while being rotated by driving the roll drive motor.

In the other embodiment, the motor pack may further include a yaw drive motor configured to generate power to yaw-rotate the end tool, and a pitch drive motor configured to generate power to pitch-rotate the end tool.

In the other embodiment, the roll drive motor, the yaw drive motor, and the pitch drive motor may be driven independently of one another and independently perform yaw rotation of the end tool, pitch rotation of the end tool, and roll rotation of the motor pack.

In the other embodiment, the end tool may further include a moving member configured to move along a lengthwise direction of the end tool, and the motor pack may further include a firing drive motor configured to generate power to linearly move the moving member.

In the other embodiment, the roll drive motor, the yaw drive motor, the pitch drive motor, and the firing drive motor may be driven independently of one another and independently perform yaw rotation of the end tool, pitch rotation of the end tool, roll rotation of the motor pack, and linear movement of the moving member.

### BRIEF DESCRIPTION OF THE DRAWINGS

The aforementioned and other aspects, features, and advantages of certain embodiments of the present disclosure will be more apparent from the following description taken in conjunction with the accompanying drawings, in which:

FIG. 1 is a perspective view of a surgical instrument according to an embodiment of the present disclosure.

FIG. 2 is a side view of the surgical instrument of FIG. 1.

FIG. 3 is a perspective view of an end tool according to an embodiment of the present disclosure.

FIG. 4 is a perspective view of the end tool of FIG. 3 with an end tool hub and a pitch hub removed.

FIG. 5 is a diagram for describing the internal structure of a power transmission portion according to an embodiment of the present disclosure.

FIG. 6 is a rear view of the power transmission portion of FIG. 5.

FIG. 7 is a diagram for describing the arrangement of pulleys and wires of the power transmission portion of FIG. 5.

FIG. 8 is a diagram showing a manipulation portion and a power generation portion according to an embodiment of the present disclosure.

FIG. 9 is a perspective view of the power generation portion of FIG. 8.

FIG. 10 is a rear view of the power generation portion of FIG. 9.

FIG. 11 is a diagram for describing the gear structure of the power generation portion of FIG. 9.

FIG. 12 is a front view of the structure of FIG. 11.

FIG. 13 is a diagram for describing the rotation of the power generation portion of FIG. 9.

FIG. 14 is a perspective view of an end tool according to another embodiment of the present disclosure.

FIG. 15 is a perspective view of the end tool of FIG. 14 with an end tool hub and a pitch hub removed.

FIG. 16 is a perspective view of a first jaw and a cartridge of the end tool of FIG. 14.

FIG. 17 is an exploded perspective view of the cartridge of FIG. 16.

FIG. 18 is a perspective cross-sectional view for describing the internal structure of the cartridge of FIG. 16.

FIG. 19 is a schematic perspective view of an end tool according to another embodiment of the present disclosure.

FIG. 20 is a perspective view of the end tool of FIG. 19, viewed from another direction.

FIG. 21 is a schematic perspective view of the end tool of FIG. 19 with a second jaw removed.

FIG. 22 is a schematic perspective view of the end tool of FIG. 21 with a cartridge removed.

FIG. 23 is a transparent perspective view of the structure of FIG. 22.

FIG. 24 is a diagram for describing the internal structure of a power transmission portion according to the other embodiment of the present disclosure.

FIG. 25 is a rear view of the power transmission portion of FIG. 24.

FIG. 26 is a diagram for describing the arrangement of pulleys and wires of the power transmission portion of FIG. 24.

FIG. 27 is a diagram showing a manipulation portion and a power generation portion according to the other embodiment of the present disclosure.

FIG. 28 is a perspective view of the power generation portion of FIG. 27.

FIG. 29 is a rear view of the power transmission portion of FIG. 28.

FIG. 30 is a diagram for describing the gear structure of the power generation portion of FIG. 28.

FIG. 31 is a front view of the structure of FIG. 30.

FIG. 32 is a diagram for describing the rotation of the power generation portion of FIG. 28.

FIG. 33 is a diagram for describing the roll operation of a surgical instrument according to the other embodiment of the present disclosure.

FIGS. 34 and 35 are diagrams for describing the coupling structure of a surgical instrument according to the other embodiment of the present disclosure.

FIG. 36 is a diagram showing the internal structure of a surgical instrument according to the other embodiment of the present disclosure.

FIGS. 37 to 41 are diagrams showing the pitch rotation motion of a surgical instrument according to the other embodiment of the present disclosure.

FIGS. 42 to 46 are diagrams showing the yaw rotation motion of a surgical instrument according to the other embodiment of the present disclosure.

FIGS. 47 to 51 are diagrams showing states that a surgical instrument according to the other embodiment of the present disclosure has pitch-rotated and yaw-rotated.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain various aspects. As used herein, the term “and/or” includes any and all combinations of one or more of the associated listed items. Expressions such as “at least one of,” when preceding a list of elements, modify the entire list of elements and do not modify the individual elements of the list.

The present disclosure may include various embodiments and modifications, and embodiments thereof will be illustrated in the drawings and will be described herein in detail. Effects and features of the present disclosure and the methods for achieving them will become apparent from the detailed description of the embodiments provided below along with the drawings. However, the present disclosure is not limited to embodiments disclosed below, but may be implemented in various forms.

Reference will now be made in detail to embodiments, examples of which are illustrated in the accompanying drawings. In the drawings, the same elements are denoted by the same reference numerals, and a repeated explanation thereof will not be given.

It will be understood that although the terms “first”, “second”, etc. may be used herein to describe various elements, these elements should not be limited by these terms. These elements are only used to distinguish one element from another.

As used herein, the singular forms “a,” “an” and “the” are intended to include the plural forms as well, unless the context clearly indicates otherwise.

It will be further understood that the terms “comprises” and/or “comprising” used herein specify the presence of stated features or components, but do not preclude the presence or addition of one or more other features or components.

Sizes of elements in the drawings may be exaggerated for convenience of explanation. In other words, since sizes and thicknesses of components in the drawings are arbitrarily illustrated for convenience of explanation, the following embodiments are not limited thereto.

In the following examples, the x-axis, the y-axis and the z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

When a certain embodiment may be implemented differently, a specific process order may be performed differently from the described order. For example, two consecutively described processes may be performed substantially at the same time or performed in the reverse order of the described order.

Hereinafter, a surgical instrument according to the present disclosure will be described in detail with reference to the drawings based on the above-described principles.

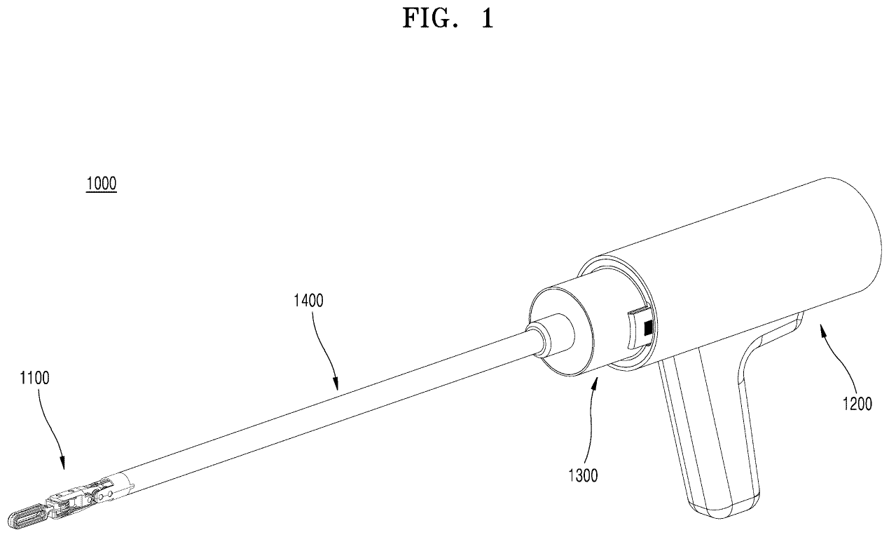

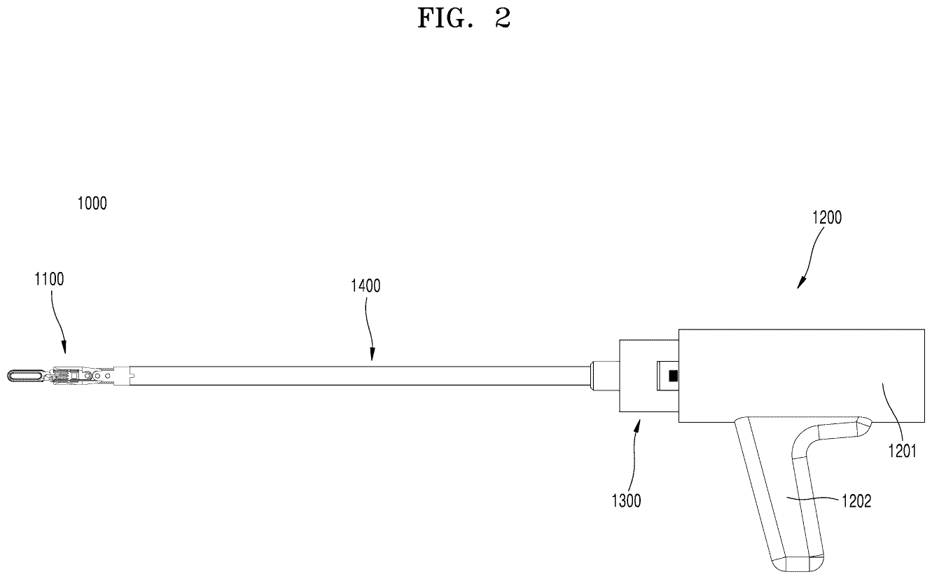

FIG. 1 is a perspective view of a surgical instrument 1000 according to an embodiment of the present disclosure, and FIG. 2 is a side view of the surgical instrument 1000 of FIG. 1 viewed in a lateral direction.

Referring to FIGS. 1 and 2, a surgical instrument 1000 according to an embodiment of the present disclosure may include an end tool 1100, a manipulation portion 1200, a power transmission portion 1300, and a connection portion 1400.

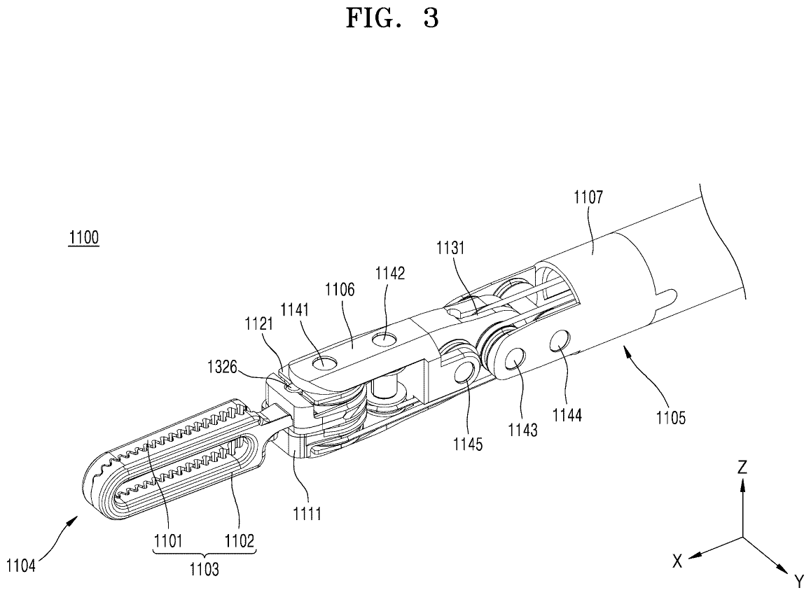

The end tool 1100 may be formed at a first end of the connection portion 1400 and is inserted into a surgical site to perform an operation necessary for surgery. As an example of such the end tool 1100, a pair of jaws 1103 for performing a grip motion as shown in FIG. 3 may be used. The end tool 1100 may be connected to the manipulation portion 1200, the power transmission portion 1300, and the connection portion 1400 to be described later and may receive the driving force of the manipulation portion 1200 through the power transmission portion 1300 and/or the connection portion 1400, thereby performing operations needed for surgery, such as gripping, cutting, and suturing. However, the present disclosure is not limited thereto, and various devices for surgery may be used as the end tool 1100. For example, hereinafter, for convenience of explanation, the end tool 1100 used as a surgical clamp and an end tool 3100 used as a stapler will be described as examples. However, the present disclosure is not limited thereto, and components such as a surgical grasper, a vessel sealer, and a one-armed cauterizer may also be used as end tools.

A user may operate the end tool 1100 by manipulating the manipulation portion 1200. For example, the manipulation portion 1200 may be a component to allow the user to input signals to control the operation of the end tool 1100. In other words, the manipulation portion 1200 may also be considered as a component that receives signals for controlling the operation of the end tool 1100 from the user. Here, a signal for controlling the operation of the end tool 1100 may be a mechanical manipulation such as pressing a button or a switch, a mechanical manipulation such as rotation or movement of a particular member, and an electrical signal generated by such a mechanical manipulation. However, the present disclosure is not limited thereto. The manipulation portion 1200 may be provided in the form of an interface that may be directly manipulated by a doctor, e.g., a forceps-like shape, a stick-like shape, a lever-like shape, etc., and, when the doctor manipulates the manipulation portion 1200, the end tool 1100 connected to the corresponding interface and inserted into the body of a patient performs a certain operation, thereby performing a surgery. Here, although FIG. 1 shows that the manipulation portion 1200 may be formed in the shape of a gun, the present disclosure is not limited thereto, and the manipulation portion 1200 may be in various forms to be connected to and manipulate the end tool 1100.

For example, the manipulation portion 1200 may be a separate part or a separate module that is separated from a driving module of the surgical instrument. Here, the driving module of the surgical instrument may refer to a part or a module of the surgical instrument including the end tool 1100, the power transmission portion 1300, the connection portion 1400, and a power generation portion 1500.

In detail, for example, the driving module of the surgical instrument may be a separate component that may be mounted on the manipulation portion 1200 that is directly operable by a user. For example, the driving module of the surgical instrument may be formed to be detachable from the manipulation portion 1200. In this case, the driving module of the surgical instrument may include a module body, and the module body may include a coupling structure to be coupled to the manipulation portion 1200.

In this way, when the driving module of the surgical instrument is formed to be detachable from the manipulation portion 1200, a user may easily replace the driving module of the surgical instrument as needed.

In another example, the manipulation portion 1200 may be replaced by a surgical robot. In other words, the driving module of the surgical instrument may be a separate component that may be mounted on a surgical robot.

A surgical robot may refer to a robot that may perform surgeries or procedures by being manipulated by a user (e.g., a surgeon).

In detail, for example, a surgical robot may include a master robot and a slave robot.

The master robot may include manipulation members that a user may hold and manipulate in both hands and a display member that displays images captured through a laparoscope.

The slave robot may include one or more robot arm units. Here, each robot arm unit may be provided in the form of a module that may operate independently from each other. Here, two or more robot arm units may be equipped with driving modules for surgical instruments. For example, a driving module for a surgical instrument may be formed to be attachable to and detachable from a surgical robot (in detail, a slave robot). In this case, the driving module of the surgical instrument may include a module body, and the module body may include a coupling structure to be coupled to a surgical robot.

In this way, when the driving module of the surgical instrument is formed to be detachable from the surgical robot, a user may easily replace the driving module of the surgical instrument as needed.

Hereinafter, for convenience of explanation, the technical idea of the present disclosure will be described in detail based on a surgical instrument including the manipulation portion 1200 as an example. However, one of ordinary skill in the art will understand that a surgical instrument may be implemented as a driving module for a surgical instrument that is formed to be mountable on a surgical robot or a manipulation portion directly operable by a user.

The power transmission portion 1300 may be formed at a second end of the connection portion 1400 and may serve to transmit power generated by the power generation portion 1500, which will be described later, to the end tool 1100. For example, the power transmission portion 1300 may be disposed between the end tool 1100 and the manipulation portion 1200. As described later, when a user such as a doctor manipulates the manipulation portion 1200, the power generation portion 1500 generates power to control the end tool 1100, and generated power may be transmitted to the end tool 1100 through the power transmission portion 1300. The power transmission portion 1300 may include a plurality of wires, pulleys, links, nodes, gears, etc.

The connection portion 1400 may be formed in the shape of a hollow shaft, and one or more wires and cables may be accommodated therein. The manipulation portion 1200 may be coupled to the first end of the connection portion 1400, the power transmission portion 1300 may be coupled to the second end of the connection portion 1400, and the power transmission portion 1300 may be connected to the manipulation portion 1200. In other words, it may be said that the connection portion 1400 serves to connect the manipulation portion 1200 and the end tool 1100.

Meanwhile, a connector (not shown) may be formed at the manipulation portion 1200. The connector (not shown) may be connected to an external power source (not shown), and the connector (not shown) may be connected to the end tool 1100 through electric wires and transmit electrical energy supplied from the external power source (not shown) to the end tool 1100. Also, the electrical energy transmitted to the end tool 1100 in this way may provide driving force to perform a yaw rotation operation, a pitch rotation operation, an actuation operation, a staple operation, etc. of the end tool 1100, which will be described later. Alternatively, the electrical energy transmitted to the end tool 1100 may provide driving force for performing cutting and cauterizing functions of the end tool 1100, such as a monopolar method, a bipolar method, or an ultrasonic blade. Also, the electrical energy may be supplied to drive the power transmission portion 1300. Of course, it is also possible to use a built-in battery.

The manipulation portion 1200 may include a housing 1201 that forms the outer shape of the manipulation portion 1200. At least a portion of the power generation portion 1500 that generates power to control the end tool 1100 may be accommodated inside the housing 1201, as will be described later. Also, a circuit unit for controlling the operation of the power generation portion 1500 and a slip ring for supplying electrical energy to the power generation portion 1500, connecting communications, or transmitting various other signals may be accommodated inside the housing 1201.

A handle 1202 may be formed at the manipulation portion 1200. The handle 1202 may be a portion to be held by a user. Therefore, a user may use the surgical instrument 1000 according to the present disclosure while holding the handle 1202 of the manipulation portion 1200.

Meanwhile, although not shown in the drawing, buttons, switches, levers, etc. for controlling various operations of the end tool 1100 may be further formed at the manipulation portion 1200.

Hereinafter, the end tool 1100, the manipulation portion 1200, the power transmission portion 1300, the power generation portion 1500, etc. of the surgical instrument of FIGS. 1 and 2 will be described in more detail.

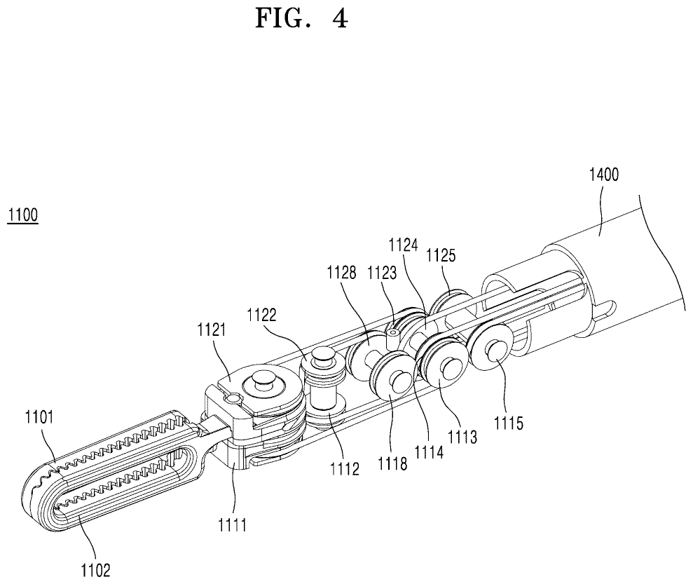

FIG. 3 is a perspective view of an end tool 1100 according to an embodiment of the present disclosure, and FIG. 4 is a perspective view of the end tool 1100 of FIG. 3 with an end tool hub 1106 and a pitch hub 1107 removed.

Referring to FIGS. 3 and 4, an end tool 1100 of a surgical instrument 1000 may include a first jaw 1101 and a second jaw 1102 that are rotatably formed.

In other words, the end tool 1100 of the surgical instrument 1000 according to an embodiment of the present disclosure may include a pair of jaws for performing a grip operation, that is, a first jaw 1101 and a second jaw 1102. Here, the first jaw 1101, the second jaw 1102, or components including the first jaw 1101 and the second jaw 1102 may be referred to as two jaws 1103.

Here, the end tool 1100 of the surgical instrument 1000 according to an embodiment of the present disclosure may be formed to be rotatable in at least one direction. For example, the end tool 1100 may be formed to perform a pitch motion around the Y-axis of FIG. 3 and, at the same time, perform a yaw motion and an actuation motion around the Z-axis of FIG. 3.

Here, a pitch motion, a yaw motion, and an actuation motion used in the present disclosure are each defined as follows.

First, a pitch motion refers to a motion that the end tool 1100 rotates in the vertical direction with respect to the extension direction (X-axis direction in FIG. 3) of the connection portion 1400, that is, a motion of rotating around the Y-axis of FIG. 3. In other words, the pitch motion refers to a motion that the end tool 1100 extending from the connection portion 1400 in the extension direction of the connection portion 1400 (X-axis direction in FIG. 3) rotates in the vertical direction around the Y-axis with respect to the connection portion 1400.

Next, a yaw motion refers to a motion that the end tool 1100 rotates in the horizontal direction with respect to the extension direction (X-axis direction in FIG. 3) of the connection portion 1400, that is, a motion of rotating around the Z-axis of FIG. 3. In other words, the pitch motion refers to a motion that the end tool 1100 extending from the connection portion 1400 in the extension direction of the connection portion 1400 (X-axis direction in FIG. 3) rotates in the horizontal direction around the Z-axis with respect to the connection portion 1400. In other words, the yaw motion refers to a motion that the two jaws 1103 formed at the end tool 1100 rotate in the same direction around the Z-axis.

Meanwhile, an actuation motion refers to a motion that, although the end tool 1100 rotates around the same rotation shaft as the yaw motion, the two jaws 1103 is closing or opening by rotating in directions opposite to each other. In other words, the actuation motion refers to a motion that the two jaws 1103 formed at the end tool 1100 rotate in opposite directions around the Z-axis.

The end tool 1100 may include a plurality of pulleys including a first pulley 1111 related to the rotational movement of the first jaw 1101. Also, the end tool 1100 may include a plurality of pulleys including a second pulley 1121 related to the rotational movement of the second jaw 1102.

Here, the drawings show that the pulleys facing each other are formed in parallel to each other. However, the present disclosure is not limited thereto, and pulleys may be formed at various positions in various sizes suitable for the configuration of the end tool 1100.

Also, the end tool 1100 according to the present embodiment may include an end tool hub 1106 and a pitch hub 1107.

A first rotation shaft 1141 and a second rotation shaft 1142 are inserted through the end tool hub 1106, and the end tool hub 1106 may also accommodate at least portions of one or more pulleys axially coupled to the first rotation shaft 1141 therein. Also, the end tool hub 1106 may accommodate at least portions of one or more pulleys axially coupled to the second rotation shaft 1142 therein.

Meanwhile, a third pulley 1131 serving as an end tool pitch pulley may be formed at one end of the end tool hub 1106. The third pulley 1131 may be formed as one body with the end tool hub 1106. In other words, a pulley having a disk-like shape may be formed at one end of the end tool hub 1106, and a groove in which a wire may be wound may be formed on the outer circumferential surface of the pulley. Alternatively, the third pulley 1131 may be formed as a member separate from the end tool hub 1106 and coupled to the end tool hub 1106.

A third rotation shaft 1143 and a fourth rotation shaft 1144 are inserted through the pitch hub 1107, and the pitch hub 1107 may be axially coupled to the end tool hub 1106 (and the third pulley 1131) by the third rotation shaft 1143. Therefore, the end tool hub 1106 and the third pulley 1131 may be formed to be rotatable with respect to the pitch hub 1107 around the third rotation shaft 1143.

Also, the pitch hub 1107 may accommodate at least portions of one or more pulleys axially coupled to the third rotation shaft 1143 therein. Also, the pitch hub 1107 may accommodate at least portions of one or more pulleys axially coupled to the fourth rotation shaft 1144 therein.

Also, the end tool 1100 according to the present disclosure may include the first rotation shaft 1141, the second rotation shaft 1142, the third rotation shaft 1143, and the fourth rotation shaft 1144. As described above, the first rotation shaft 1141 and the second rotation shaft 1142 may be inserted through the end tool hub 1106, and the third rotation shaft 1143 and the fourth rotation shaft 1144 may be inserted through the pitch hub 1107. Also, according to an optional embodiment, the end tool 1100 may further include a fifth rotation shaft 1145.

The first rotation shaft 1141, the second rotation shaft 1142, the third rotation shaft 1143, the fourth rotation shaft 1144, and the fifth rotation shaft 1145 may be sequentially arranged in a direction from a distal end 1104 of the end tool 1100 to a proximal end 1105 of the end tool 1100. Therefore, sequentially from the distal end 1104, the first rotation shaft 1141 may be referred to as a first pin, the second rotation shaft 1142 may be referred to as a second pin, the fifth rotation shaft 1145 may be referred to as a 2.5-th pin, the third rotation shaft 1143 may be referred to as a third pin, and the fourth rotation shaft 1144 may be referred to as a fourth pin.

Here, the first rotation shaft 1141 may function as an end tool jaw pulley rotation shaft, the second rotation shaft 1142 may function as an end tool jaw auxiliary pulley rotation shaft, the third rotation shaft 1143 may function as an end tool pitch rotation shaft, and the fourth rotation shaft 1144 may function as an end tool pitch auxiliary rotation shaft of the end tool 1100. Also, the fifth rotation shaft 1145 may function as a pitch extra rotation shaft of the end tool 1100.

One or more pulleys may be fitted to each of the first rotation shaft 1141, the second rotation shaft 1142, the third rotation shaft 1143, and the fourth rotation shaft 1144.

The first pulley 1111 functions as an end tool first jaw pulley, and the second pulley 1121 functions as an end tool second jaw pulley. The first pulley 1111 may be referred to as a first jaw pulley, the second pulley 1121 may be referred to as a second jaw pulley, and the two components may be collectively referred to as end tool jaw pulleys or simply jaw pulleys.

The first pulley 1111 and the second pulley 1121, which are end tool jaw pulleys, may be formed to face each other and may be formed to be rotatable independently of each other around the first rotation shaft 1141, which is an end tool jaw pulley rotation shaft.

Here, although the drawings show that the first pulley 1111 and the second pulley 1121 may be formed to rotate around a single rotation shaft (e.g., the first rotation shaft 1141), end tool jaw pulleys may also be formed to be rotatable around separate rotation shafts. Here, the first jaw 1101 may be fixedly coupled to the first pulley 1111 and rotate together with the first pulley 1111, and the second jaw 1102 may be fixedly coupled to the second pulley 1121 and rotate together with the second pulley 1121. A yaw motion and an actuation motion of the end tool 1100 may be performed according to rotations of the first pulley 1111 and the second pulley 1121. In other words, a yaw motion may be performed when the first pulley 1111 and the second pulley 1121 rotate in the same direction around the first rotation shaft 1141, and an actuation motion is performed when the first pulley 1111 and the second pulley 1121 rotate in directions opposite to each other around the first rotation shaft 1141.

Here, the first jaw 1101 and the first pulley 1111 may be formed as separate members and coupled to each other, or the first jaw 1101 and the first pulley 1111 may be formed as one body. In the same regard, the second jaw 1102 and the second pulley 1121 may be formed as separate members and coupled to each other, or the second jaw 1102 and the second pulley 1121 may be formed as one body.

Also, one or more auxiliary pulleys may be arranged adjacent to the first pulley 1111 and second pulley 1121.

These pulleys may be formed, such that one or more wires are wound around each of the pulleys, the pulleys may be rotated by wires, and the wires may move along the pulleys. Therefore, power may be transmitted to the end tool 1100.

Meanwhile, the end tool 1100 described above is an embodiment of an end tool that may be mounted on the surgical instrument 1000 according to the present disclosure, and the technical idea of the present disclosure is not limited thereto. Some components may be changed, omitted, or added as needed.

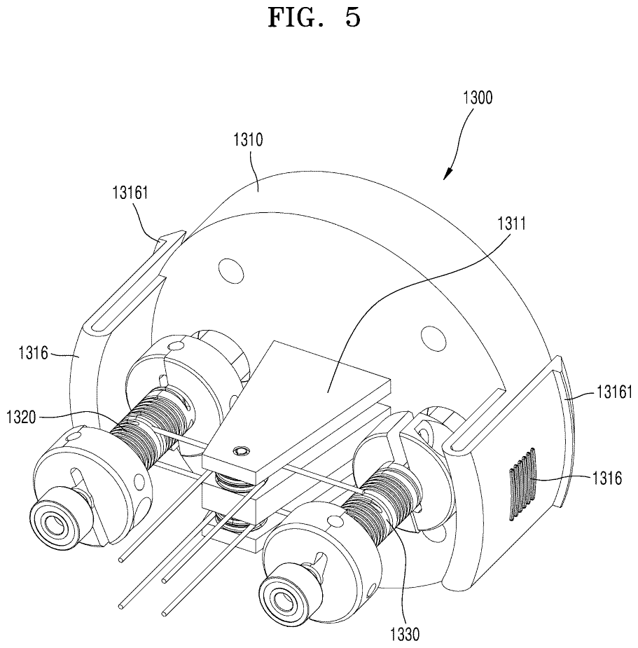

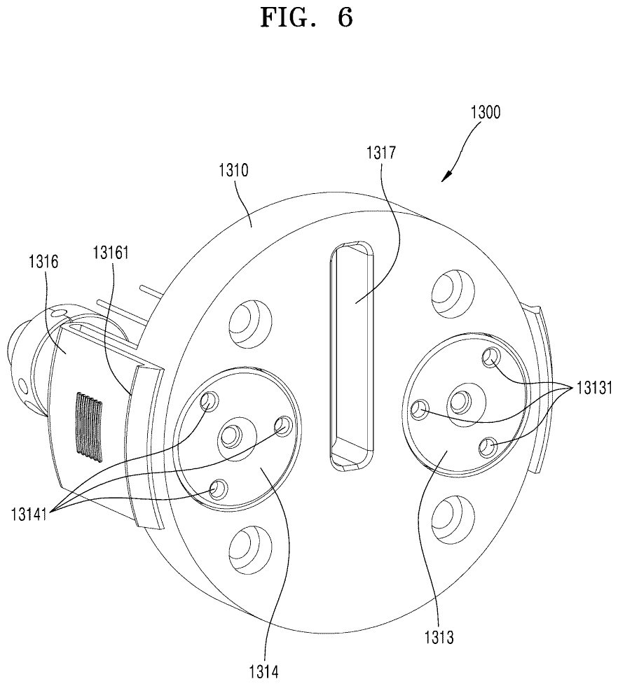

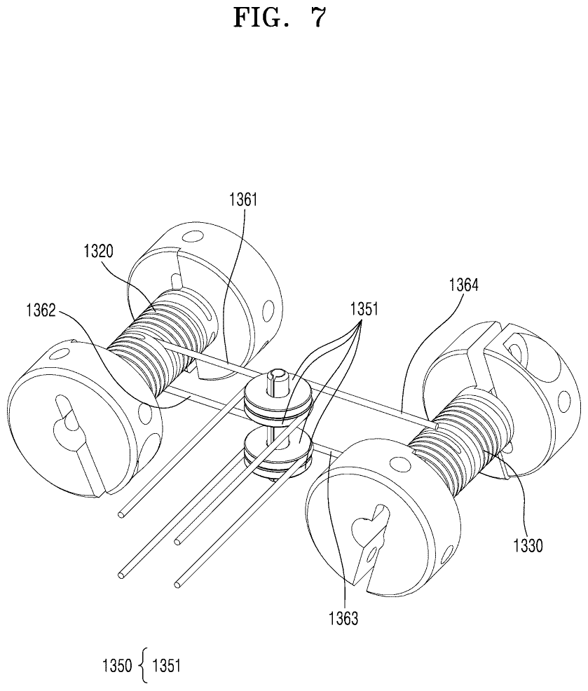

FIG. 5 is a diagram for describing the internal structure of a power transmission portion 1300 according to an embodiment of the present disclosure, FIG. 6 is a rear view of the power transmission portion 1300 of FIG. 5, and FIG. 7 is a diagram for describing the arrangement of pulleys and wires of the power transmission portion 1300 of FIG. 5.

Referring to FIGS. 5 to 7, the power transmission portion 1300 according to an embodiment of the present disclosure may include a pulley frame 1310, at least one pulley, and at least one wire.

The pulley frame 1310 may form the overall framework of the power transmission portion 1300.

At least one pulley may be disposed in the pulley frame 1310. Here, disposing a pulley should be interpreted in a broad sense. For example, disposing a pulley may mean that the pulley is directly connected to the pulley frame 1310 or may mean that a rotation shaft is installed in the pulley frame 1310 and the pulley is connected to the rotation shaft. Alternatively, disposing a pulley may mean that a separate member is provided in the pulley frame 1310, a rotation shaft is connected to the separate member, and the pulley is connected to the rotation shaft. Alternatively, disposing a pulley may mean that a hole is formed in the pulley frame 1310, a rotation shaft is disposed to pass through the hole, and a pulley is installed on the rotation shaft.

The power transmission portion 1300 may include at least one pulley.

A pulley is a member around which a wire is wound, and a groove in which a wire may be wound may be formed on the pulley.

According to an embodiment, the power transmission portion 1300 may include a yaw pulley 1320 and a pitch pulley 1330. Also, the power transmission portion 1300 may include at least one yaw wire and at least one pitch wire.

The yaw pulley 1320 is a pulley associated with the yaw rotation of the end tool 1100, and the first yaw wire 1361 and the second yaw wire 1362 are wires associated with the yaw rotation of the end tool 1100.

The yaw pulley 1320 may be disposed in one region of the pulley frame 1310.

The first yaw wire 1361 and the second yaw wire 1362 may be arranged at the end tool 1100 and extend from a pulley associated with the yaw rotation of the end tool 1100 toward the yaw pulley 1320.

According to an embodiment, the first yaw wire 1361 and the second yaw wire 1362 may be a pair of yaw wires. As described later, the first yaw wire 1361 and the second yaw wire 1362 may be connected to the upper portion and the lower portion of the yaw pulley 1320, respectively.

According to a specific embodiment, the at least one yaw wire may include a first yaw wire 1361 and a second yaw wire 1362. In FIG. 7, the first yaw wire 1361 may extend to the upper portion of the yaw pulley 1320 and be connected to the yaw pulley 1320. In FIG. 7, the second yaw wire 1362 may extend to the lower portion of the yaw pulley 1320 and be connected to the yaw pulley 1320.

After the first yaw wire 1361 and the second yaw wire 1362 are connected to the yaw pulley 1320, the first yaw wire 1361 and the second yaw wire 1362 may be wound around or unwound from the yaw pulley 1320 when the yaw pulley 1320 rotates. In other words, when the yaw pulley 1320 rotates in one direction, one yaw wire is wound around the yaw pulley 1320, and the other one yaw wire is unwound from the yaw pulley 1320. For example, when the yaw pulley 1320 rotates clockwise in FIG. 7, the first yaw wire 1361 is unwound from the yaw pulley 1320, and the second yaw wire 1362 is wound around the yaw pulley 1320. On the contrary, when the yaw pulley 1320 rotates counterclockwise in FIG. 7, the first yaw wire 1361 is wound around the yaw pulley 1320, and the second yaw wire 1362 is unwound from the yaw pulley 1320.

In other words, it may be said that, when the yaw pulley 1320 rotates, the pair of the first yaw wire 1361 and the second yaw wire 1362 may move in opposite directions around the yaw pulley 1320.

In this regard, when the yaw pulley 1320 rotates and moves the pair of the first yaw wire 1361 and the second yaw wire 1362 in different directions, a pulley connected to the end tool 1100 through the first yaw wire 1361 and the second yaw wire 1362 may rotate in a direction corresponding to the directions in which the pair of the first yaw wire 1361 and the second yaw wire 1362 are moved. Therefore, the pulley connected to the end tool 1100 through the first yaw wire 1361 and the second yaw wire 1362 may implement yaw rotation of the end tool 1100 while rotating in one direction.

The pitch pulley 1330 is a pulley associated with the pitch rotation of the end tool 1100, and the first pitch wire 1363 and the second pitch wire 1364 are wires associated with the pitch rotation of the end tool 1100.

The pitch pulley 1330 may be disposed in one region of the pulley frame 1310.

According to an embodiment, the pitch pulley 1330 may be disposed on a side opposite to the yaw pulley 1320. According to a preferred embodiment, the pitch pulley 1330 and the yaw pulley 1320 may be arranged symmetrically on both sides of the pulley frame 1310 around the center of the pulley frame 1310. Therefore, as described later, the first yaw wire 1361 and the second yaw wire 1362, and a first pitch wire 1363 and a second pitch wire 1364 extending through at least one auxiliary pulley 1350 may approach to the yaw pulley 1320 and the pitch pulley 1330 at almost vertical angles, respectively.

The first pitch wire 1363 and the second pitch wire 1364 may be arranged at the end tool 1100 and extend from a pulley associated with the pitch rotation of the end tool 1100 toward the pitch pulley 1330.

According to an embodiment, the first pitch wire 1363 and the second pitch wire 1364 may be a pair of pitch wires. As described later, the first pitch wire 1363 and the second pitch wire 1364 may be connected to the upper portion and the lower portion of the pitch pulley 1330, respectively.

As a specific example, the pitch wires may include a first pitch wire 1363 and a second pitch wire 1364. In FIG. 7, the first pitch wire 1363 may extend to the lower portion of the pitch pulley 1330 and be connected to the pitch pulley 1330. In FIG. 7, the second pitch wire 1364 may extend to the upper portion of the pitch pulley 1330 and be connected to the pitch pulley 1330.

After the first pitch wire 1363 and the second pitch wire 1364 are connected to the pitch pulley 1330, the first pitch wire 1363 and the second pitch wire 1364 may be wound around or unwound from the pitch pulley 1330 when the pitch pulley 1330 rotates. In other words, when the pitch pulley 1330 rotates in one direction, one pitch wire is wound around the pitch pulley 1330, and the other one pitch wire is unwound from the pitch pulley 1330. For example, when the pitch pulley 1330 rotates clockwise in FIG. 7, the first pitch wire 1363 is unwound from the pitch pulley 1330, and the second pitch wire 1364 is wound around the pitch pulley 1330. On the contrary, when the pitch pulley 1330 rotates counterclockwise in FIG. 7, the first pitch wire 1363 is wound around the pitch pulley 1330, and the second pitch wire 1364 is unwound from the pitch pulley 1330.

In other words, it may be said that, when the pitch pulley 1330 rotates, the pair of the first pitch wire 1363 and the second pitch wire 1364 may move in opposite directions around the pitch pulley 1330.

In this regard, when the pitch pulley 1330 rotates and moves the pair of the first pitch wire 1363 and the second pitch wire 1364 in different directions, a pulley connected to the end tool 1100 through the first pitch wire 1363 and the second pitch wire 1364 may rotate in a direction corresponding to the directions in which the pair of the first pitch wire 1363 and the second pitch wire 1364 are moved. Therefore, the pulley connected to the end tool 1100 through the first pitch wire 1363 and the second pitch wire 1364 may implement pitch rotation of the end tool 1100 while rotating in one direction.

According to the other embodiment, the power transmission portion 1300 may further include at least one auxiliary pulley 1350. For example, the at least one auxiliary pulley 1350 may include a first auxiliary pulley 1351 that is connected to the first yaw wire 1361 and the second yaw wire 1362, and the first pitch wire 1363 and the second pitch wire 1364, and changes paths of the first yaw wire 1361 and the second yaw wire 1362, and the first pitch wire 1363 and the second pitch wire 1364.

The first auxiliary pulley 1351 is disposed at the power transmission portion 1300 and may play a role of changing the paths of the first yaw wire 1361 and the second yaw wire 1362, and the first pitch wire 1363 and the second pitch wire 1364 extending from the end tool 1100 to the power transmission portion 1300.

An auxiliary pulley fixing portion 1311 may be formed at the pulley frame 1310. The auxiliary pulley fixing portion 1311 is a portion in which the first auxiliary pulley 1351 is installed. For example, the auxiliary pulley fixing portion 1311 may be formed as one body with the pulley frame 1310. Alternatively, the auxiliary pulley fixing portion 1311 may be formed as a separate member and may be coupled or assembled to the pulley frame 1310.

According to an embodiment, the auxiliary pulley fixing portion 1311 may include at least one through hole, and the rotation shaft of the first auxiliary pulley 1351 may be disposed to pass through the through hole. In other words, the first auxiliary pulley 1351 may be disposed to overlap the through hole formed in the auxiliary pulley fixing portion 1311, and the rotation shaft may be disposed to simultaneously penetrate through the first auxiliary pulley 1351 and the auxiliary pulley fixing portion 1311.

The auxiliary pulley fixing portion 1311 may be formed in a shape that protrudes from the pulley frame 1310 in a direction toward the connection portion 1400. For example, the auxiliary pulley fixing portion 1311 may be formed to protrude from one surface of the pulley frame 1310 in a direction toward the connection portion 1400. According to a preferred embodiment, the auxiliary pulley fixing portion 1311 may be disposed at the center of the pulley frame 1310.

In this case, the yaw pulley 1320 and the pitch pulley 1330 may be arranged on both sides of the auxiliary pulley fixing portion 1311, respectively. According to a preferred embodiment, the yaw pulley 1320 and the pitch pulley 1330 may be arranged at locations symmetrical around the auxiliary pulley fixing portion 1311. In other words, the first yaw wire 1361 and the second yaw wire 1362, and the first pitch wire 1363 and the second pitch wire 1364 entering the first auxiliary pulley 1351 may be distributed to both sides while passing through the auxiliary pulley fixing portion 1311. Therefore, it may be said that the first yaw wire 1361 and the second yaw wire 1362 extend toward the yaw pulley 1320, and the first pitch wire 1363 and the second pitch wire 1364 extend toward the pitch pulley 1330.

Therefore, the first yaw wire 1361 and the second yaw wire 1362 may approach to the yaw pulley 1320 at almost vertical angles, and the first pitch wire 1363 and the second pitch wire 1364 may approach to the pitch pulley 1330 at almost vertical angles. Preferably, the first yaw wire 1361 and the second yaw wire 1362 may approach to the yaw pulley 1320 vertically, and the first pitch wire 1363 and the second pitch wire 1364 may approach to the pitch pulley 1330 vertically. In other words, the first yaw wire 1361 and the second yaw wire 1362 may approach to the yaw pulley 1320 to form a tangent to the yaw pulley 1320, and the first pitch wire 1363 and the second pitch wire 1364 may approach to the pitch pulley 1330 to form a tangent to the pitch pulley 1330.

The first auxiliary pulley 1351 is disposed on the auxiliary pulley fixing portion 1311 and may change the paths of the first yaw wire 1361 and the second yaw wire 1362, and the first pitch wire 1363 and the second pitch wire 1364 extending to the power transmission portion 1300.

In detail, the first auxiliary pulley 1351 may be disposed between the yaw pulley 1320 and the pitch pulley 1330, as shown in FIG. 7.

A plurality of first auxiliary pulleys 1351 may be provided. The number of first auxiliary pulleys 1351 may at least correspond to the number of wires entering the power transmission portion 1300.

For example, the yaw wires may include a pair of the first yaw wire 1361 and the second yaw wire 1362 to enter the upper portion and the lower portion of the yaw pulley 1320, respectively. Also, the pitch wires may include a pair of the first pitch wire 1363 and the second pitch wire 1364 to enter the upper portion and the lower portion of the pitch pulley 1330, respectively. In other words, when four wires entering the power transmission portion 1300 are provided, four first auxiliary pulleys 1351 may be provided.

According to an optional embodiment, the plurality of first auxiliary pulleys 1351 may be arranged parallel to one another. The purpose of the arrangement is for the wires (e.g., the first yaw wire 1361, the second yaw wire 1362, the first pitch wire 1363, and the second pitch wire 1364) passing through the first auxiliary pulley 1351 to extend to the yaw pulley 1320 and/or pitch pulley 1330 in parallel or almost parallel with one another.

In detail, the wires (e.g., the first yaw wire 1361, the second yaw wire 1362, the first pitch wire 1363, and the second pitch wire 1364) extending from the end tool 1100 to the power transmission portion 1300 extend in parallel with one another. Therefore, since the plurality of first auxiliary pulleys 1351 are arranged in parallel with one another, the wires (e.g., the first yaw wire 1361, the second yaw wire 1362, the first pitch wire 1363, and the second pitch wire 1364) passing through the first auxiliary pulley 1351 may extend to the yaw pulley 1320 and/or the pitch pulley 1330 in parallel or almost parallel with one another.

According to an optional embodiment, the first auxiliary pulley 1351 may be disposed, such that a direction in which the wires (e.g., the first yaw wire 1361, the second yaw wire 1362, the first pitch wire 1363, and the second pitch wire 1364) are wound around the first auxiliary pulley 1351 and directions in which the wires (e.g., the first yaw wire 1361, the second yaw wire 1362, the first pitch wire 1363, and the second pitch wire 1364) are wound around the yaw pulley 1320 and the pitch pulley 1330 are almost perpendicular to each other, and preferably, are perpendicular to each other. In detail, with reference to FIG. 7, a direction in which the wires (e.g., the first yaw wire 1361, the second yaw wire 1362, the first pitch wire 1363, and the second pitch wire 1364) are wound around the first auxiliary pulley 1351 and directions in which the wires (e.g., the first yaw wire 1361, the second yaw wire 1362, the first pitch wire 1363, and the second pitch wire 1364) are wound around the yaw pulley 1320 and the pitch pulley 1330 may be perpendicular to each other. In other words, it may be said that the first auxiliary pulley 1351 changes directions in which the wires (e.g., the first yaw wire 1361, the second yaw wire 1362, the first pitch wire 1363, and the second pitch wire 1364) entering the first auxiliary pulley 1351 extend by 90°. In other words, it may be said that a groove formed on the first auxiliary pulley 1351 and grooves formed on the yaw pulley 1320 and the pitch pulley 1330 are perpendicular to each other.

By this configuration, paths of the wires (e.g., the first yaw wire 1361, the second yaw wire 1362, the first pitch wire 1363, and the second pitch wire 1364) entering the power transmission portion 1300 are changed through the first auxiliary pulley 1351, and the wires (e.g., the first yaw wire 1361, the second yaw wire 1362, the first pitch wire 1363, and the second pitch wire 1364) may approach toward the yaw pulley 1320 and the pitch pulley 1330 vertically or at almost vertical angles.

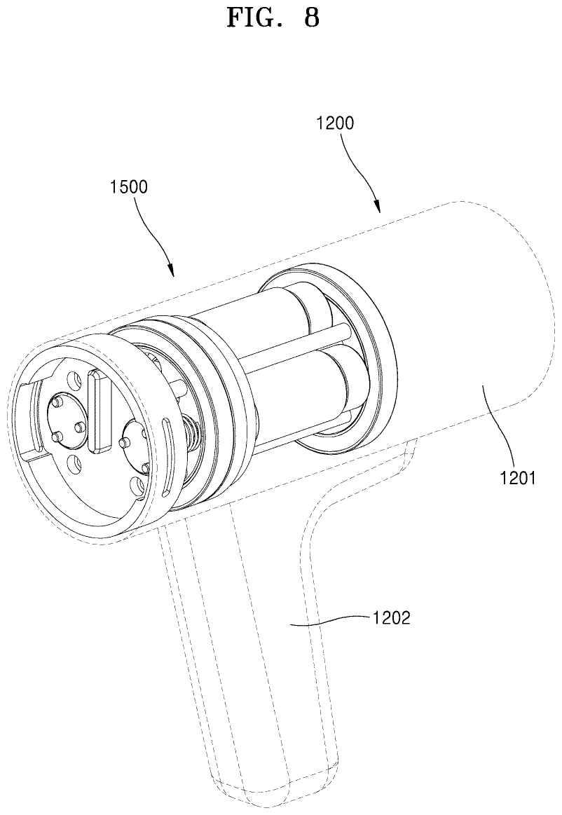

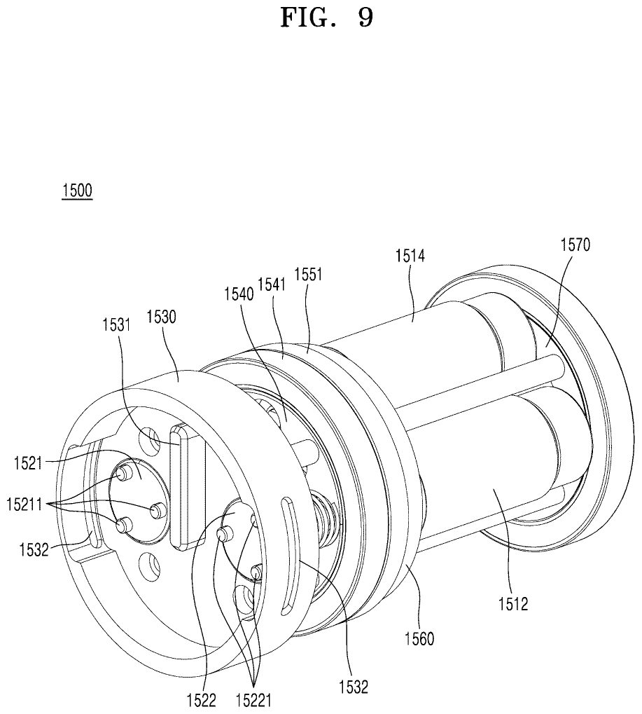

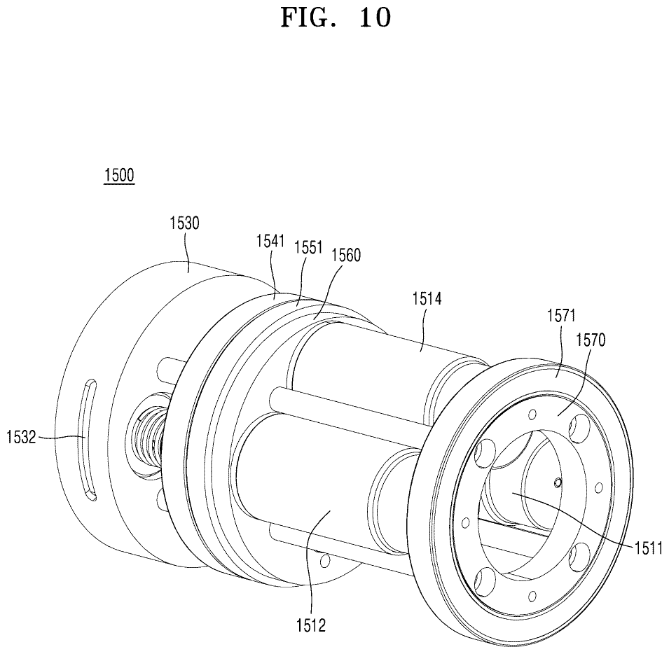

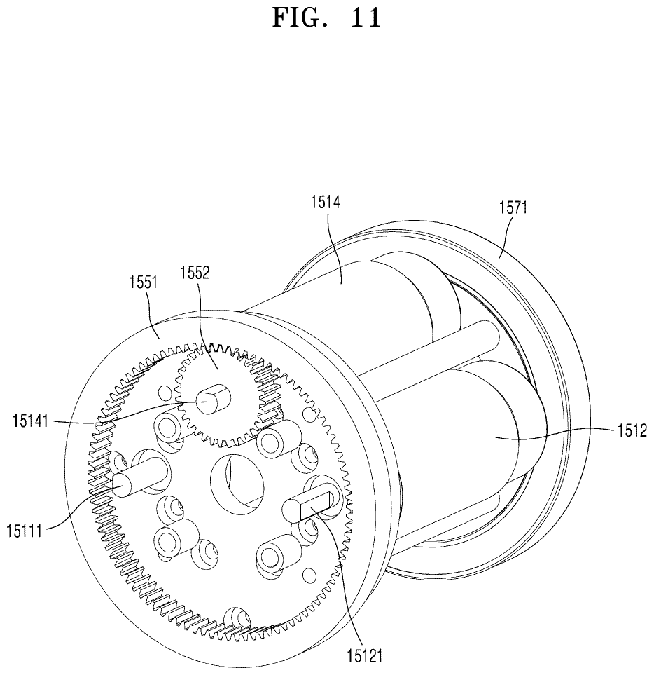

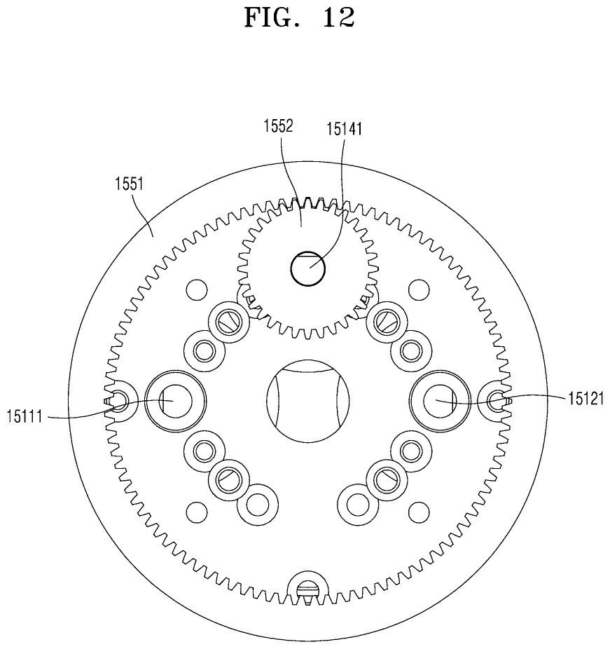

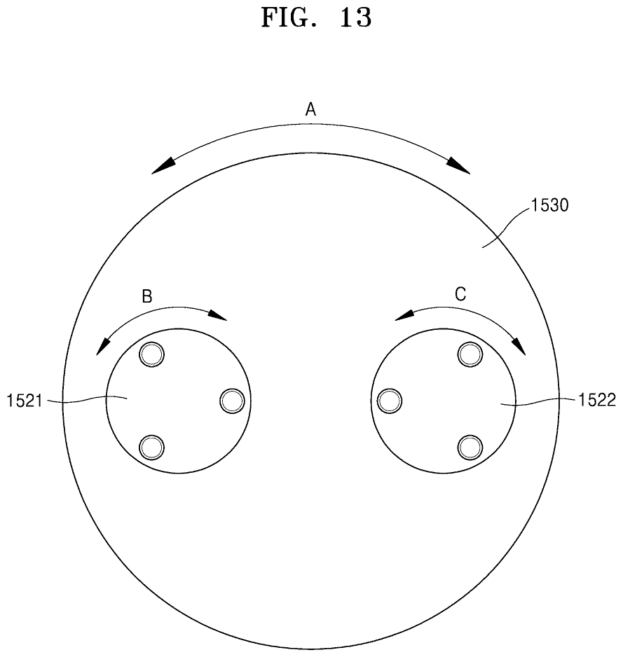

FIG. 8 is a diagram showing a manipulation portion 1200 and a power generation portion 1500 according to an embodiment of the present disclosure, FIG. 9 is a perspective view of the power generation portion 1500 of FIG. 8, and FIG. 10 is a rear view of the power generation portion 1500 of FIG. 9. FIG. 11 is a diagram for describing a gear structure of the power generation portion 1500 of FIG. 9, FIG. 12 is a front view of the structure of FIG. 11, and FIG. 13 is a diagram for describing the rotation of the power generation portion 1500 of FIG. 9.

Referring to FIGS. 8 to 13, the surgical instrument 1000 according to an embodiment of the present disclosure may include the power generation portion 1500 that generates power to control the end tool 1100.

The power generation portion 1500 may be disposed to be at least partially accommodated in the housing 1201 of the manipulation portion 1200. Here, the housing 1201 may refer to a component that forms the outer shape of the manipulation portion 1200, but, for a driving module for a surgical instrument, may refer to a component that forms the outer shape of a module body.

When a user manipulates the manipulation portion 1200, the power generation portion 1500 may generate power to control the end tool 1100 based on the manipulation of the user.

The power generation portion 1500 may include a motor pack 1510 including at least one motor.

The motor pack 1510 may roll around the axis in the direction in which the connection portion 1400 extends.

Here, the roll movement used in the present disclosure is defined as follows.

The roll movement refers to a movement that the end tool 1100, connection portion 1400, motor pack 1510, etc., which constitute the surgical instrument 1000, rotate around the axis in the direction in which the connection portion 1400 extends. In other words, the roll movement refers to a rotation without bending in the Y-axis direction of FIG. 3 or the Z-axis direction of FIG. 3 around the direction (X-axis direction of FIG. 3) in which the connection portion 1400 extends.

Referring back to FIG. 8, at least a portion of the power generation portion 1500 may be accommodated inside the housing 1201 of the manipulation portion 1200. In this case, the motor pack 1510 is accommodated inside the housing 1201 of the manipulation portion 1200. Here, the rolling of the motor pack 1510 may mean rotation of the motor pack 1510 along the inner circumferential surface of the housing 1201 inside the housing 1201. In other words, when the user holds the handle 1202 of the manipulation portion 1200 and performs a manipulation to roll the motor pack 1510, the motor pack 1510 may rotate around a direction in which the connection portion 1400 extends inside the housing 1201 while the housing 1201 and the handle 1202 of the manipulation portion 1200 are fixed in place. In other words, it may be said that, when the user performs a manipulation for roll movement of the motor pack 1510 while the user is holding the connection portion 1400 or fixing the connection portion 1400 to a particular position (e.g., fixing the connection portion 1400 to a separately provided robot or mount), the housing 1201 and the handle 1202 rotate.

The motor pack 1510 may include at least one motor. For example, the motor pack 1510 may include at least one motor that generates power to drive the end tool 1100 based on a signal input to the manipulation portion 1200.

According to an optional embodiment, the motor pack 1510 may further include a roll drive motor 1514.

Therefore, hereinafter, it should be understood that the motor pack 1510 includes at least one of the following motors: a yaw drive motor 1511, a pitch drive motor 1512, and the roll drive motor 1514, which will be described later.

The motor pack 1510 may include a yaw drive motor 1511. The yaw drive motor 1511 may generate power to yaw-rotate the end tool 1100. For example, the yaw drive motor 1511 may generate driving force for yaw-rotating the end tool 1100 when the user manipulates the manipulation portion 1200 to yaw-rotate the end tool 1100.

The driving force generated by the yaw drive motor 1511 may be transmitted to the power transmission portion 1300 and rotate the yaw pulley 1320, and, as the first yaw wire 1361 and the second yaw wire 1362 are moved by the rotation of the yaw pulley 1320, the end tool 1100 may yaw-rotate.

The yaw drive motor 1511 may include a yaw motor rotation shaft 15111 extending in one direction. The yaw motor rotation shaft 15111 is a part that rotates when the yaw drive motor 1511 starts driving. For example, the yaw motor rotation shaft 15111 may be formed to extend from the main body of the yaw drive motor 1511 in a direction toward the power transmission portion 1300. A yaw motor plate 1521 may be disposed at one end of the yaw motor rotation shaft 15111, as will be described later, and, when the yaw motor rotation shaft 15111 rotates, the yaw motor plate 1521 may rotate together. The yaw motor plate 1521 may be connected to the yaw pulley 1320, and driving force may be transmitted to the yaw pulley 1320 as the yaw motor plate 1521 rotates.

The motor pack 1510 may include a pitch drive motor 1512. The pitch drive motor 1512 may generate power to pitch-rotate the end tool 1100. For example, the pitch drive motor 1512 may generate driving force for pitch-rotating the end tool 1100 when the user manipulates the manipulation portion 1200 to pitch-rotate the end tool 1100.

The driving force generated by the pitch drive motor 1512 may be transmitted to the power transmission portion 1300 and rotate the pitch pulley 1330, and, as the pitch wires 1363 and 1364 are moved by the rotation of the pitch pulley 1330, the end tool 1100 may pitch-rotate.

The pitch drive motor 1512 may include a pitch motor rotation shaft 15121 extending in one direction. The pitch motor rotation shaft 15121 is a part that rotates when the pitch drive motor 1512 starts driving. For example, the pitch motor rotation shaft 15121 may be formed to extend from the main body of the pitch drive motor 1512 in a direction toward the power transmission portion 1300. A pitch motor plate 1522 may be disposed at one end of the pitch motor rotation shaft 15121, as will be described later, and, when the pitch motor rotation shaft 15121 rotates, the pitch motor plate 1522 may rotate together. The pitch motor plate 1522 may be connected to the pitch pulley 1330, and driving force may be transmitted to the pitch pulley 1330 as the pitch motor plate 1522 rotates.

The power generation portion 1500 may include the roll drive motor 1514 that generates power to roll-rotate the motor pack 1510. For example, the roll drive motor 1514 may generate driving force for roll-rotating the motor pack 1510 when a user manipulates the manipulation portion 1200 to rotate the motor pack 1510.

According to an embodiment, the roll drive motor 1514 may be provided in the motor pack 1510. For example, the roll drive motor 1514 may be provided inside the motor pack 1510 together with the yaw drive motor 1511 and the pitch drive motor 1512.

The roll drive motor 1514 may be driven by a user's manipulation to generate driving force to rotate the motor pack 1510. In this case, the roll drive motor 1514 may move together with the motor pack 1510. For example, when the motor pack 1510 is roll-rotated by the roll drive motor 1514, the roll drive motor 1514 may rotate together with the motor pack 1510 as a component included in the motor pack 1510.

According to another embodiment, the roll drive motor 1514 may not be provided in the motor pack 1510. For example, the roll drive motor 1514 may be disposed outside the motor pack 1510.

The roll drive motor 1514 may be driven by manipulation of a user to generate driving force to rotate the motor pack 1510. In this case, the roll drive motor 1514 may move independently from the motor pack 1510. For example, unlike as described later, the motor pack 1510 may be roll-rotated by the roll drive motor 1514, but the roll drive motor 1514 may not rotate together with the motor pack 1510. In detail, the yaw drive motor 1511 and the pitch drive motor 1512 may rotate together, but the roll drive motor 1514 may not rotate together with the yaw drive motor 1511 and the pitch drive motor 1512.

Hereinafter, for convenience of explanation, descriptions will be given under the assumption that the roll drive motor 1514 is provided in the motor pack 1510. However, one of ordinary skill in the art will understand that the roll drive motor 1514 may not be provided in the motor pack 1510 in the descriptions below. Also, one of ordinary skill in the art will understand that, when the roll drive motor 1514 is not provided in the motor pack 1510, descriptions given below may be appropriately modified and applied.

The motor pack 1510 may include a base plate 1560. The base plate 1560 may be disposed in front of the yaw drive motor 1511, the pitch drive motor 1512, and the roll drive motor 1514. The base plate 1560 may be connected to the roll drive motor 1514, the yaw drive motor 1511, and the pitch drive motor 1512. In other words, it may be said that the roll drive motor 1514, the yaw drive motor 1511, and the pitch drive motor 1512 are connected to the base plate 1560. In other words, it may be said that the base plate 1560 connects the roll drive motor 1514, the yaw drive motor 1511, and the pitch drive motor 1512 to one another, such that the roll drive motor 1514, the yaw drive motor 1511, and the pitch drive motor 1512 move or rotate together.

Therefore, when the base plate 1560 rotates due to the driving force of the roll drive motor 1514, the roll drive motor 1514, the yaw drive motor 1511, and the pitch drive motor 1512 connected to the base plate 1560 may simultaneously rotate. In other words, when the roll drive motor 1514 is driven, the base plate 1560 is rotated thereby, and, when the base plate 1560 rotates, the roll drive motor 1514, the yaw drive motor 1511, and the pitch drive motor 1512 connected to the base plate 1560 rotate together with the base plate 1560. Here, since the base plate 1560 rotates around the axis in the direction in which the connection portion 1400 extends, the motor pack 1510 including the base plate 1560, the roll drive motor 1514, the yaw drive motor 1511, and the pitch drive motor 1512 roll-rotates around the axis in the direction in which the connection portion 1400 extends.

According to an embodiment, the roll drive motor 1514, the yaw drive motor 1511, and the pitch drive motor 1512 may be arranged in parallel with one another. Also, the roll drive motor 1514, the yaw drive motor 1511, and the pitch drive motor 1512 may be arranged to form a circular pattern.

As described above, the motor pack 1510 may roll-rotate inside the housing 1201 of the manipulation portion 1200. In this case, since the motor pack 1510 includes a plurality of motors, the roll drive motor 1514, the yaw drive motor 1511, and the pitch drive motor 1512 may be arranged to form a circular pattern with one another, thereby minimizing the space needed by the motor pack 1510 to rotate. In other words, as the roll drive motor 1514, the yaw drive motor 1511, and the pitch drive motor 1512 are arranged to form a circular pattern, the housing 1201 may be designed to have a small inner diameter needed for rotation of the motor pack 1510, thereby contributing to the miniaturization and weight reduction of the surgical instrument 1000.

Meanwhile, here, arranging the roll drive motor 1514, the yaw drive motor 1511, and the pitch drive motor 1512 to form a circular pattern does not mean that they are arranged at equal intervals between one another, and any arrangement in which the outer circumferential surfaces of the roll drive motor 1514, the yaw drive motor 1511, and the pitch drive motor 1512 are arranged within one circle will suffice.

According to an embodiment, the roll drive motor 1514, the yaw drive motor 1511, and the pitch drive motor 1512 may be provided to have different performances. For example, amounts of driving force that need to be generated by the roll drive motor 1514, the yaw drive motor 1511, and the pitch drive motor 1512 to perform their respective roles may be different from one another. To this end, the roll drive motor 1514, the yaw drive motor 1511, and the pitch drive motor 1512 may have different outputs or different sizes as needed.

At least one through hole may be formed in the base plate 1560. For example, at least as many through holes as the number of motors included in the motor pack 1510 may be formed in the base plate 1560. The through hole is a hole through which the rotation shaft of each motor passes.

For example, the yaw motor rotation shaft 15111 extends from the main body of the yaw drive motor 1511 and may be formed to extend through the base plate 1560 (through a through hole). Also, the pitch motor rotation shaft 15121 extends from the main body of the pitch drive motor 1512 and may be formed to extend through the base plate 1560 (through a through hole).

Meanwhile, the roll drive motor 1514 may include a roll motor rotation shaft 15141 extending in one direction. The roll motor rotation shaft 15141 is a part that rotates when the roll drive motor 1514 starts driving. For example, the roll motor rotation shaft 15141 may be formed to extend in a forward direction from the main body of the roll drive motor 1514. In other words, the roll motor rotation shaft 15141 extends forward from the main body of the roll drive motor 1514 and may be formed to extend through the base plate 1560.

Hereinafter, the principle of rotation of the motor pack 1510 will be described in detail.

Referring to FIGS. 11 and 12, the power generation portion 1500 may include a first gear 1551 formed in a circular shape and a second gear 1552 engaged with the first gear 1551.

The first gear 1551 is formed in the shape of a hollow circle, and gear teeth may be formed on the inner circumferential surface of the hollow circle. In other words, the first gear 1551 may be a ring gear with gear teeth formed on the inner circumferential surface thereof.

The second gear 1552 is a gear with gear teeth formed on the outer circumferential surface thereof and may be engaged with the first gear 1551. The second gear 1552 may be disposed on the roll motor rotation shaft 15141. In other words, the roll motor rotation shaft 15141 is formed to extend forward from the main body of the roll drive motor 1514 to penetrate through the base plate 1560, and the second gear 1552 may be disposed at an extended portion of the roll motor rotation shaft 15141. At this time, the second gear 1552 is coupled to the roll motor rotation shaft 15141 and, when the roll motor rotation shaft 15141 rotates, the second gear 1552 may rotate together.

The first gear 1551 may be disposed in front of the base plate 1560. Also, the first gear 1551 may be fixed to the inner circumferential surface of the housing 1201. Therefore, when the roll drive motor 1514 is driven, the motor pack 1510 may roll-rotate within the housing 1201.

In detail, when the roll drive motor 1514 is driven, the roll motor rotation shaft 15141 may rotate, and the second gear 1552 disposed on the roll motor rotation shaft 15141 may rotate together. At this time, when the second gear 1552 rotates, the first gear 1551 engaged with the second gear 1552 is fixed to the inner circumferential surface of the housing 1201, and thus the second gear 1552 moves along the gear teeth of the first gear 1551. In other words, when the roll drive motor 1514 starts driving, the first gear 1551 and the second gear 1552 rotate relatively. At this time, since the first gear 1551 is fixed to the housing 1201, the second gear 1552 relatively moves along the first gear 1551. Also, the second gear 1552 is connected to the roll motor rotation shaft 15141, the roll drive motor 1514 is connected to the base plate 1560, and the base plate 1560 is connected to the yaw drive motor 1511 and the pitch drive motor 1512. Therefore, the motor pack 1510 may rotate relative to the housing 1201 due to the movements of the first gear 1551 and the second gear 1552. In other words, the motor pack 1510 may rotate independently of the movement of the housing 1201.

To explain this in more detail, the base plate 1560 may rotate relative to the housing 1201. In other words, since the second gear 1552 is connected to the base plate 1560 by the roll motor rotation shaft 15141, when the second gear 1552 moves, as the second gear 1552 moves along the first gear 1551, the base plate 1560 rotates relative to the housing 1201. Here, since the roll motor rotation shaft 15141 is eccentric with respect to the rotation shaft of the base plate 1560, when the second gear 1552 moves along the first gear 1551, the base plate 1560 may rotate relative to the housing 1201 instead of changing its position to follow the second gear 1552.

Meanwhile, as will be described later, a bearing plate 1540 may also be connected to the second gear 1552 through the roll motor rotation shaft 15141. Therefore, when the second gear 1552 rotates, the bearing plate 1540 may rotate relative to the housing 1201 as the second gear 1552 moves along the first gear 1551. In this case, as will be described later, a bearing 1541 to reduce rotational friction of the bearing plate 1540 may be disposed coaxially with the bearing plate 1540 and in contact with the inner circumferential surface of the housing 1201. Therefore, the bearing plate 1540 may easily rotate.

Meanwhile, although gear teeth of the first gear 1551 and the second gear 1552 are shown in the shape of spur gears in the drawings, the present disclosure is not limited thereto, and the first gear 1551 and the second gear 1552 may have various shapes such as helical gears and herringbone gears.

Meanwhile, when the roll drive motor 1514 is disposed outside the motor pack 1510, the first gear 1551 and the second gear 1552 may be formed at different positions.

According to the other embodiment, the power generation portion 1500 may further include the bearing plate 1540 and a first bearing 1541. The bearing plate 1540 and the first bearing 1541 may reduce rotational friction between the motor pack 1510 and the housing 1201 when the motor pack 1510 roll-rotates.

The bearing plate 1540 may be disposed in front of the first gear 1551.

At least one through hole may be formed in the bearing plate 1540. The through hole is a hole through which the rotation shafts of respective motors pass.

For example, the yaw motor rotation shaft 15111 may be formed to extend from the main body of the yaw drive motor 1511 to penetrate through the base plate 1560 and the bearing plate 1540. Also, the pitch motor rotation shaft 15121 may be formed to extend from the main body of the pitch drive motor 1512 to penetrate through the base plate 1560 and the bearing plate 1540. In this case, the roll motor rotation shaft 15141 extends from the main body of the roll drive motor 1514 to penetrate through the base plate 1560, but may not extend to the bearing plate 1540.

In this way, since the yaw motor rotation shaft 15111 and the pitch motor rotation shaft 15121 are formed to extend through the bearing plate 1540, when the motor pack 1510 rotates, the base plate 1560 and the bearing plate 1540 may rotate together.

The first bearing 1541 may be disposed on an outer circumferential surface of the bearing plate 1540. For example, the first bearing 1541 may be disposed to cover the outer circumferential surface of the bearing plate 1540. Therefore, when the motor pack 1510 roll-rotates, the bearing plate 1540 rotates together with the motor pack 1510, and, at this time, the bearing plate 1540 and the first bearing 1541 may reduce rotational friction between the motor pack 1510 and the housing 1201.

According to the other embodiment, the power generation portion 1500 may further include a circuit plate 1570 and a second bearing 1571. The circuit plate 1570 and the second bearing 1571 may reduce rotational friction between the motor pack 1510 and the housing 1201 when the motor pack 1510 roll-rotates.

The circuit plate 1570 may be disposed behind the motor pack 1510.

The circuit plate 1570 is a part to which circuit units are connected, as will be described later.

The circuit plate 1570 is connected to the motor pack 1510 and may rotate together when the motor pack 1510 rotates.

The second bearing 1571 may be disposed on the outer circumferential surface of the circuit plate 1570. Therefore, when the motor pack 1510 roll-rotates, the circuit plate 1570 rotates together with the motor pack 1510, and, at this time, the circuit plate 1570 and the second bearing 1571 may reduce rotational friction between the motor pack 1510 and the housing 1201.

The power generation portion 1500 may further include a pulley coupling plate 1530.

The pulley coupling plate 1530 is a part to which the power transmission portion 1300 is connected.

According to an embodiment, the power transmission portion 1300 may be detachably coupled to the power generation portion 1500. For example, the power transmission portion 1300 may be detachably coupled to the pulley coupling plate 1530. Therefore, after a user uses components (the power transmission portion 1300, the connection portion 1400, and the end tool 1100) in the direction from the power transmission portion 1300 toward the distal end, the user may discard the components, couple a new product to the manipulation portion 1200 accommodating the power generation portion 1500, and use the surgical instrument 1000 again.

According to an embodiment, at least one coupling member 1316 may be formed at the pulley frame 1310 of the power transmission portion 1300. A hook 13161 may be formed at the at least one coupling member 1316. Also, the pulley coupling plate 1530 may include an internal space for accommodating at least a portion of the pulley frame 1310 and a wall formed along the circumference of the pulley coupling plate 1530 to define the internal space. At this time, a hook groove 1532 in which the hook 13161 is inserted and fixed may be formed on the wall. Therefore, when the pulley frame 1310 is inserted into the inner space of the pulley coupling plate 1530, the hook 13161 may be inserted into the hook groove 1532 and fixed.

According to an embodiment, the pulley coupling plate 1530 may include a protruding coupling block 1531, and the pulley frame 1310 may include an insertion groove into which the coupling block 1531 is inserted. Therefore, when the pulley coupling plate 1530 and the pulley frame 1310 are coupled, the coupling block 1531 is inserted into the insertion groove, and thus the pulley coupling plate 1530 and the pulley frame 1310 may be coupled to each other at a predetermined position.

Also, since the coupling block 1531 may be inserted into the insertion groove, the roll rotational force of the pulley coupling plate 1530 may be transmitted to the pulley frame 1310 through the coupling block 1531 and the insertion groove. According to an optional embodiment, the coupling block 1531 may be formed in the shape of a long bar, and, in this case, the insertion groove may be formed in a shape corresponding to the shape of the coupling block 1531. According to the other embodiment, although not shown in the drawings, the surgical instrument 1000 according to the present disclosure may further include a waterproof structure.

According to a specific example, at least one O-ring may be provided inside the housing 1201.

For example, an O-ring may be provided between the outer circumferential surface of the pulley coupling plate 1530 and the housing 1201. The O-ring is disposed on the outer circumferential surface of the pulley coupling plate 1530 to be in close contact with the housing 1201 to prevent water, etc. from penetrating between the power generation portion 1500 and the housing 1201. In another example, an O-ring may be provided at least at one location between the yaw motor plate 1521 and the pulley coupling plate 1530 and between the pitch motor plate 1522 and the pulley coupling plate 1530.

An O-ring may be disposed to fit tightly between the yaw motor plate 1521 or the pitch motor plate 1522 and the pulley coupling plate 1530 to prevent water, etc. from penetrating between the yaw motor plate 1521, the pitch motor plate 1522 and the pulley coupling plate 1530. According to an embodiment, the pulley coupling plate 1530 and the bearing plate 1540 may be coupled to each other by at least one bolt.

In this case, at least one seal washer may be disposed under the bolt in a bolt hole into which the bolt is inserted. The seal washer may prevent water, etc. from penetrating through the bolt hole. At least one through hole may be formed in the pulley coupling plate 1530.

For example, two through holes may be formed in the pulley coupling plate 1530. The yaw motor plate 1521 and the pitch motor plate 1522 may be arranged in the through holes formed in the pulley coupling plate 1530.

The yaw motor plate 1521 may be rotated by driving force generated by the yaw drive motor 1511.

The yaw motor plate 1521 may be disposed at one end of the yaw motor rotation shaft 15111. For example, when the yaw motor rotation shaft 15111 rotates, the yaw motor plate 1521 may rotate together. In other words, it may be said that the yaw motor plate 1521 is a member that transmits driving force generated by the yaw drive motor 1511 to the power transmission portion 1300. At least one first protrusion 15211 may be formed on the yaw motor plate 1521.

The at least one first protrusion 15211 is a portion that protrudes outward from the yaw motor plate 1521. The at least one first protrusion 15211 may be inserted into at least one first insertion hole 13131 formed in a yaw pulley plate 1313, as described later. The pitch motor plate 1522 may be rotated by driving force generated by the pitch drive motor 1512.

The pitch motor plate 1522 may be disposed at one end of the pitch motor rotation shaft 15121. For example, when the pitch motor rotation shaft 15121 rotates, the pitch motor plate 1522 may rotate together. In other words, it may be said that the pitch motor plate 1522 is a member that transmits driving force generated by the pitch drive motor 1512 to the power transmission portion 1300. At least one second protrusion 15221 may be formed on the pitch motor plate 1522.

The at least one second protrusion 15221 is a portion that protrudes outward from the pitch motor plate 1522. The at least one second protrusion 15221 may be inserted into at least one second insertion hole 13141 formed in a pitch pulley plate 1314, as described later. The yaw pulley plate 1313 and the pitch pulley plate 1314 may be arranged at the pulley frame 1310. The yaw pulley plate 1313 may be formed to be rotatable.

In detail, the yaw pulley plate 1313 may be coupled to the yaw motor plate 1521 and may rotate together when the yaw motor plate 1521 rotates. The yaw pulley plate 1313 is a part connected to the yaw pulley 1320, and, when the yaw pulley plate 1313 rotates, the yaw pulley 1320 may rotate together. In other words, when power transmitted from the outside rotates the yaw pulley plate 1313, the yaw pulley 1320 may rotate together. In other words, it may be said that the yaw pulley plate 1313 is a part that receives driving force generated by the yaw drive motor 1511 and transmits the driving force to the yaw pulley 1320. The yaw pulley plate 1313 may include at least one first insertion hole 13131.

The at least one first insertion hole 13131 is a part into which the at least one first protrusion 15211 of the yaw motor plate 1521 is inserted. In this way, the yaw motor plate 1521 and the yaw pulley plate 1313 may be stably coupled to each other through coupling between the at least one first protrusion 15211 and the at least one first insertion hole 13131, and driving force of the yaw drive motor 1511 may be efficiently transmitted to the yaw pulley 1320. The pitch pulley plate 1314 may be formed to be rotatable.

In detail, the pitch pulley plate 1314 may be coupled to the pitch motor plate 1522 and may rotate together when the pitch motor plate 1522 rotates. The pitch pulley plate 1314 is a part connected to the pitch pulley 1330, and, when the pitch pulley plate 1314 rotates, the pitch pulley 1330 may rotate together. In other words, when power transmitted from the outside rotates the pitch pulley plate 1314, the pitch pulley 1330 may rotate together. In other words, it may be said that the pitch pulley plate 1314 is a part that receives driving force generated by the pitch drive motor 1512 and transmits the driving force to the pitch pulley 1330. The pitch pulley plate 1314 may include at least one second insertion hole 13141.

The at least one second insertion hole 13141 is a part into which the at least one second protrusion 15221 of the pitch motor plate 1522 is inserted. In this way, the pitch motor plate 1522 and the pitch pulley plate 1314 may be stably coupled to each other through coupling between the at least one second protrusion 15221 and the at least one second insertion hole 13141, and driving force of the pitch drive motor 1512 may be efficiently transmitted to the pitch pulley 1330. According to the other embodiment, the yaw drive motor 1511, the pitch drive motor 1512, and the roll drive motor 1514 may be driven independently of one another.

Therefore, the yaw drive motor 1511, the pitch drive motor 1512, and the roll drive motor 1514 may perform the yaw rotation of the end tool 1100, the pitch rotation of the end tool 1100, and the roll rotation of motor pack 1510 independently of one another. Referring back to FIG. 13, the pulley coupling plate 1530 may rotate in a first direction A as the roll drive motor 1514 is driven.

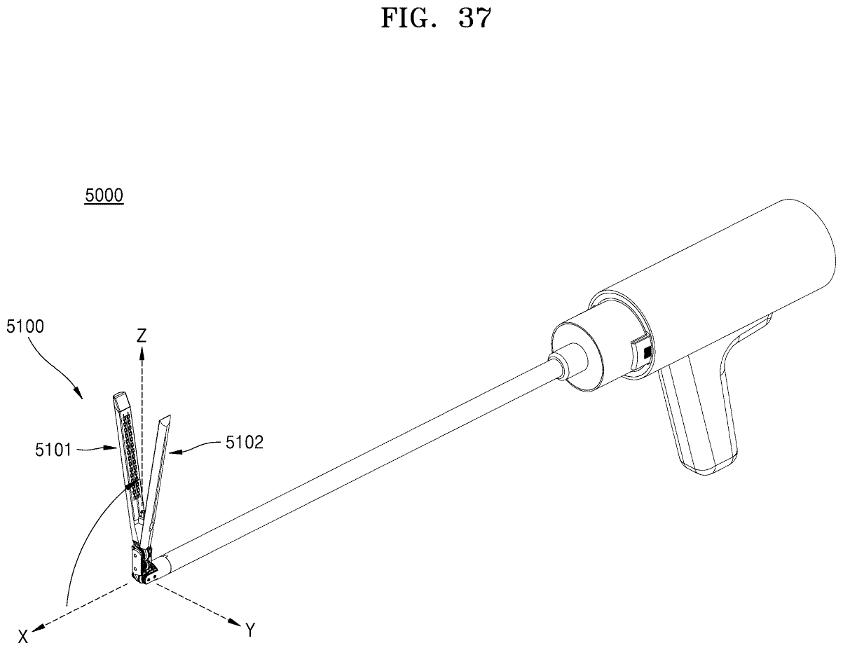

At this time, since the yaw drive motor 1511 may be driven independently, the yaw drive motor 1511 may be driven independently regardless of the driving of the roll drive motor 1514 and rotate the yaw motor plate 1521 in a second direction B. Also, since the pitch drive motor 1512 may be driven independently, the pitch drive motor 1512 may be driven independently regardless of the driving of the roll drive motor 1514 and the yaw drive motor 1511 and rotate the pitch motor plate 1522 in a third direction C. In other words, the end tool 1100 may perform only one of pitch rotation, yaw rotation, and roll rotation or may perform multiple rotations simultaneously. FIGS. 37 to 41 are diagrams showing the pitch rotation motion of a surgical instrument according to the present disclosure.

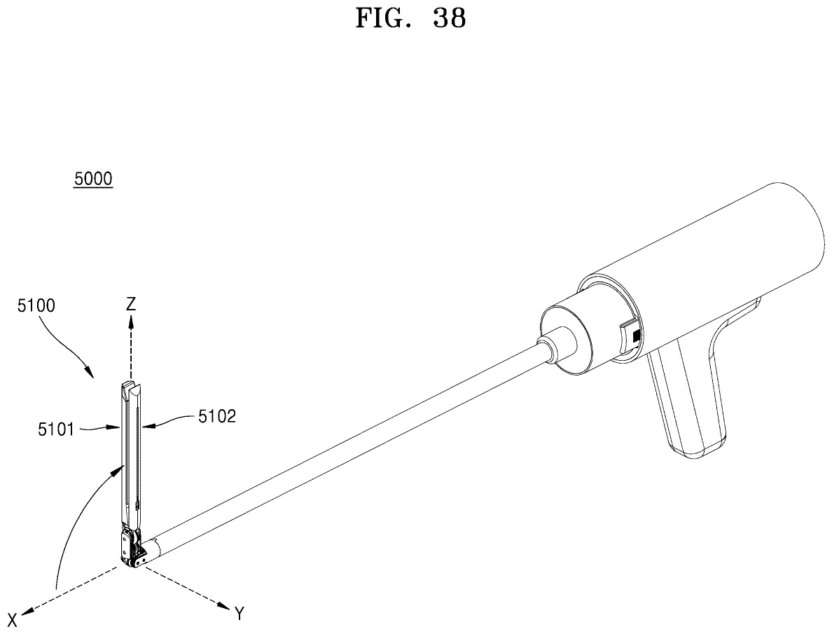

In detail, FIG. 37 is a diagram showing a state in which jaws are pitch-rotated by −90°, and FIG. 38 is a diagram showing a process of performing an actuation motion in the state in which jaws are pitch-rotated by −90°.

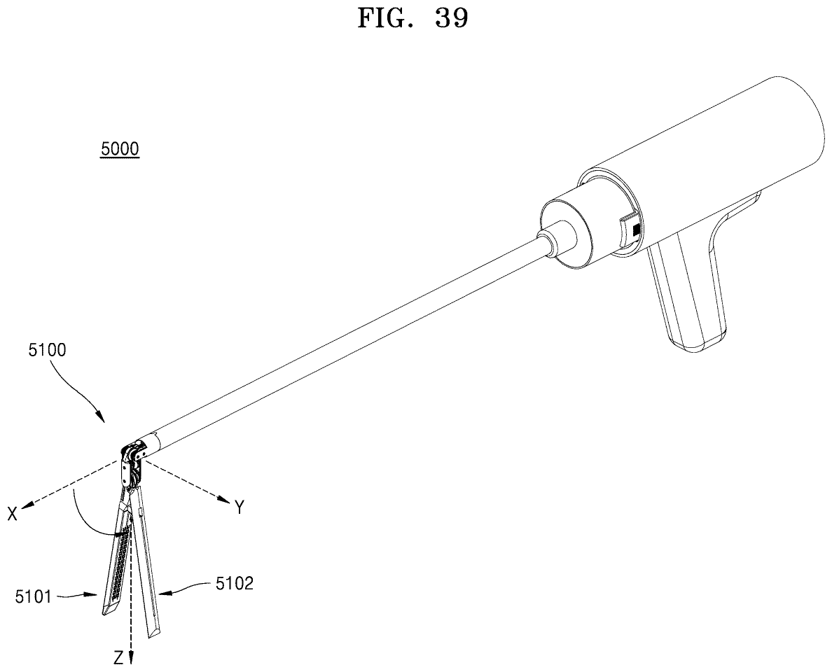

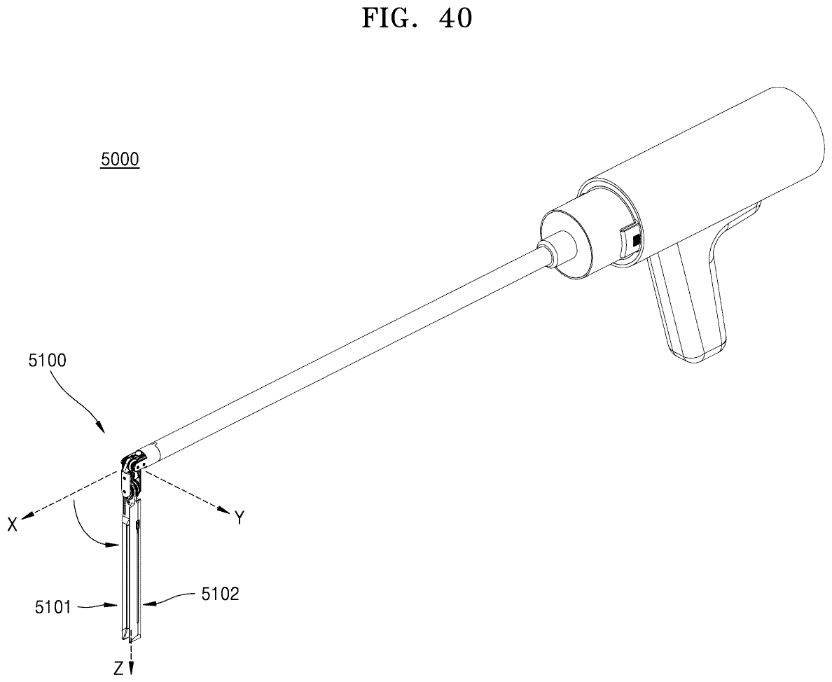

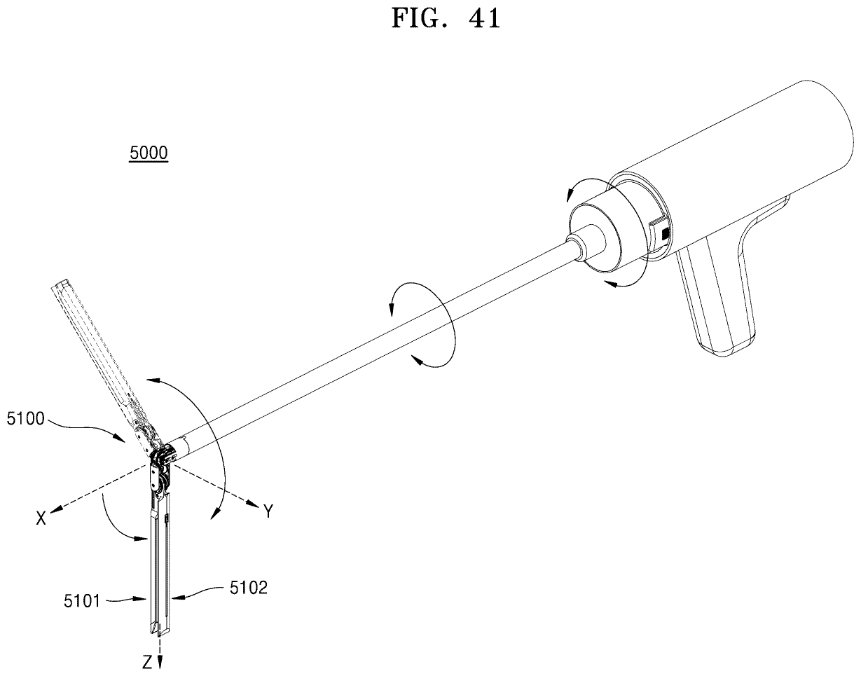

FIG. 39 is a diagram showing a state in which jaws are pitch-rotated by +90°, FIG. 40 is a diagram showing a process of performing an actuation motion in the state in which jaws are pitch-rotated by +90°, and FIG. 41 is a diagram showing a state in which a roll motion is performed while jaws are pitch-rotated. Referring to FIGS. 37 to 41, a surgical instrument 5000 according to the present disclosure may include an end tool 5100 including a first jaw 5101 and a second jaw 5102.

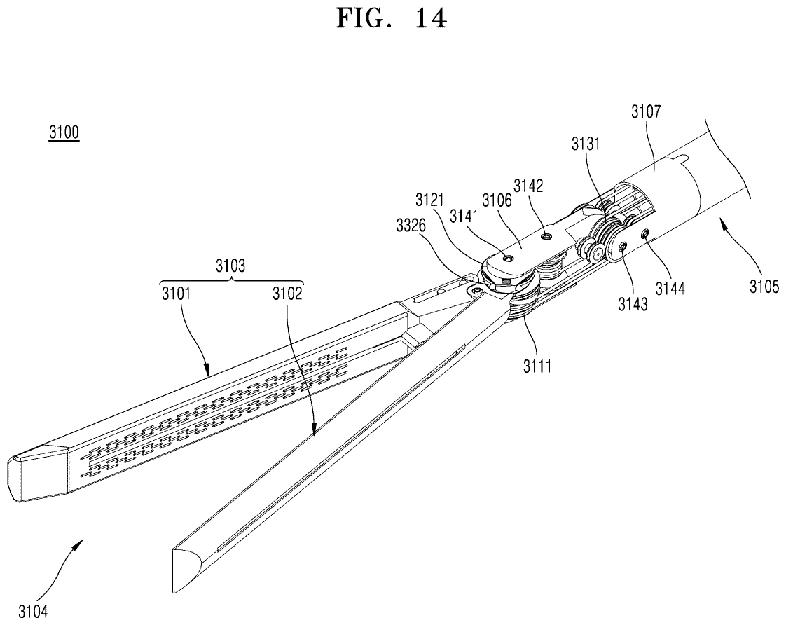

Here, the end tool 5100 of the surgical instrument 5000 may correspond to the end tool 1100 described above with reference to FIGS. 1 to 4. Alternatively, the end tool 5100 of the surgical instrument 5000 may correspond to the end tool 3100 described above with reference to FIGS. 14 to 18. Alternatively, the end tool 5100 of the surgical instrument 5000 may correspond to the end tool 1100 or the end tool 3100, from which at least some components are modified or omitted. The end tool 5100 of the surgical instrument 5000 may pitch-rotate in the +direction around the pitch rotation shaft (Y-axis).

At this time, while the end tool 5100 is pitch-rotated in the +direction around the pitch rotation shaft (Y-axis), the first jaw 5101 and the second jaw 5102 of the end tool 5100 may perform an actuation motion. Also, the end tool 5100 of the surgical instrument 5000 may pitch-rotate in the −direction around the pitch rotation shaft (Y-axis).

At this time, while the end tool 5100 is pitch-rotated in the −direction around the pitch rotation shaft (Y-axis), the first jaw 5101 and the second jaw 5102 of the end tool 5100 may perform an actuation motion. Here, the rotation angle of the end tool 5100 may be set variously depending on the ratio of pulleys.

On the other hand, the end tool 5100 of the surgical instrument 5000 may roll-rotate around the roll rotation shaft (X-axis) when the end tool 5100 did not rotate around the pitch rotation shaft (Y-axis), after pitch-rotating in the +direction, or after pitch-rotating in the −direction.

At this time, the end tool 5100 may roll-rotate with the first jaw 5101 and the second jaw 5102 spread apart from each other or may roll-rotate while the first jaw 5101 and the second jaw 5102 are performing an actuation motion. Here, the end tool 5100 is mechanically connected to a motor pack of a power generation portion and may rotate together with the motor pack.

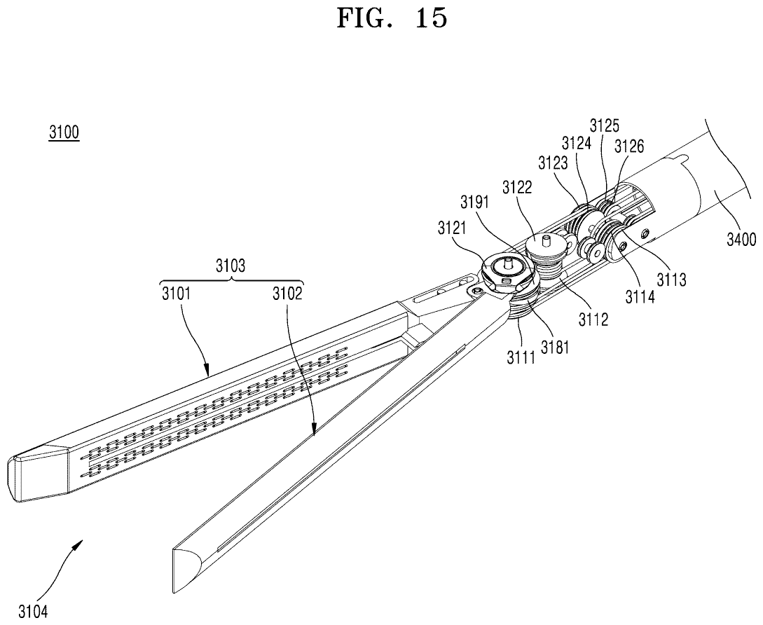

When the motor pack of the power generation portion rolls, the power transmission portion connected to the power generation portion, the connection portion connected to the power transmission portion, and the end tool 5100 formed on one side of the connection portion may rotate simultaneously. FIG. 14 is a perspective view of an end tool 3100 according to another embodiment of the present disclosure, and FIG. 15 is a perspective view of the end tool 3100 of FIG. 14 from which an end tool hub 3106 and a pitch hub 3107 are removed.

Referring to FIGS. 14 and 15, an end tool 3100 of a surgical instrument according to another embodiment of the present disclosure may include a first jaw 3101 and a second jaw 3102 formed to be rotatable.

In other words, the end tool 3100 of the surgical instrument according to another embodiment of the present disclosure may include a pair of jaws for performing a grip operation, that is, a first jaw 3101 and a second jaw 3102.

Here, the first jaw 3101, the second jaw 3102, or components including the first jaw 3101 and the second jaw 3102 may be referred to as two jaw 3103. Here, the end tool 3100 of a surgical instrument 3000 according to an embodiment of the present disclosure is formed to be rotatable in at least one direction. For example, the end tool 3100 may be formed to perform a pitch motion around the Y-axis (refer to FIG. 3) and, at the same time, perform a yaw motion and an actuation motion around the Z-axis of (refer to FIG. 3).

The end tool 3100 may include a plurality of pulleys including a first pulley 3111 related to the rotational movement of the first jaw 3101.

Also, the end tool 3100 may include a plurality of pulleys including a second pulley 3121 related to the rotational movement of the second jaw 3102. Here, the drawings show that the pulleys facing each other are formed in parallel to each other. However, the present disclosure is not limited thereto, and pulleys may be formed at various positions and with various sizes suitable for the configuration of the end tool.

Also, the end tool 3100 according to the present embodiment may include an end tool hub 3106 and a pitch hub 3107.

A first rotation shaft 3141 and a second rotation shaft 3142 are inserted through the end tool hub 3106, and the end tool hub 3106 may also accommodate at least portions of one or more pulleys axially coupled to the first rotation shaft 3141 therein.

Also, the end tool hub 3106 may accommodate at least portions of one or more pulleys axially coupled to the second rotation shaft 3142 therein. Meanwhile, a third pulley 3131 serving as an end tool pitch pulley may be formed at one end of the end tool hub 3106.

The third pulley 3131 may be formed as one body with the end tool hub 3106. In other words, a pulley having a disk-like shape may be formed at one end of the end tool hub 3106, and a groove in which a wire may be wound may be formed on the outer circumferential surface of the pulley. Alternatively, the third pulley 3131 may be formed as a member separate from the end tool hub 3106 and coupled to the end tool hub 3106. A third rotation shaft 3143 and a fourth rotation shaft 3144 are inserted through the pitch hub 3107, and the pitch hub 3107 may be axially coupled to the end tool hub 3106 (and the third pulley 3131) by the third rotation shaft 3143.

Therefore, the end tool hub 3106 and the third pulley 3131 may be formed to be rotatable with respect to the pitch hub 3107 around the third rotation shaft 3143. Also, the pitch hub 3107 may accommodate at least portions of one or more pulleys axially coupled to the third rotation shaft 3143 therein.

Also, the pitch hub 3107 may accommodate at least portions of one or more pulleys axially coupled to the fourth rotation shaft 3144 therein. Also, the end tool 3100 according to present embodiment may include the first rotation shaft 3141, the second rotation shaft 3142, the third rotation shaft 3143, and the fourth rotation shaft 3144.

As described above, the first rotation shaft 3141 and the second rotation shaft 3142 may be inserted through the end tool hub 3106, and the third rotation shaft 3143 and the fourth rotation shaft 3144 may be inserted through the pitch hub 3107. The first rotation shaft 3141, the second rotation shaft 3142, the third rotation shaft 3143, and the fourth rotation shaft 3144 may be sequentially arranged in a direction from a distal end 3104 of the end tool 3100 to the proximal end 3105 of the end tool 3100.

Therefore, sequentially from the distal end 3104, the first rotation shaft 3141 may be referred to as a first pin, the second rotation shaft 3142 may be referred to as a second pin, the third rotation shaft 3143 may be referred to as a third pin, and the fourth rotation shaft 3144 may be referred to as a fourth pin. Here, the first rotation shaft 3141 may function as an end tool jaw pulley rotation shaft, the second rotation shaft 3142 may function as an end tool jaw auxiliary pulley rotation shaft, the third rotation shaft 3143 may function as an end tool pitch rotation shaft, and the fourth rotation shaft 3144 may function as an end tool pitch auxiliary rotation shaft of the end tool 3100.

One or more pulleys may be fitted to each of the first rotation shaft 3141, the second rotation shaft 3142, the third rotation shaft 3143, and the fourth rotation shaft 3144.

The first pulley 3111 may function as an end tool first jaw pulley, and the second pulley 3121 may function as an end tool second jaw pulley.

The first pulley 3111 may be referred to as a first jaw pulley, the second pulley 3121 may be referred to as a second jaw pulley, and the two components may be collectively referred to as end tool jaw pulleys or simply jaw pulleys. The first pulley 3111 and the second pulley 3121, which are end tool jaw pulleys, are formed to face each other and are formed to be rotatable independently of each other around the first rotation shaft 3141, which is an end tool jaw pulley rotation shaft.

At this time, the first pulley 3111 and the second pulley 3121 are formed to be a certain distance apart from each other, and a staple assembly accommodating portion may be formed therebetween. Also, at least portions of a staple pulley assembly and a staple link assembly for a staple motion, which will be described later, may be arranged in the staple assembly accommodating portion. Here, although the drawings show that the first pulley 3111 and the second pulley 3121 are formed to rotate around a single rotation shaft (e.g., the first rotation shaft 3141), end tool jaw pulleys may also be formed to be rotatable around separate rotation shafts.

Here, the first jaw 3101 may be fixedly coupled to the first pulley 3111 and rotate together with the first pulley 3111, and the second jaw 3102 may be fixedly coupled to the second pulley 3121 and rotate together with the second pulley 3121. A yaw motion and an actuation motion of the end tool 3100 are performed according to rotations of the first pulley 3111 and the second pulley 3121. In other words, a yaw motion is performed when the first pulley 3111 and the second pulley 3121 rotate in the same direction around the first rotation shaft 3141, and an actuation motion is performed when the first pulley 3111 and the second pulley 3121 rotate in directions opposite to each other around the first rotation shaft 3141. Here, the first jaw 3101 and the first pulley 3111 may be formed as separate members and coupled to each other, or the first jaw 3101 and the first pulley 3111 may be formed as one body.

In the same regard, the second jaw 3102 and the second pulley 3121 may be formed as separate members and coupled to each other, or the second jaw 3102 and the second pulley 3121 may be formed as one body. Also, one or more auxiliary pulleys may be arranged adjacent to the first pulley 3111 and the second pulley 3121.

These pulleys are formed, such that one or more wires are wound around each of the pulleys, the pulleys may be rotated by wires, and the wires may move along the pulleys. Therefore, power may be transmitted to the end tool 3100.

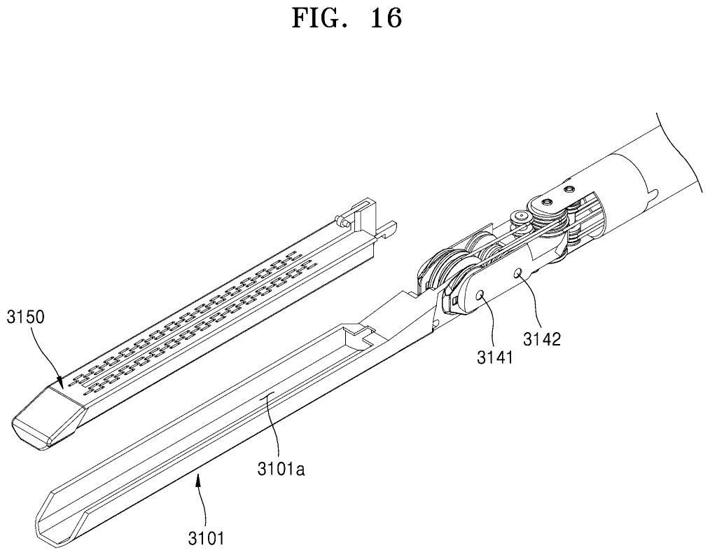

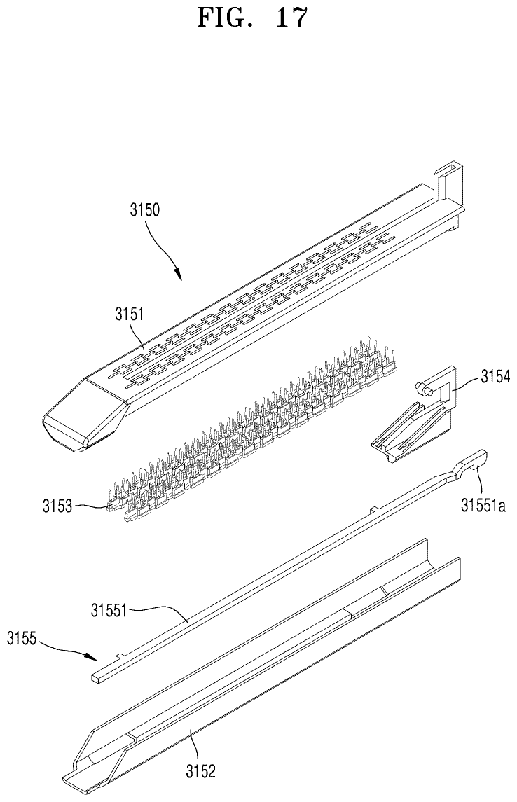

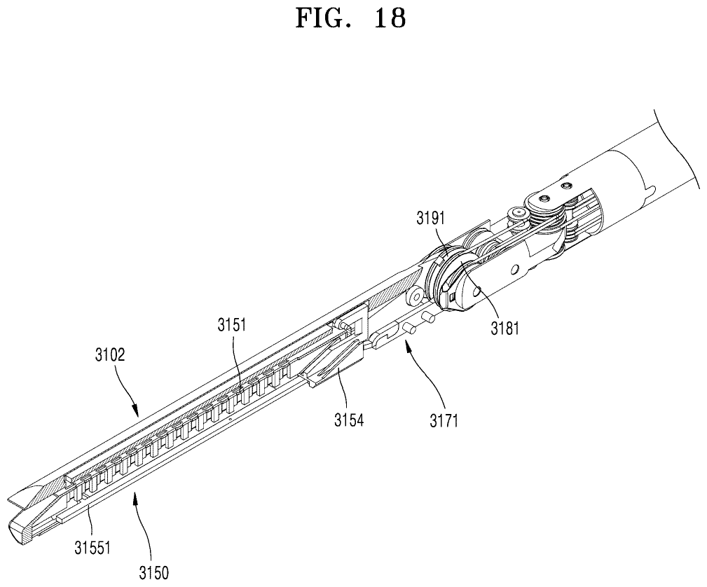

FIG. 16 is a perspective view of a first jaw 3101 and a cartridge 3150 of the end tool 3100 of FIG. 14, FIG. 17 is an exploded perspective view of the cartridge 3150 of FIG. 16, and FIG. 18 is a perspective cross-sectional view for describing the internal structure of the cartridge 3150 of FIG. 16.

Referring to FIGS. 16 to 18, a cartridge 3150 may be formed to be attachable to and detachable from the first jaw 3101, and includes a plurality of staples 3153 and a blade (not shown) therein to suture and cut tissues.

Here, the cartridge 3150 may include a cover 3151, a housing 3152, the plurality of staples 3153, a drawing member (not shown), a working member 3154, a moving member 3155, etc. The housing 3152 may form the outer shape of the cartridge 3150 and may be formed to have an overall hollow box-like shape from which one surface (top surface) is removed to accommodate the moving member 3155, the working member 3154, and the plurality of staples 3153 therein.

Here, the housing 3152 may have a cross-section having an approximately a ‘U’-like shape. The cover 3151 is formed to cover the upper portion of the housing 3152.

Staple holes through which the plurality of staples 3153 may be discharged to the outside may be formed in the cover 3151. The plurality of staples 3153, which were accommodated inside the housing 3152 before a stapling operation, are pushed upward by the working member 3154 during the stapling operation, pass through the staple holes of the cover 3151, and withdrawn out of the cartridge 3150, thereby performing the stapling operation. Meanwhile, a slit may be formed in the cover 3151 in the lengthwise direction.

A blade (not shown) of the working member 3154 may protrude out of the cartridge 3150 through the slit. As the blade (not shown) of the working member 3154 passes along the slit, stapled tissues may be cut. The plurality of staples 3153 may be arranged inside the housing 3152.

As the working member 3154, which will be described later, moves linearly in one direction, the plurality of staples 3153 may be sequentially pushed out of the inside of the housing 3152, thereby performing suturing, that is, stapling. Here, the material constituting the plurality of staples 3153 may include titanium, stainless steel, etc. Meanwhile, a drawing member (not shown) may be further disposed between the housing 3152 and the plurality of staples 3153.

In other words, it may be said that the plurality of staples 3153 are arranged above the drawing member (not shown). In this case, the working member 3154 moves linearly in one direction to push up the drawing member (not shown), and the drawing member (not shown) may push up the plurality of staples 3153. In this way, it may be said that the working member 3154 pushes up the plurality of staples 3153 in both the case where the working member 3154 directly pushes up the plurality of staples 3153 and the case where the working member 3154 pushes up the drawing member (not shown) and the drawing member (not shown) pushes up the plurality of staples 3153 (i.e., the case where the working member 3154 indirectly pushes up the plurality of staples 3153).

The moving member 3155 may be disposed at the bottom of the inside of the housing 3152.

Here, the moving member 3155 is not fixedly coupled to other components of the cartridge 3150 and may be formed to be relatively movable with respect to the other components of the cartridge 3150.

In other words, the moving member 3155 may perform a reciprocating linear motion with respect to the housing 3152 and the cover 3151 coupled to the housing 3152. The working member 3154 may be disposed inside the housing 3152.

The working member 3154 may be formed to be in contact with the moving member 3155 and may be formed to move linearly in one direction according to the reciprocating linear motion of the moving member 3155. In other words, the working member 3154 interacts with the moving member 3155 to perform stapling and cutting while moving in a direction in which a connection portion 3400 extends. Meanwhile, the end tool according to the present embodiment may further include a first staple pulley and a second staple pulley.

The first staple pulley is associated with linear/rotating motions of pulleys and links for stapling and cutting.

Also, the second staple pulley is associated with linear/rotating motions of pulleys and links for stapling and cutting. The first staple pulley and the second staple pulley are formed to face the first pulley 3111 and second pulley 3121, which are end tool jaw pulleys, and are formed to be rotatable independently of each other around the first rotation shaft 3141, which is the rotation shaft of the end tool jaw pulleys. Here, although the drawings show that the first staple pulley and the second staple pulley are arranged between the first pulley 3111 and the second pulley 3121, the technical idea of the present disclosure is not limited thereto, and the first staple pulley and the second staple pulley may be arranged at various locations adjacent to the first pulley 3111 or the second pulley 3121. Here, according to the present disclosure, the first staple pulley, the second staple pulley, the first pulley 3111, and the second pulley 3121 may be formed to rotate around the substantially same axis.

As such, the first staple pulley, the second staple pulley, the first pulley 3111, and the second pulley 3121 may be formed to rotate around the same axis, thereby enabling stapling and cutting motions simultaneously as performing a pitch motion/a yaw motion/an actuation motion. However, although the drawings show that the first staple pulley, the second staple pulley, the first pulley 3111, and the second pulley 3121 are formed to rotate around a single rotation shaft (e.g., the first rotation shaft 3141), pulleys may rotate around different shafts that are concentric with one another. In other words, it may be expressed as a structure in which the first pulley 3111, which is the first jaw pulley, the first staple pulley, the second staple pulley, and the second pulley 3121, which is the second jaw pulley, are sequentially stacked along the first rotation shaft 3141.

Alternatively, it may be expressed as a structure in which the first staple pulley and the second staple pulley are arranged between the first pulley 3111 and the second pulley 3121 facing each other.

Here, the first pulley 3111, which is the first jaw pulley, the first staple pulley, the second staple pulley, and the second pulley 3121, which is the second jaw pulley, may be formed to be rotatable independently of one another. Meanwhile, the end tool 3100 described above is an embodiment of an end tool that may be mounted on the surgical instrument 1000 according to the present disclosure, and the technical idea of the present disclosure is not limited thereto. Some components may be changed, omitted, or added as needed.

Hereinafter, an end tool according to the other embodiment of the present disclosure will be described.

Hereinafter, for convenience of explanation, configurations identical to those of end tools 1100 and 3100 described above or that may be easily modified and employed by one of ordinary skill in the art will be omitted or briefly described.

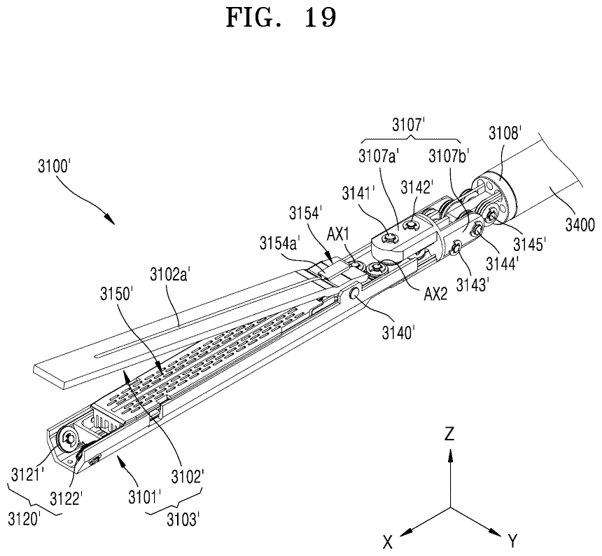

FIG. 19 is a schematic perspective view of an end tool 3100′ according to the other embodiment of the present disclosure.

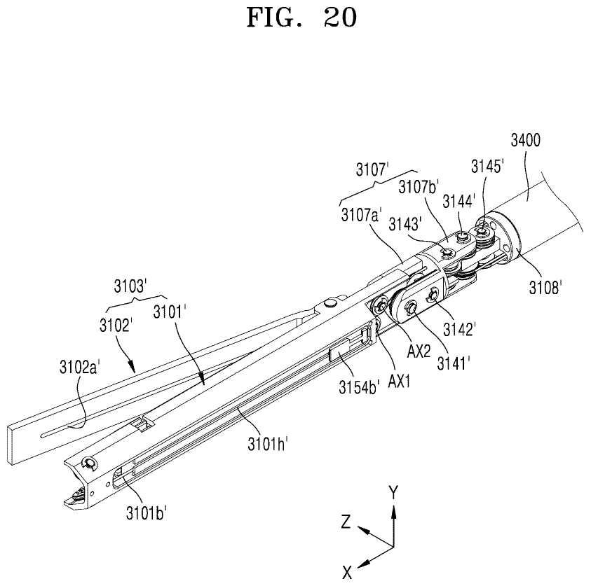

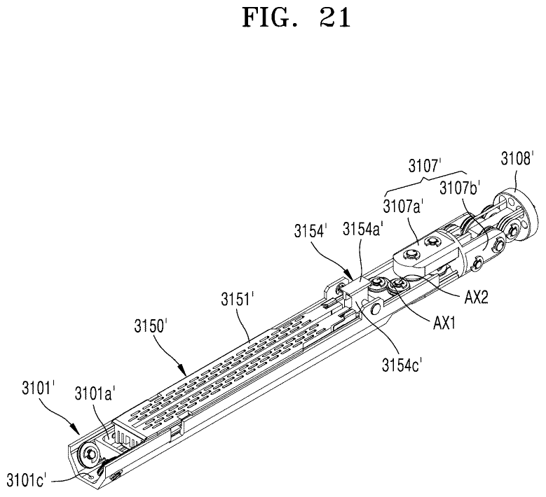

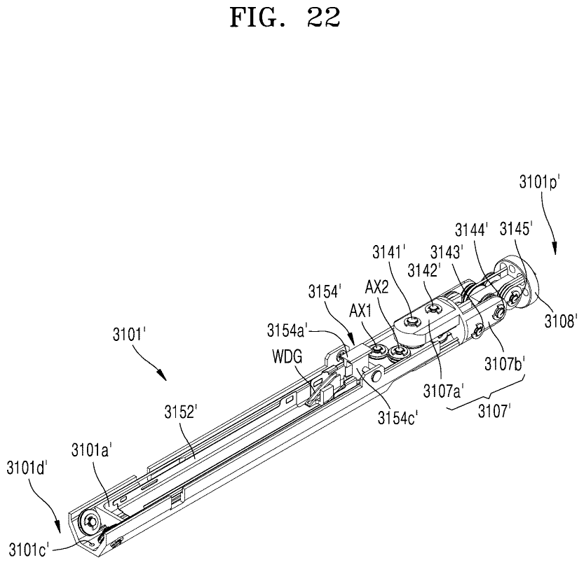

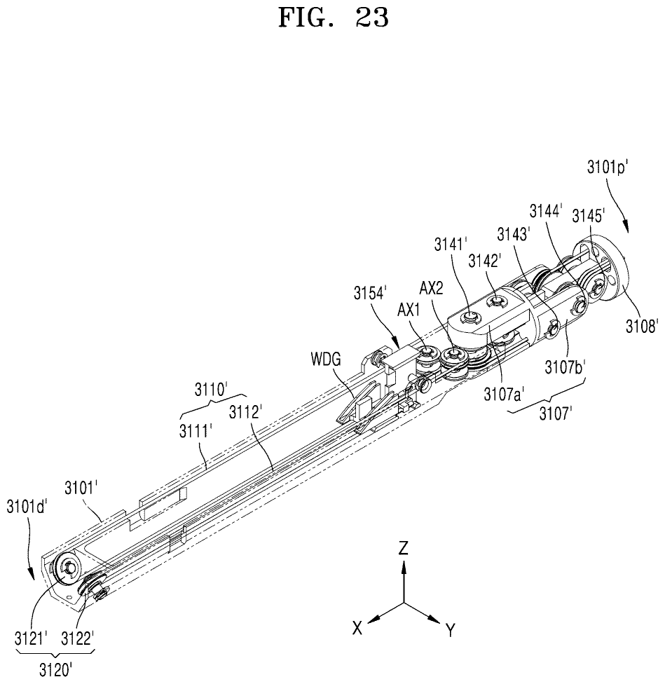

FIG. 20 is a perspective view of the end tool 3100′ of FIG. 19, viewed in another direction. FIG. 21 is a schematic perspective view of the end tool 3100′ of FIG. 19 from which a second jaw 3102′ is removed. FIG. 22 is a schematic perspective view of the end tool 3100′ of FIG. 21 from which a cartridge 3150′ is removed. FIG. 23 is a transparent perspective view of the structure of FIG. 22. Referring to FIGS. 19 to 23, a direction in which an end tool 3100′ according to the present embodiment rotates may be different from directions in which the end tools 1100 and 3100 described above rotate.

For example, in the end tools 1100 and 3100 described above, the actuation rotation shaft of the two jaws 1103 and 3103 may be identical or parallel to the yaw rotation shaft of the end tools 1100 and 3100. However, in the end tool 3100′ according to the present embodiment, the actuation rotation shaft of the two jaws 3103′ may be identical or parallel to the pitch rotation shaft of the end tool 3100′. The end tool 3100′ may include the two jaws 3103′, a plurality of fixed pulleys 3120′, and a plurality of forward wires 3110′.

The plurality of fixed pulleys 3120′ may include two or more pulleys, e.g., a first fixed pulley 3121′ and a second fixed pulley 3122′. The plurality of forward wires 3110′ may include two or more wires, e.g., a first forward wire 3111′ and a second forward wire 3112′. The two jaws 3103′ may perform various functions, such as grip operations, and, according to a specific embodiment, may include a pair of jaws, i.e., a first jaw 3101′ and a second jaw 3102′.

Here, the first jaw 3101′, the second jaw 3102′, or components including the first jaw 3101′ and the second jaw 3102′ may be referred to as two jaws 3103′. The first jaw 3101′ and the second jaw 3102′ may be arranged to face each other and may move toward each other and away from each other. For example, the first jaw 3101′ and the second jaw 3102′ may be formed to rotate around one rotation shaft (e.g., a sixth rotation shaft 3140′).

A cartridge 3150′ may be disposed to be accommodated in the first jaw 3101′, and a plurality of staples (refer to 3153 of FIG. 17, etc.) are arranged inside the cartridge 3150′.

In a state where first jaw 3101′ and second jaw 3102′ are close to each other (e.g., in a state where first jaw 3101′ and second jaw 3102′ are closed with a body tissue therebetween, when a working member 3154′ receives force from the plurality of forward wires 3110′, the working member 3154′ may move in a direction toward a distal end 3101d′ of the first jaw 3101′ and push up the plurality of staples (refer to 3153 in FIG. 17, etc.), thereby performing stapling. At this time, while one or more clamps (3154a′ and 3154b′_of the working member 3154′ are protruding out of the first jaw 3101′ and the second jaw 3102′, the one or more clamps 3154a′ and 3154b′ of the working member 3154′ press the outer surfaces of the first jaw 3101′ and the second jaw 3102′ and move forward, thereby smoothly performing stapling. According to an optional embodiment, the cartridge 3150′ may have a case 3152′ corresponding to the bottom, and the case 3152′ may be disposed at the first jaw 3101′. Meanwhile, the working member 3154′ may be used together with a wedge WDG.

For example, the wedge WDG may be prepared separately from the working member 3154′ and then disposed at the first jaw 3101′ to be adjacent to the working member 3154′. Also, in another example, the working member 3154′ and the wedge WDG may be formed as one body. The wedge WDG may be disposed on at least one side of a main body portion 3154c′ and may be formed to have a predetermined inclined surface. In other words, the wedge WDG may be formed to be inclined to a certain degree in the direction in which the end tool 3100′ extends. In other words, the height of a proximal end 3101p′ of the first jaw 3101′ may be higher than that of a distal end 3101d′. Such the wedge WDG is formed to be able to sequentially contact the drawing member (not shown) or the plurality of staples (3153 of FIG. 17) arranged in the cartridge 3150′ and perform the role of sequentially pushing up the plurality of staples (refer to 3153 of FIG. 17, etc.).

The plurality of fixed pulleys 3120′ may be arranged on the first jaw 3101′ in front of the cartridge 3150′, that is, to be closer to the distal end 3101d′ of the first jaw 3101′ than to the cartridge 3150′.

For example, the plurality of fixed pulleys 3120′ may be arranged disposed in a front space 3101c′ of the first jaw 3101′, and detailed descriptions thereof will be given later. Also, the end tool 3100′ of a surgical instrument according to the present embodiment may include one or more members, e.g., a joint member, connecting the two jaws 3103′ and the connection portion 3400.

Also, according to an optional embodiment, the end tool 3100′ may include an end tool hub 3108′ and a pitch hub 3107′. The end tool hub 3108′ may be disposed to connect the end tool 3100′ and a straight portion of the connection portion 3400.

According to the other embodiment, a fourth rotation shaft 3144′ may correspond to the end tool hub 3108′, and the fourth rotation shaft 3144′ may be a pitch rotation shaft.

In a specific example, the end tool 3100′ may rotate up and down around the fourth rotation shaft 3144′ in the drawing. Also, one or more pulleys may be arranged to be adjacent to the fourth rotation shaft 3144′. The end tool hub 3108′ may be in the form of a long bar that protrudes from the center of a surface corresponding to the connection portion 3400, e.g., a disk-shaped main region, and the fourth rotation shaft 3144′ and a fifth rotation shaft 3145′ may additionally correspond to the bar-like region.

A pitch hub 3107′ is connected to the end tool hub 3108′ and the two jaws 3103′.

The pitch hub 3107′ may be axially coupled to the end tool hub 3108′ along the fourth rotation shaft 3144′. The pitch hub 3107′ may rotate around the fourth rotation shaft 3144′ while being connected to the end tool hub 3108′. In other words, the end tool 3100′ may perform a pitch motion as the pitch hub 3107′ rotates around the fourth rotation shaft 3144′ with respect to the end tool hub 3108′. Also, the two jaws 3103′ of the end tool 3100′ may be axially coupled to the pitch hub 3107′ along the first rotation shaft 3141′.

The two jaws 3103′ may rotate around the first rotation shaft 3141′ while being connected to the pitch hub 3107′. In other words, the two jaws 3103′ of the end tool 3100′ may rotate around the first rotation shaft 3141′ with respect to the pitch hub 3107′ and perform a yaw motion. As a result, the yaw motion of the end tool 3100′ includes the two jaws 3103′ rotating around the first rotation shaft 3141′ with respect to the pitch hub 3107′, and the pitch motion of the end tool 3100′ includes the two jaws 3103′ coupled to the pitch hub 3107′ rotates with the pitch hub 3107′ as the pitch hub 3107′ rotates around the fourth rotation shaft 3144′ with respect to the end tool hub 3108′.

The pitch hub 3107′ may include a first hub 3107a′ and a second hub 3107b′.

The first hub 3107a′ of the pitch hub 3107′ is connected to the two jaws 3103′ and, for example, may be formed long to be connected to one region of the first jaw 3101′. In a specific example, the first hub 3107a′ may include two bar-like regions parallel to each other to face each other, and one region of the first jaw 3101′ may be disposed therebetween and coupled to the two bar-like regions.

The second hub 3107b′ of the pitch hub 3107′ is connected to the end tool hub 3108′ and includes, for example, two bar-like regions parallel to each other to face each other, and one region of the end tool hub 3108′ may be disposed therebetween and coupled to the two bar-like regions.

As described above, the fifth rotation shaft 3145′, which is spaced apart from the fourth rotation shaft 3144′ and is closer to the connection portion 3400 than the fourth rotation shaft 3144′ is, may be disposed at the end tool hub 3108′.

The fourth rotation shaft 3144′ and the fifth rotation shaft 3145′ may include axes in directions parallel to each other. A second rotation shaft 3142′ adjacent to and parallel to the first rotation shaft 3141′ may be disposed at the pitch hub 3107′, and a third rotation shaft 3143′ and the fourth rotation shaft 3144′ extending in a direction different from (e.g., crossing or perpendicular to) the direction of the first rotation shaft 3141′ and the second rotation shaft 3142′ may be sequentially arranged in a direction toward the connection portion 3400 (or a direction away from a working member).

The fourth rotation shaft 3144′ may be a pitch motion axis of the end tool 3100′, and the first rotation shaft 3141′ may be a yaw motion axis of the end tool 3100′.

The third rotation shaft 3143′ and the fifth rotation shaft 3145′ may be pitch auxiliary rotation shafts, and the second rotation shaft 3142′ may be a yaw auxiliary rotation shaft.

At least one region of one or more driving wires, e.g., a wire transmitting driving force of a pitch motion or a wire transmitting driving force of a yaw motion, may be in contact with or wound around the first rotation shaft 3141′, the second rotation shaft 3142′, the third rotation shaft 3143′, the fourth rotation shaft 3144′, and the fifth rotation shaft 3145′. The second rotation shaft 3142′, the third rotation shaft 3143′, and the fifth rotation shaft 3145′ adjacent to the fourth rotation shaft 3144′, which is a pitch motion axis, and the first rotation shaft 3141′, which is a yaw motion axis, may control paths in which the driving wires are wound around the fourth rotation shaft 3144′ and the first rotation shaft 3141′, thereby securing efficiency of the arrangement of the driving wires and stability of power transmission and the paths through the driving wires.

Also, at least one region of the plurality of forward wires 3110′ may be in contact with or wound around the first rotation shaft 3141′, the second rotation shaft 3142′, the third rotation shaft 3143′, the fourth rotation shaft 3144′, and the fifth rotation shaft 3145′.

More detailed descriptions regarding the arrangement of the first rotation shaft 3141′, the second rotation shaft 3142′, the third rotation shaft 3143′, the fourth rotation shaft 3144′, and the fifth rotation shaft 3145′ will be given later.

One or more switching rotation shafts AX1 and AX2 may be arranged at the end tool 3100′, and one or more pulleys corresponding to the switching rotation shafts AX1 and AX2 may be arranged.

For example, the first switching rotation shaft AX1 and the second switching rotation shaft AX2 may be arranged in a direction close to the two jaws 3103′ (specifically, the proximal end 3101p′ of the two jaws 3103′) and may be arranged to be closer to the distal end 3101d′ of the first jaw 3101′ than the above-mentioned rotation shafts (e.g., the first rotation shaft 3141′, the second rotation shaft 3142′, the third rotation shaft 3143′, the fourth rotation shaft 3144′, and the fifth rotation shaft 3145′) are.

The first switching rotation shaft AXI and the second switching rotation shaft AX2 may be shafts formed parallel to each other and are arranged such that their front and rear positions are different from each other. Therefore, the first switching rotation shaft AX1 and the second switching rotation shaft AX2 are sequentially arranged based on the distal end 3101d′ of the first jaw 3101′, wherein portions of the first switching rotation shaft AX1 and the second switching rotation shaft AX2 may overlap each other.

The first switching rotation shaft AX1 and the second switching rotation shaft AX2 are regions that at least portions of the plurality of forward wires 3110′ are wound or in contact with and may organize and guide paths before the plurality of forward wires 3110′ enter the first rotation shaft 3141′, the second rotation shaft 3142′, the third rotation shaft 3143′, the fourth rotation shaft 3144′, and the fifth rotation shaft 3145′.

More detailed descriptions regarding the arrangement of the first switching rotation shaft AX1 and the second switching shaft AX2 will be given later. As shown in FIG. 23, the first forward wire 3111′ and the second forward wire 3112′ are wound around the first fixed pulley 3121′ and the second fixed pulley 3122′ to change paths, are connected beyond the end tool 3100′ through at least portions of the rotation shafts (e.g., the first rotation shaft 3141′, the second rotation shaft 3142′, the third rotation shaft 3143′, the fourth rotation shaft 3144′, and the fifth rotation shaft 3145′), and may be precisely controlled by being further connected to a manipulation portion through the connection portion 3400.

Through this, precise motion control of the working member 3154′ may be easily implemented, and detailed descriptions thereof will be given later. The two jaws 3103′ of the end tool 3100′ will be described in more detail.

The second jaw 3102′ is formed to have an overall shape of a long bar. For example, the second jaw 3102′ may be formed in a bar-like shape to include at least one region corresponding to the first jaw 3101′.

The proximal end of second jaw 3102′ may include a region coupled to the first jaw 3101′.

According to an embodiment, the second jaw 3102′ is formed to be rotatable with respect to the first jaw 3101′ around the sixth rotation shaft 3140′ of the proximal end. The second jaw 3102′ may have various forms. As a specific example, a plurality of anvil grooves are formed on at least one region of a surface of the second jaw 3102′ facing the first jaw 3101′ from among surfaces of the second jaw 3102′, wherein such an anvil groove may have a shape corresponding to that of a staple (refer to 3153 of FIG. 17, etc.).

The plurality of anvil grooves of the second jaw 3102′ may serve as a support for bending the plurality of staples (refer to 3153, etc. of FIG. 17) when the working member 3154′ pushes up the plurality of staples (refer to 3153, etc. of FIG. 17) during a staple operation.

The second jaw 3102′ includes a guide groove 3102a′.

The guide groove 3102a′ may have a long shape extending in the lengthwise direction of the second jaw 3102′. The guide groove 3102a′ may be formed to guide the working member 3154′ and may be a groove penetrating through a region facing the working member 3154′.

Therefore, one region of the working member 3154′, e.g., at least one region of a main body portion 3154c′ of the working member 3154′ or a first clamp 3154a′ connected thereto, may pass through the guide groove 3102a′ and be discharged out of the second jaw 3102′. When the working member 3154′ moves forward, the first clamp 3154a′ passes through the guide groove 3102a′ of the second jaw 3102′, is exposed to the outside of the second jaw 3102′, and may contact or press the top surface of the second jaw 3102′. Through the movement of the working member 3154′, the first clamp 3154a′ presses the top surface of the second jaw 3102′, and a second clamp 3154b′, which will be described later, presses the bottom surface of the first jaw 3101′. As a result, the gap between the second jaw 3102′ and the first jaw 3101′ is narrowed, and thus a state in which the second jaw 3102′ is closed with respect to the first jaw 3101′ may be naturally maintained. The first jaw 3101′ is formed to have an overall shape of a long bar, a rotation shaft may be disposed at the proximal end to enable rotational motion, and the rotation shaft may correspond to the sixth rotation shaft 3140′ formed in the above-stated second jaw 3102′.

Also, the cartridge 3150′ may be accommodated in a portion of the first jaw 3101′ closer to the distal end 3101d′ than to the rotation shaft. For example, the first jaw 3101′ is formed in the form of an overall hollow box with one side (top surface) removed, and a cartridge accommodation portion 3101a′ capable of accommodating the cartridge 3150′ may be formed inside the first jaw 3101′.

In other words, the first jaw 3101′ may have a cross-section having an approximately a ‘U’-like shape. A guide groove 3101h′ may be formed on the bottom surface of the first jaw 3101′, that is, the bottom surface facing the open side from which the top surface is removed.

In detail, the guide groove 3101h′ may be formed to guide the linear movement of the working member 3154′. The guide groove 3101h′ may be formed to guide the working member 3154′ and may be a groove penetrating through a region facing the working member 3154′.

Therefore, one region of the working member 3154′, e.g., at least one region of a main body portion 3154c′ of the working member 3154′ or a second clamp 3154b′ connected thereto, may pass through the guide groove 3101h′ and be discharged out of the first jaw 3101′. When the working member 3154′ moves forward, the second clamp 3154b′ passes through the guide groove 3101h′ of the first jaw 3101′, is exposed to the outside of the first jaw 3101′, and may contact or press the top surface of the first jaw 3101′. Through the movement of the working member 3154′, the second clamp 3154b′ presses the bottom surface of the first jaw 3101′, and the first clamp 3154a′ presses the top surface of the first jaw 3101′. As a result, the gap between the second jaw 3102′ and the first jaw 3101′ is narrowed, and thus a state in which the second jaw 3102′ is closed with respect to the first jaw 3101′ may be naturally maintained. According to an optional embodiment, the first jaw 3101′ may include a window 3101b′.

After the operation of the working member 3154′ or the end tool 3100′ is used, the second clamp 3154b′ of the working member 3154′ may correspond to the window 3101b′ and the coupling between the first jaw 3101′ and the working member 3154′ may be released. The first jaw 3101′ may include the front space 3101c′ ahead of the cartridge accommodation portion 3101a′.

For example, the front space 3101c′ may be disposed closer to the distal end 3101d′ of the first jaw 3101′ than to the cartridge accommodation portion 3101a′.

The plurality of fixed pulleys 3120′ may be arranged in the front space 3101c′. For example, the first fixed pulley 3121′ and the second fixed pulley 3122′ may be disposed. Meanwhile, the end tool 3100′ described above is an embodiment of an end tool that may be mounted on the surgical instrument 1000 according to the present disclosure, and the technical idea of the present disclosure is not limited thereto. Some components may be changed, omitted, or added as needed.

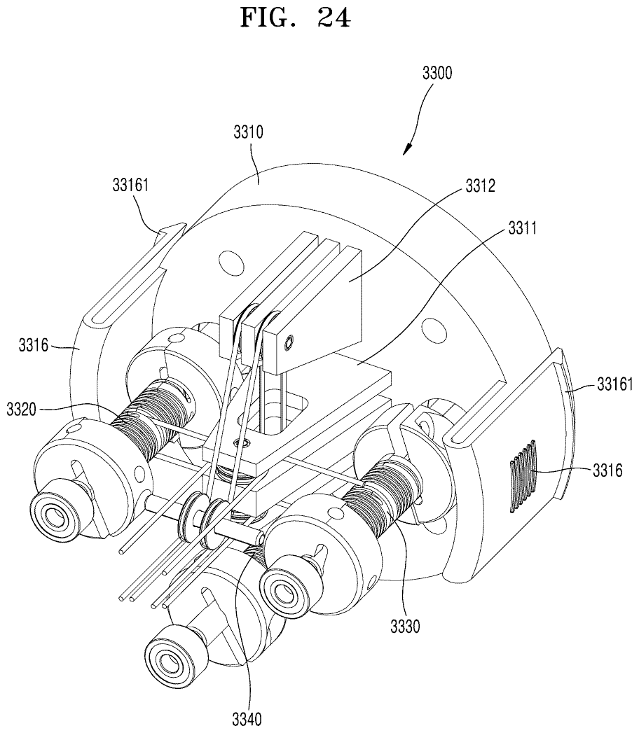

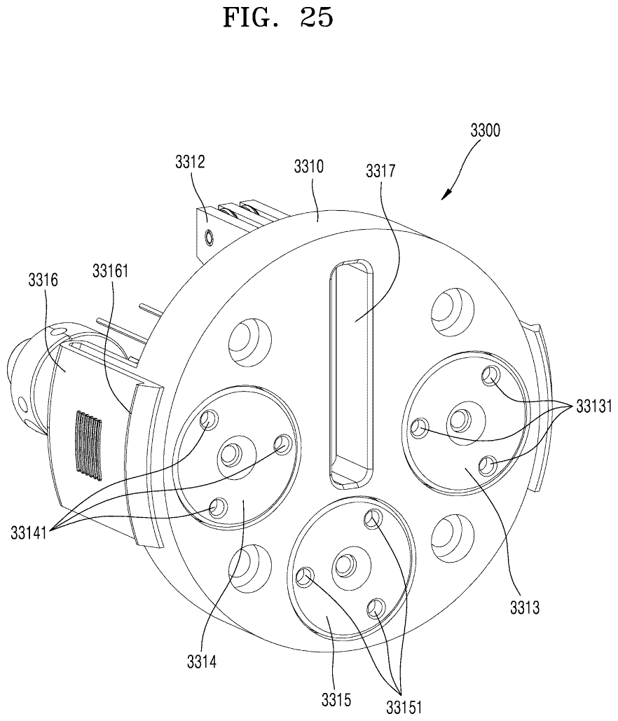

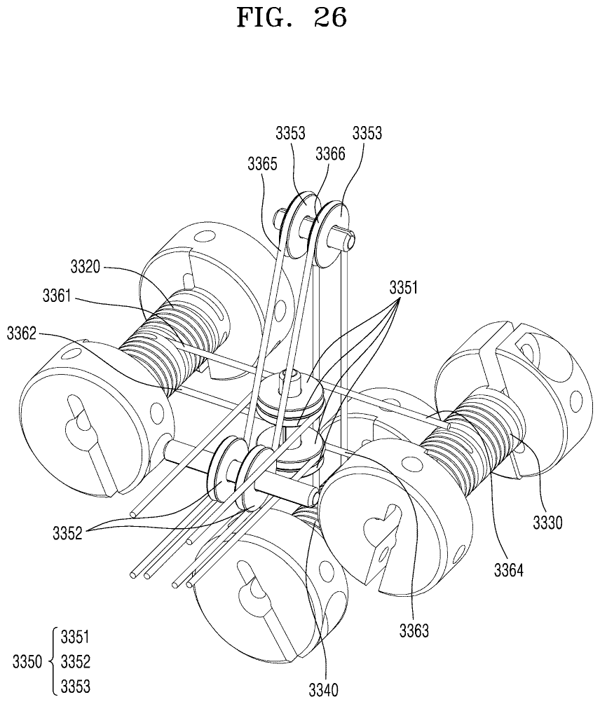

FIG. 24 is a diagram for describing the internal structure of a power transmission portion 3300 according to the other embodiment of the present disclosure, FIG. 25 is a rear view of the power transmission portion 3300 of FIG. 24, and FIG. 26 is a diagram for describing the arrangement of pulleys and wires of the power transmission portion 3300 of FIG. 24.

Referring to FIGS. 24 to 26, a power transmission portion 3300 according to the other embodiment of the present disclosure may include a pulley frame 3310, at least one pulley, and at least one wire.

The pulley frame 3310 may form the overall framework of the power transmission portion 3300.

At least one pulley may be disposed in the pulley frame 3310.

Here, disposing a pulley should be interpreted in a broad sense. For example, disposing a pulley may mean that the pulley is directly connected to the pulley frame 3310 or may mean that a rotation shaft is installed in the pulley frame 3310 and the pulley is connected to the rotation shaft. Alternatively, disposing a pulley may mean that a separate member is provided in the pulley frame 3310, a rotation shaft is connected to the separate member, and the pulley is connected to the rotation shaft. Alternatively, disposing a pulley may mean that a hole is formed in the pulley frame 3310, a rotation shaft is disposed to pass through the hole, and a pulley is installed on the rotation shaft. The power transmission portion 3300 may include at least one pulley.

A pulley is a member around which a wire is wound, and a groove in which a wire may be wound may be formed on the pulley.

According to an embodiment, the power transmission portion 3300 may include a yaw pulley 3320, a pitch pulley 3330, and a firing pulley 3340.

Also, the power transmission portion 3300 may include at least one yaw wire, at least one pitch wire, and firing wires. The yaw pulley 3320 is a pulley associated with the yaw rotation of the end tool 3100, and the first yaw wire 3361 and the second yaw wire 3362 are wires associated with the yaw rotation of the end tool 3100.

The yaw pulley 3320 may be disposed in one region of the pulley frame 3310.

The first yaw wire 3361 and the second yaw wire 3362 may be arranged at the end tool 3100 and extend from a pulley associated with the yaw rotation of the end tool 3100 toward the yaw pulley 3320.

According to an embodiment, the first yaw wire 3361 and the second yaw wire 3362 may be a pair of yaw wires.

As described later, the first yaw wire 3361 and the second yaw wire 3362 may be connected to the upper portion and the lower portion of the yaw pulley 3320, respectively. According to a specific embodiment, the yaw wires may include a first yaw wire 3361 and a second yaw wire 3362.

In FIG. 26, the first yaw wire 3361 may extend to the upper portion of the yaw pulley 3320 and be connected to the yaw pulley 3320. In FIG. 26, the second yaw wire 3362 may extend to the lower portion of the yaw pulley 3320 and be connected to the yaw pulley 3320. After the first yaw wire 3361 and the second yaw wire 3362 are connected to the yaw pulley 3320, the first yaw wire 3361 and the second yaw wire 3362 may be wound around or unwound from the yaw pulley 3320 when the yaw pulley 3320 rotates.

In other words, when the yaw pulley 3320 rotates in one direction, one yaw wire is wound around the yaw pulley 3320, and the other one yaw wire is unwound from the yaw pulley 3320. For example, when the yaw pulley 3320 rotates clockwise in FIG. 26, the first yaw wire 3361 is unwound from the yaw pulley 3320, and the second yaw wire 3362 is wound around the yaw pulley 3320. On the contrary, when the yaw pulley 3320 rotates counterclockwise in FIG. 26, the first yaw wire 3361 is wound around the yaw pulley 3320, and the second yaw wire 3362 is unwound from the yaw pulley 3320. In other words, it may be said that, when the yaw pulley 3320 rotates, the pair of yaw wires 3361 and 3362 move in opposite directions around the yaw pulley 3320.

In this regard, when the yaw pulley 3320 rotates and moves the pair of the first yaw wire 3361 and the second yaw wire 3362 in different directions, a pulley connected to the end tool 3100 through the first yaw wire 3361 and the second yaw wire 3362 rotates in a direction corresponding to the directions in which the pair of the first yaw wire 3361 and the second yaw wire 3362 are moved.

Therefore, the pulley connected to the end tool 3100 through the first yaw wire 3361 and the second yaw wire 3362 may implement yaw rotation of the end tool 3100 while rotating in one direction. The pitch pulley 3330 is a pulley associated with the pitch rotation of the end tool 3100, and a first pitch wire 3363 and a second pitch wire 3364 are wires associated with the pitch rotation of the end tool 3100.

The pitch pulley 3330 may be disposed in one region of the pulley frame 3310.

According to an embodiment, the pitch pulley 3330 may be disposed on a side opposite to the yaw pulley 3320.

According to a preferred embodiment, the pitch pulley 3330 and the yaw pulley 3320 may be arranged symmetrically on both sides of the pulley frame 3310 around the center of the pulley frame 3310. Therefore, as described later, the first yaw wire 3361 and the second yaw wire 3362, and the first pitch wire 3363 and the second pitch wire 3364 extending through an auxiliary pulley 3350 may approach to the yaw pulley 3320 and the pitch pulley 3330 at almost vertical angles, respectively. The first pitch wire 3363 and the second pitch wire 3364 may be arranged at the end tool 3100 and extend from a pulley associated with the pitch rotation of the end tool 3100 toward the pitch pulley 3330.

According to an embodiment, the first pitch wire 3363 and the second pitch wire 3364 may be a pair of pitch wires.

As described later, the first pitch wire 3363 and the second pitch wire 3364 may be connected to the upper portion and the lower portion of the pitch pulley 3330, respectively. As a specific example, the pitch wires may include a first pitch wire 3363 and a second pitch wire 3364.

In FIG. 26, the first pitch wire 3363 may extend to the lower portion of the pitch pulley 3330 and be connected to the pitch pulley 3330. In FIG. 26, the second pitch wire 3364 may extend to the upper portion of the pitch pulley 3330 and be connected to the pitch pulley 3330. After the first pitch wire 3363 and the second pitch wire 3364 are connected to the pitch pulley 3330, the first pitch wire 3363 and the second pitch wire 3364 may be wound around or unwound from the pitch pulley 3330 when the pitch pulley 3330 rotates.

In other words, when the pitch pulley 3330 rotates in one direction, one pitch wire is wound around the pitch pulley 3330, and the other one pitch wire is unwound from the pitch pulley 3330. For example, when the pitch pulley 3330 rotates clockwise in FIG. 26, the first pitch wire 3363 is unwound from the pitch pulley 3330, and the second pitch wire 3364 is wound around the pitch pulley 3330. On the contrary, when the pitch pulley 3330 rotates counterclockwise in FIG. 26, the first pitch wire 3363 is wound around the pitch pulley 3330, and the second pitch wire 3364 is unwound from the pitch pulley 3330. In other words, it may be said that, when the pitch pulley 3330 rotates, the pair of pitch wires 3363 and 3364 move in opposite directions around the pitch pulley 3330.

In this regard, when the pitch pulley 3330 rotates and moves the pair of the first pitch wire 3363 and the second pitch wire 3364 in different directions, a pulley connected to the end tool 3100 through the first pitch wire 3363 and the second pitch wire 3364 rotates in a direction corresponding to the directions in which the pair of the first pitch wire 3363 and the second pitch wire 3364 are moved.

Therefore, the pulley connected to the end tool 3100 through the first pitch wire 3363 and the second pitch wire 3364 may implement pitch rotation of the end tool 3100 while rotating in one direction. The firing pulley 3340 is a pulley associated with the movement of the working member 3154 provided at the end tool 3100, and a first firing wire 3365 and a second firing wire 3366 are wires associated with the movement of the working member 3154 provided at the end tool 3100.

The firing pulley 3340 may be disposed in one region of the pulley frame 3310.

According to a specific embodiment, the firing pulley 3340 may be disposed at a position horizontally different from those of the yaw pulley 3320 and the pitch pulley 3330 of the pulley frame 3310. For example, the firing pulley 3340 may be disposed at a position lower than the yaw pulley 3320 and the pitch pulley 3330.

For example, as shown in FIG. 24, the yaw pulley 3320 and the pitch pulley 3330 may be arranged at the pulley frame 3310 and may be arranged side-by-side with each other, and the firing pulley 3340 may be disposed at a position lower than the yaw pulley 3320 and the pitch pulley 3330. According to the other embodiment, the first firing wire 3365 and the second firing wire 3366 may be connected to the moving member 3155 of the end tool 3100. In this case, the first firing wire 3365 and the second firing wire 3366 may extend from the moving member 3155 toward the firing pulley 3340.

According to another embodiment, the first firing wire 3365 and the second firing wire 3366 may be directly connected to the working member 3154. In this case, the first firing wire 3365 and the second firing wire 3366 may extend from the working member 3154 toward the firing pulley 3340. According to another embodiment, when a pulley associated with a translational motion of the working member 3154 is separately provided in the end tool 3100, the first firing wire 3365 and the second firing wire 3366 may be connected to the pulley, and the first firing wire 3365 and the second firing wire 3366 may extend from the pulley toward the firing pulley 3340. According to the other embodiment, the first firing wire 3365 and the second firing wire 3366 may be a pair of firing wires.

As described later, the first firing wire 3365 and the second firing wire 3366 may be connected to the left portion and the right portion of the firing pulley 3340, respectively. According to a specific embodiment, the firing wires may include a first firing wire 3365 and a second firing wire 3366.

In FIG. 26, the first firing wire 3365 may extend to the left of the firing pulley 3340 and be connected to the firing pulley 3340. In FIG. 26, the second firing wire 3366 may extend to the right of the firing pulley 3340 and be connected to the firing pulley 3340. After the first firing wire 3365 and the second firing wire 3366 are connected to the firing pulley 3340, the first firing wire 3365 and the second firing wire 3366 may be wound around or unwound from the firing pulley 3340 when the firing pulley 3340 rotates.

In other words, when the firing pulley 3340 rotates in one direction, one firing wire is wound around the firing pulley 3340, and the other one firing wire is unwound from the firing pulley 3340. For example, when the firing pulley 3340 rotates clockwise in FIG. 26, the first firing wire 3365 is unwound from the firing pulley 3340, and the second firing wire 3366 is wound around the firing pulley 3340. On the contrary, when the firing pulley 3340 rotates counterclockwise in FIG. 26, the first firing wire 3365 is wound around the firing pulley 3340, and the second firing wire 3366 is unwound from the firing pulley 3340. In other words, it may be said that, when the firing pulley 3340 rotates, the pair of the first firing wire 3365 and the second firing wire 3366 move in opposite directions around the firing pulley 3340.

In this regard, when the firing pulley 3340 rotates to move the pair of the first firing wire 3365 and the second firing wire 3366 in different directions, the working member 3154 provided at the end tool 3100 may move forward or backward in correspondence thereto.

According to the other embodiment, when the working member 3154 is formed to move dependently by the movement of the moving member 3155, the pair of the first firing wire 3365 and the second firing wire 3366 may move the moving member 3155 forward or backward as the firing pulley 3340 rotates, and thus the working member 3154 may move forward or backward. According to another embodiment, when the working member 3154 is formed to be connected to the first firing wire 3365 and the second firing wire 3366 and directly move forward or backward, as the firing pulley 3340 rotates, the pair of the first firing wire 3365 and the second firing wire 3366 may directly move the working member 3154 forward or backward. However, the present disclosure is not limited thereto. When the end tool 3100 is formed to have a separate pulley associated with the translational motion of the working member 3154, the first firing wire 3365 and the second firing wire 3366 may be connected to the pulley and rotate the pulley to move the working member 3154 forward or backward. According to an embodiment, the power transmission portion 3300 may further include at least one auxiliary pulley 3350.

The auxiliary pulley 3350 may serve to change paths of wires entering the power transmission portion 3300. According to an embodiment, the auxiliary pulley 3350 may include a first auxiliary pulley 3351 that is connected to the first yaw wire 3361, the second yaw wire 3362, the first pitch wire 3363 and the second pitch wire 3364, and changes paths of the first yaw wire 3361, the second yaw wire 3362, the first pitch wire 3363 and the second pitch wire 3364.

Also, the auxiliary pulley 3350 may include a second auxiliary pulley 3352 and a third auxiliary pulley 3353 that are connected to the first firing wire 3365 and the second firing wire 3366 and change paths of the first firing wire 3365 and the second firing wire 3366. The first auxiliary pulley 3351 is disposed at the power transmission portion 3300 and may play a role of changing the paths of the first yaw wire 3361, the second yaw wire 3362, the first pitch wire 3363 and the second pitch wire 3364 extending from the end tool 3100 to the power transmission portion 3300.

A first auxiliary pulley fixing portion 3311 may be formed at the pulley frame 3310.

The first auxiliary pulley fixing portion 3311 is a portion in which the first auxiliary pulley 3351 is installed. For example, the first auxiliary pulley fixing portion 3311 may be formed as one body with the pulley frame 3310. Alternatively, the first auxiliary pulley fixing portion 3311 may be formed as a separate member and may be coupled or assembled to the pulley frame 3310. According to the other embodiment, the first auxiliary pulley fixing portion 3311 may include at least one through hole, and the rotation shaft of the first auxiliary pulley 3351 may be disposed to pass through the through hole.

In other words, the first auxiliary pulley 3351 may be disposed to overlap the through hole formed in the first auxiliary pulley fixing portion 3311, and the rotation shaft may be disposed to simultaneously penetrate through the first auxiliary pulley 3351 and the first auxiliary pulley fixing portion 3311. The first auxiliary pulley fixing portion 3311 may be formed in a shape that at least partially extends from the pulley frame 3310 in a direction toward the connection portion 3400.

For example, the first auxiliary pulley fixing portion 3311 may be formed to extend from one surface of the pulley frame 3310 in a direction toward the connection portion 3400. According to a preferred embodiment, the first auxiliary pulley fixing portion 3311 may be disposed at the center of the pulley frame 3310. In this case, the yaw pulley 3320 and the pitch pulley 3330 may be arranged on both sides of the first auxiliary pulley fixing portion 3311, respectively.

According to a preferred embodiment, the yaw pulley 3320 and the pitch pulley 3330 may be arranged at locations symmetrical around the first auxiliary pulley fixing portion 3311. In other words, the first yaw wire 3361, the second yaw wire 3362, the first pitch wire 3363 and the second pitch wire 3364 entering the first auxiliary pulley 3351 may be distributed to both sides while passing through the first auxiliary pulley fixing portion 3311. Therefore, it may be said that the first yaw wire 3361 and the second yaw wire 3362 extend toward the yaw pulley 3320, and the first pitch wire 3363 and the second pitch wire 3364 extend toward the pitch pulley 3330. Therefore, the first yaw wire 3361 and the second yaw wire 3362 may approach to the yaw pulley 3320 at almost vertical angles, and the first pitch wire 3363 and the second pitch wire 3364 may approach to the pitch pulley 3330 at almost vertical angles.

Preferably, the first yaw wire 3361 and the second yaw wire 3362 may approach to the yaw pulley 3320 vertically, and the first pitch wire 3363 and the second pitch wire 3364 may approach to the pitch pulley 3330 vertically. In other words, the first yaw wire 3361 and the second yaw wire 3362 may approach to the yaw pulley 3320 to form a tangent to the yaw pulley 3320, and the first pitch wire 3363 and the second pitch wire 3364 may approach to the pitch pulley 3330 to form a tangent to the pitch pulley 3330. The first auxiliary pulley 3351 is disposed on the first auxiliary pulley fixing portion 3311 and may change the paths of the first yaw wire 3361, the second yaw wire 3362, the first pitch wire 3363 and the second pitch wire 3364 extending to the power transmission portion 3300.

In detail, the first auxiliary pulley 3351 may be disposed between the yaw pulley 3320 and the pitch pulley 3330, as shown in FIG. 26.

A plurality of first auxiliary pulleys 3351 may be provided.

The number of first auxiliary pulleys 3351 may at least correspond to the number of wires entering the power transmission portion 3300. For example, the yaw wires may include a pair of the first yaw wire 3361 and the second yaw wire 3362 to enter the upper portion and the lower portion of the yaw pulley 3320, respectively.

Also, the pitch wires may include a pair of the first pitch wire 3363 and the second pitch wire 3364 to enter the upper portion and the lower portion of the pitch pulley 3330, respectively. In other words, when four wires entering the power transmission portion 3300 are provided, four first auxiliary pulleys 3351 may be provided. According to an optional embodiment, the plurality of first auxiliary pulleys 3351 may be arranged side-by-side.

For example, the plurality of first auxiliary pulleys 3351 may be arranged parallel to one another. This may be for wires passing through the first auxiliary pulley 3351 extend to the yaw pulley 3320 and the pitch pulley 3330 in parallel or almost parallel with each other. In detail, wires extending from the end tool 3100 to the power transmission portion 3300 may extend in parallel with one another.

Therefore, since the plurality of first auxiliary pulleys 3351 are arranged in parallel with one another, wires passing through the first auxiliary pulley 3351 may extend to the yaw pulley 3320 and/or the pitch pulley 3330 in parallel or almost parallel with one another. According to an optional embodiment, the first auxiliary pulley 3351 may be disposed, such that a direction in which wires are wound around the first auxiliary pulley 3351 and directions in which the wires are wound around the yaw pulley 3320 and the pitch pulley 3330 are almost perpendicular to each other, and preferably, are perpendicular to each other.

In detail, with reference to FIG. 26, a direction in which the wires are wound around the first auxiliary pulley 3351 and directions in which the wires are wound around the yaw pulley 3320 and the pitch pulley 3330 may be perpendicular to each other. In other words, it may be said that the first auxiliary pulley 3351 changes directions in which the wires entering the first auxiliary pulley 3351 extend by 90°. In other words, it may be said that a groove formed on the first auxiliary pulley 3351 and grooves formed on the yaw pulley 3320 and the pitch pulley 3330 are perpendicular to each other. By this configuration, paths of the wires entering the power transmission portion 3300 are changed through the first auxiliary pulley 3351, and the wires may approach toward the yaw pulley 3320 and the pitch pulley 3330 vertically or at almost vertical angles.

The second auxiliary pulley 3352 is disposed at the power transmission portion 3300 and may play a role of changing the paths of the firing wires 3365 and 3366 extending from the end tool 3100 to the power transmission portion 3300.

The third auxiliary pulley 3353 may be disposed at the power transmission portion 3300 and may play a role of changing the paths of the first firing wire 3365 and the second firing wire 3366 extending from the second auxiliary pulley 3352.

Referring again to FIG. 24, the second auxiliary pulley 3352 may guide the first firing wire 3365 and the second firing wire 3366 entering the power transmission portion 3300 from the end tool 3100 through the connection portion 3400 upward.

In detail, the second auxiliary pulley 3352 may change the paths of the first firing wire 3365 and the second firing wire 3366 entering the power transmission portion 3300 to guide the first firing wire 3365 and the second firing wire 3366 in a direction opposite to a direction in which the firing pulley 3340 is located. The third auxiliary pulley 3353 may change the paths of the first firing wire 3365 and the second firing wire 3366 extending from the second auxiliary pulley 3352 to guide the first firing wire 3365 and the second firing wire 3366 toward the firing pulley 3340. Therefore, the first firing wire 3365 and the second firing wire 3366 may be extended in a direction away from the firing pulley 3340 by the second auxiliary pulley 3352 and then extended back toward the firing pulley 3340.

As described above, the firing pulley 3340 and the first firing wire 3365 and the second firing wire 3366 are associated with the movement of the working member 3154 of the end tool 3100.

At this time, the first firing wire 3365 and the second firing wire 3366 may be wound around the firing pulley 3340 for a number of times greater than the number of times the first yaw wire 3361 and the second yaw wire 3362 or the first pitch wire 3363 and the second pitch wire 3364 are wound. This is associated with the movement of the working member 3154. For example, lengths of the first firing wire 3365 and the second firing wire 3366 wound or unwound for moving the working member 3154 are greater than lengths of the first yaw wire 3361, the second yaw wire 3362, the first pitch wire 3363 and the second pitch wire 3364 wound or unwound for a yaw motion and a pitch motion of the end tool 3100. At this time, since the groove formed in the firing pulley 3340 is formed in the shape of a screw hole, the more times the first firing wire 3365 and the second firing wire 3366 are wound around the firing pulley 3340, the more the first firing wire 3365 and the second firing wire 3366 moves in the axial direction of the firing pulley 3340.

This means that the angle of the first firing wire 3365 and the second firing wire 3366 extending from the third auxiliary pulley 3353 to the firing pulley 3340 increases (connected obliquely). In this case, relatively strong stress may be applied to the first firing wire 3365 and the second firing wire 3366. Therefore, as the second auxiliary pulley 3352 guides the first firing wire 3365 and the second firing wire 3366 in a direction opposite to the firing pulley 3340 and the third auxiliary pulley 3353 guides the first firing wire 3365 and the second firing wire 3366 back in a direction toward the firing pulley 3340, lengths by which the first firing wire 3365 and the second firing wire 3366 extend from the third auxiliary pulley 3353 to the firing pulley 3340 may be secured. As a result, the changes in angles at which the first firing wire 3365 and the second firing wire 3366 enter the firing pulley 3340 may be reduced.

A second auxiliary pulley fixing portion 3312 may be formed at the pulley frame 3310.

The second auxiliary pulley fixing portion 3312 is a portion in which the third auxiliary pulley 3353 is installed. For example, the second auxiliary pulley fixing portion 3312 may be formed as one body with the pulley frame 3310. Alternatively, the second auxiliary pulley fixing portion 3312 may be formed as a separate member and may be coupled or assembled to the pulley frame 3310. According to the other embodiment, the second auxiliary pulley fixing portion 3312 may include at least one through hole, and the rotation shaft of the second auxiliary pulley 3352 may be disposed to pass through the through hole.

In other words, the third auxiliary pulley 3353 may be disposed to overlap the through hole formed in the second auxiliary pulley fixing portion 3312, and the rotation shaft may be disposed to simultaneously penetrate through the third auxiliary pulley 3353 and the second auxiliary pulley fixing portion 3312. The second auxiliary pulley fixing portion 3312 may be formed in a shape that protrudes from the pulley frame 3310 in a direction toward the connection portion 3400.

For example, the second auxiliary pulley fixing portion 3312 may be formed to protrude from one surface of the pulley frame 3310 in a direction toward the connection portion 3400. According to a preferred embodiment, the second auxiliary pulley fixing portion 3312 may be disposed on the upper portion of the pulley frame 3310. For example, the second auxiliary pulley fixing portion 3312 may be disposed on a side opposite to the firing pulley 3340 based on the center of the pulley frame 3310. According to the other embodiment, the second auxiliary pulley 3352 and the third auxiliary pulley 3353 may each be provided as a pair.

For example, the second auxiliary pulley 3352 may include a pulley to which the first firing wire 3365 is connected and a pulley to which the second firing wire 3366 is connected. Also, the third auxiliary pulley 3353 may include a pulley to which the first firing wire 3365 is connected and a pulley to which the second firing wire 3366 is connected. According to an optional embodiment, two second auxiliary pulleys 3352 may be arranged parallel to each other.

This may be for the first firing wire 3365 and the second firing wire 3366 passing through the second auxiliary pulley 3352 extend to the third auxiliary pulley 3353 in parallel or almost parallel with each other. In detail, the first firing wire 3365 and the second firing wire 3366 extending from the end tool 3100 to the power transmission portion 3300 extend in parallel with each other.

Therefore, since two second auxiliary pulleys 3352 are arranged in parallel with one another, the first firing wire 3365 and the second firing wire 3366 passing through the second auxiliary pulley 3352 may extend to the third auxiliary pulley 3353 in parallel or almost parallel with each other. According to an optional embodiment, the two third auxiliary pulleys 3353 may be arranged parallel to one another.

This may be for the first firing wire 3365 and the second firing wire 3366 passing through the third auxiliary pulley 3353 extend to the firing pulley 3340 in parallel or almost parallel with each other. In detail, the first firing wire 3365 and the second firing wire 3366 extending from the second auxiliary pulley 3352 to the third auxiliary pulley 3353 extend in parallel with each other.

Therefore, since the two third auxiliary pulleys 3353 are arranged in parallel with each other, the first firing wire 3365 and the second firing wire 3366 passing through the third auxiliary pulley 3353 may extend to the firing pulley 3340 in parallel or almost parallel with each other. According to an optional embodiment, the second auxiliary pulley 3352 and the third auxiliary pulley 3353 may be arranged, such that a direction in which the first firing wire 3365 and the second firing wire 3366 are wound around the second auxiliary pulley 3352 and the third auxiliary pulley 3353 and directions in which the first firing wire 3365 and the second firing wire 3366 are wound around the firing pulley 3340 are almost perpendicular to each other, and preferably, are perpendicular to each other.

In detail, with reference to FIG. 24, directions in which the first firing wire 3365 and the second firing wire 3366 are wound around the second auxiliary pulley 3352 and the third auxiliary pulley 3353 and a direction in which the first firing wire 3365 and the second firing wire 3366 are wound around the firing pulley 3340 may be perpendicular to each other. In other words, it may be said that a groove formed on the first auxiliary pulley 3351 and grooves formed on the yaw pulley 3320 and the pitch pulley 3330 are perpendicular to each other. By this configuration, paths of the first firing wire 3365 and the second firing wire 3366 passing through the second auxiliary pulley 3352 and the third auxiliary pulley 3353, and the first firing wire 3365 and the second firing wire 3366 may approach toward the firing pulley 3340 vertically or at almost vertical angles.

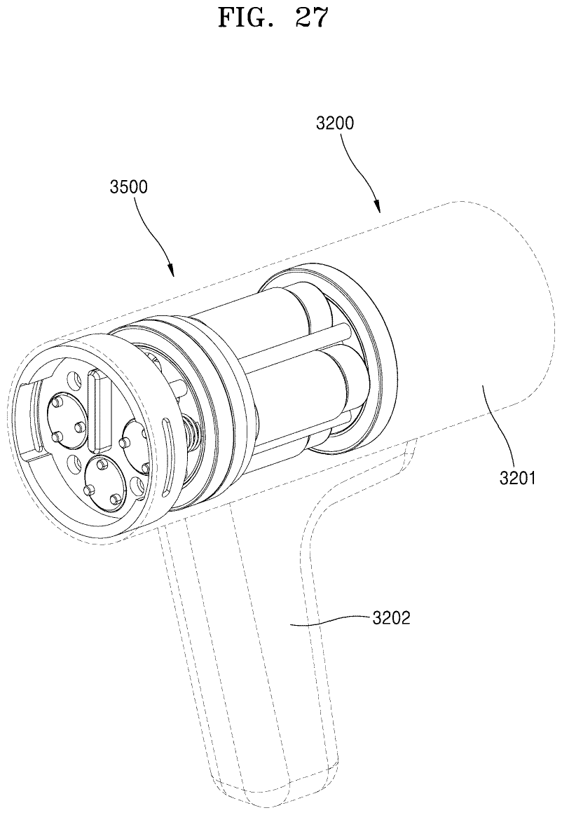

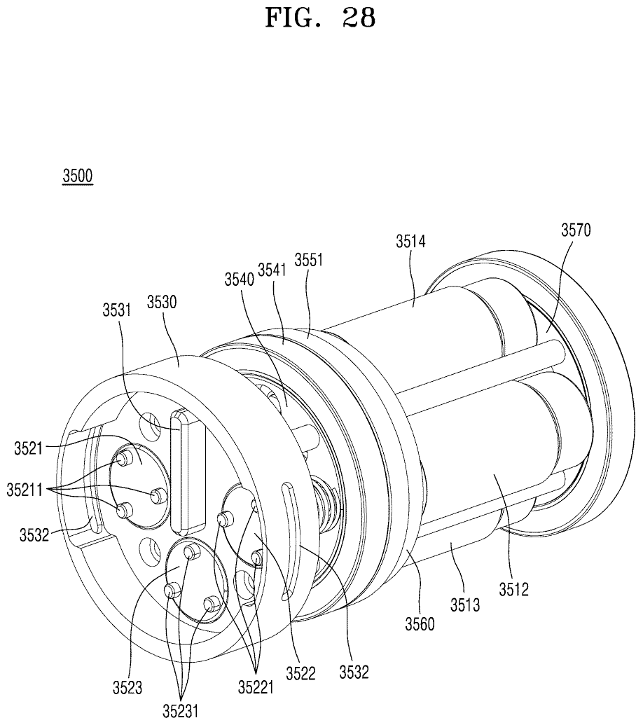

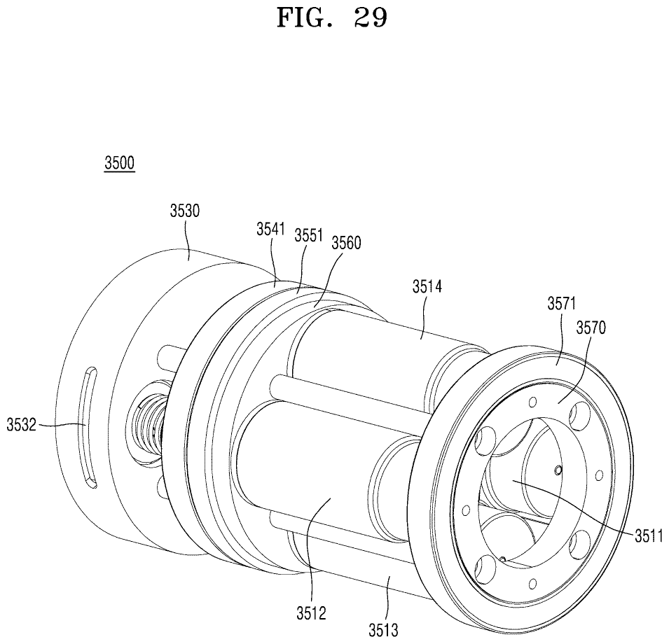

FIG. 27 is a diagram showing a manipulation portion 3200 and a power generation portion 3500 according to the other embodiment of the present disclosure, FIG. 28 is a perspective view of the power generation portion 3500 of FIG. 27, and FIG. 29 is a rear view of the power generation portion 3500 of FIG. 28.

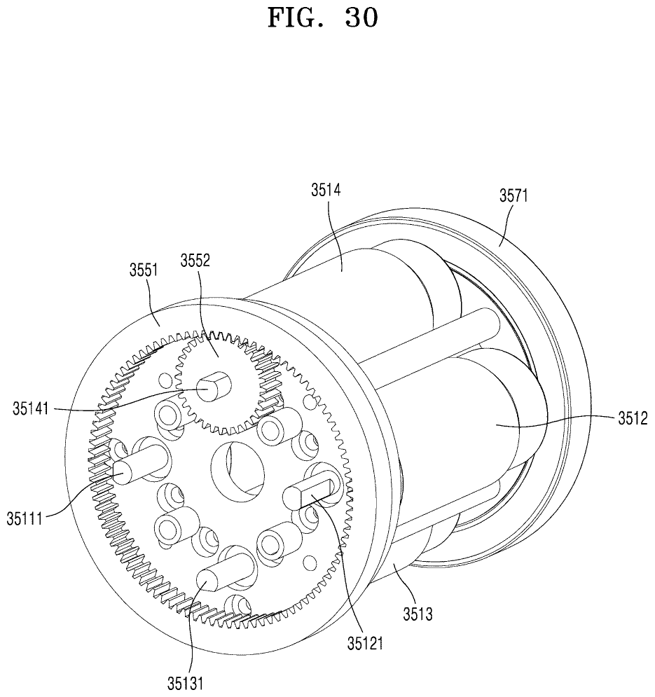

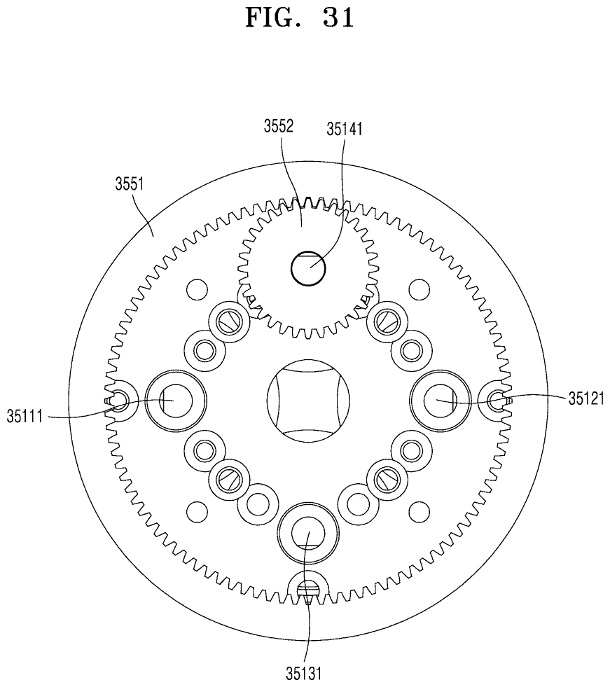

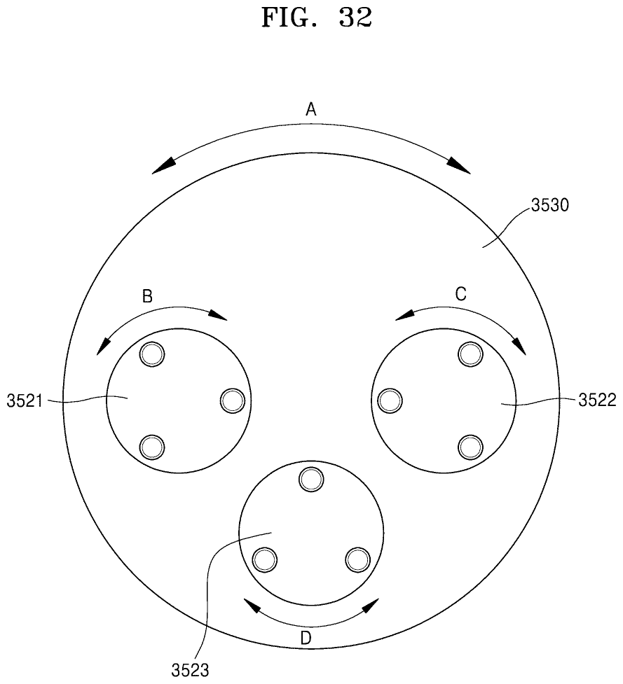

FIG. 30 is a diagram for describing a gear structure of the power generation portion 3500 of FIG. 28, FIG. 31 is a front view of the structure of FIG. 30, and FIG. 32 is a diagram for describing the rotation of the power generation portion 3500 of FIG. 28. Referring to FIGS. 27 to 32, a surgical instrument according to an embodiment of the present disclosure may include a power generation portion 3500 that generates power to control the end tool 3100.

The power generation portion 3500 may be disposed to be at least partially accommodated in a housing 3201 of the manipulation portion 3200.

Here, the housing 3201 may refer to a component that forms the outer shape of the manipulation portion 3200, but, for a driving module for a surgical instrument, may refer to a component that forms the outer shape of a module body. When a user manipulates the manipulation portion 3200, the power generation portion 3500 may generate power to control the end tool 3100 based on the manipulation of the user.

The power generation portion 3500 may include a motor pack 3510 including at least one motor.

The motor pack 3510 may roll around the axis in the direction in which the connection portion 3400 extends.

Here, the roll movement used in the present disclosure is defined as follows.

The roll movement refers to a movement that the end tool 3100, connection portion 3400, motor pack 3510, etc., which constitute the surgical instrument 3000, rotate around the axis in the direction in which the connection portion 3400 extends.

In other words, the roll movement refers to a rotation without bending in the Y-axis direction of FIG. 3 or the Z-axis direction of FIG. 3 around the direction (X-axis direction of FIG. 3) in which the connection portion 3400 extends. Referring back to FIG. 8, at least a portion of the power generation portion 3500 may be accommodated inside the housing 3201 of the manipulation portion 3200.

In this case, the motor pack 3510 is accommodated inside the housing 3201 of the manipulation portion 3200. Here, the rolling of the motor pack 3510 may mean rotation of the motor pack 3510 along the inner circumferential surface of the housing 3201 inside the housing 3201. In other words, when the user holds a handle 3202 of the manipulation portion 3200 and performs a manipulation to roll the motor pack 3510, the motor pack 3510 may rotate around a direction in which the connection portion 3400 extends inside the housing 3201 while the housing 3201 and the handle 3202 of the manipulation portion 3200 are fixed in place. In other words, it may be said that when a user performs a manipulation for a roll motion while holding the connection portion 3400, the housing 3201 and the handle 3202 rotate. The motor pack 3510 may include at least one motor.

For example, the motor pack 3510 may include at least one motor that generates power to drive the end tool 3100 based on a signal input to the manipulation portion 3200. According to an optional embodiment, the motor pack 3510 may further include a roll drive motor 3514. The motor pack 3510 may include a yaw drive motor 3511.

The yaw drive motor 3511 may generate power to yaw-rotate the end tool 3100. For example, the yaw drive motor 3511 may generate driving force for yaw-rotating the end tool 3100 when the user manipulates the manipulation portion 3200 to yaw-rotate the end tool 3100. The driving force generated by the yaw drive motor 3511 may be transmitted to the power transmission portion 3300 and rotate the yaw pulley 3320, and, as the first yaw wire 3361 and the second yaw wire 3362 are moved by the rotation of the yaw pulley 3320, the end tool 3100 may yaw-rotate.

The yaw drive motor 3511 may include a yaw motor rotation shaft 35111 extending in one direction.

The yaw motor rotation shaft 35111 is a part that rotates when the yaw drive motor 3511 starts driving. For example, the yaw motor rotation shaft 35111 may be formed to extend from the main body of the yaw drive motor 3511 in a direction toward the power transmission portion 3300. A yaw motor plate 3521 may be disposed at one end of the yaw motor rotation shaft 35111, as will be described later, and, when the yaw motor rotation shaft 35111 rotates, the yaw motor plate 3521 may rotate together. The yaw motor plate 3521 may be connected to the yaw pulley 3320, and driving force may be transmitted to the yaw pulley 3320 as the yaw motor plate 3521 rotates. The motor pack 3510 may include a pitch drive motor 3512.

The pitch drive motor 3512 may generate power to pitch-rotate the end tool 3100. For example, the pitch drive motor 3512 may generate driving force for pitch-rotating the end tool 3100 when the user manipulates the manipulation portion 3200 to pitch-rotate the end tool 3100. The driving force generated by the pitch drive motor 3512 may be transmitted to the power transmission portion 3300 and rotate the pitch pulley 3330, and, as the first pitch wire 3363 and the second pitch wire 3364 are moved by the rotation of the pitch pulley 3330, the end tool 3100 may pitch-rotate.

The pitch drive motor 3512 may include a pitch motor rotation shaft 35121 extending in one direction.

The pitch motor rotation shaft 35121 is a part that rotates when the pitch drive motor 3512 starts driving. For example, the pitch motor rotation shaft 35121 may be formed to extend from the main body of the pitch drive motor 3512 in a direction toward the power transmission portion 3300. A pitch motor plate 3522 may be disposed at one end of the pitch motor rotation shaft 35121, as will be described later, and, when the pitch motor rotation shaft 35121 rotates, the pitch motor plate 3522 may rotate together. The pitch motor plate 3522 may be connected to the pitch pulley 3330, and driving force may be transmitted to the pitch pulley 3330 as the pitch motor plate 3522 rotates. The power generation portion 3500 may include the roll drive motor 3514 that generates power to roll-rotate the motor pack 3510.

For example, the roll drive motor 3514 may generate driving force for roll-rotating the motor pack 3510 when a user manipulates the manipulation portion 3200 to rotate the motor pack 3510. According to an embodiment, the roll drive motor 3514 may be provided in the motor pack 3510.

For example, the roll drive motor 3514 may be provided inside the motor pack 3510 together with the yaw drive motor 3511 and the pitch drive motor 3512. The roll drive motor 3514 may be driven by manipulation of a user to generate driving force to rotate the motor pack 3510.

In this case, the roll drive motor 3514 may move together with the motor pack 3510. For example, when the motor pack 3510 is roll-rotated by the roll drive motor 3514, the roll drive motor 3514 may rotate together with the motor pack 3510 as a component included in the motor pack 3510. According to another embodiment, the roll drive motor 3514 may not be provided in the motor pack 3510.

For example, the roll drive motor 3514 may be disposed outside the motor pack 3510. The roll drive motor 3514 may be driven by manipulation of a user to generate driving force to rotate the motor pack 3510.

In this case, the roll drive motor 3514 may move independently from the motor pack 3510. For example, unlike as described later, the motor pack 3510 may be roll-rotated by the roll drive motor 3514, but the roll drive motor 3514 may not rotate together with the motor pack 3510. In detail, the yaw drive motor 3511, the pitch drive motor 3512, and a firing drive motor 3513 may rotate together, but the roll drive motor 3514 may not rotate together with the yaw drive motor 3511, the pitch drive motor 3512, and the firing drive motor 3513. Hereinafter, for convenience of explanation, descriptions will be given under the assumption that the roll drive motor 3514 is provided in the motor pack 3510. However, one of ordinary skill in the art will understand that the roll drive motor 3514 may not be provided in the motor pack 3510 in the descriptions below.

Also, one of ordinary skill in the art will understand that, when the roll drive motor 3514 is not provided in the motor pack 3510, descriptions given below may be appropriately modified and applied. The motor pack 3510 may include the firing drive motor 3513.

The firing drive motor 3513 may generate power to linearly move the working member 3154 of the end tool 3100. For example, when a user manipulates the manipulation portion 3200 to linearly move the working member 3154 of the end tool 3100, the firing drive motor 3513 may generate driving force to linearly move the working member 3154. The firing drive motor 3513 may include a firing motor rotation shaft 35131 extending in one direction.

The firing motor rotation shaft 35131 is a part that rotates when the firing drive motor 3513 starts driving. For example, the firing motor rotation shaft 35131 may be formed to extend from the main body of the firing drive motor 3513 in a direction toward the power transmission portion 3300. A firing motor plate 35231 may be disposed at one end of the firing motor rotation shaft 35131, as will be described later, and, when the firing motor rotation shaft 35131 rotates, the firing motor plate 35231 may rotate together. The firing motor plate 35231 may be connected to the firing pulley 3340, and, as the firing motor plate 35231 rotates, driving force may be transmitted to the firing pulley 3340. The motor pack 3510 may include a base plate 3560.

The base plate 3560 may be disposed in front of the yaw drive motor 3511, the pitch drive motor 3512, the roll drive motor 3514, and the firing drive motor 3513. The base plate 3560 may be connected to the roll drive motor 3514, the yaw drive motor 3511, the pitch drive motor 3512, and the firing drive motor 3513. In other words, it may be said that the roll drive motor 3514, the yaw drive motor 3511, the pitch drive motor 3512, and the firing drive motor 3513 are connected to the base plate 3560. In other words, it may be said that the base plate 3560 connects the roll drive motor 3514, the yaw drive motor 3511, the pitch drive motor 3512, and the firing drive motor 3513 to one another, such that the roll drive motor 3514, the yaw drive motor 3511, the pitch drive motor 3512, and the firing drive motor 3513 move or rotate together. Therefore, when the base plate 3560 rotates due to the driving force of the roll drive motor 3514, the roll drive motor 3514, the yaw drive motor 3511, the pitch drive motor 3512, and the firing drive motor 3513 connected to the base plate 3560 may simultaneously rotate.

In other words, when the roll drive motor 3514 is driven, the base plate 3560 is rotated thereby, and, when the base plate 3560 rotates, the roll drive motor 3514, the yaw drive motor 3511, the pitch drive motor 3512, and the firing drive motor 3513 connected to the base plate 3560 rotate together with the base plate 3560. Here, since the base plate 3560 rotates around the axis in the direction in which the connection portion 3400 extends, the motor pack 3510 including the base plate 3560, the roll drive motor 3514, the yaw drive motor 3511, the pitch drive motor 3512, and the firing drive motor 3513 roll-rotates around the axis in the direction in which the connection portion 3400 extends. According to an embodiment, the roll drive motor 3514, the yaw drive motor 3511, the pitch drive motor 3512, and the firing drive motor 3513 may be arranged in parallel with one another.

Also, the roll drive motor 3514, the yaw drive motor 3511, the pitch drive motor 3512, the firing drive motor 3513, and the firing drive motor 3513 may be arranged to form a circular pattern. As described above, the motor pack 3510 may roll-rotate inside the housing 3201 of the manipulation portion 3200.

In this case, since the motor pack 3510 includes a plurality of motors, the roll drive motor 3514, the yaw drive motor 3511, the pitch drive motor 3512, and the firing drive motor 3513 may be arranged to form a circular pattern with one another, thereby minimizing the space needed by the motor pack 3510 to rotate. In other words, as the roll drive motor 3514, the yaw drive motor 3511, the pitch drive motor 3512, and the firing drive motor 3513 are arranged to form a circular pattern, the housing 3201 may be designed to have a small inner diameter needed for rotation of the motor pack 3510, thereby contributing miniaturization and weight reduction of the surgical instrument 3000. Meanwhile, here, arranging the roll drive motor 3514, the yaw drive motor 3511, the pitch drive motor 3512, and the firing drive motor 3513 to form a circular pattern does not mean that they are arranged at equal intervals between one another, and any arrangement in which the outer circumferential surfaces of the roll drive motor 3514, the yaw drive motor 3511, the pitch drive motor 3512, and the firing drive motor 3513 are arranged within one circle will suffice.

According to the other embodiment, the roll drive motor 3514, the yaw drive motor 3511, the pitch drive motor 3512, and the firing drive motor 3513 may be provided to have different performances.

For example, amounts of driving force that need to be generated by the roll drive motor 3514, the yaw drive motor 3511, the pitch drive motor 3512, and the firing drive motor 3513 to perform their respective roles may be different from one another. To this end, the roll drive motor 3514, the yaw drive motor 3511, the pitch drive motor 3512, and the firing drive motor 3513 may have different outputs or different sizes as needed. At least one through hole may be formed in the base plate 3560.

For example, at least as many through holes as the number of motors included in the motor pack 3510 may be formed in the base plate 3560. The through hole is a hole through which the rotation shaft of each motor passes. For example, the yaw motor rotation shaft 35111 extends from the main body of the yaw drive motor 3511 and may be formed to extend through the base plate 3560 (through a through hole).

Also, the pitch motor rotation shaft 35121 extends from the main body of the pitch drive motor 3512 and may be formed to extend through the base plate 3560 (through a through hole). Also, the firing motor rotation shaft 35131 extends from the main body of the firing drive motor 3513 and may be formed to extend through the base plate 3560 (through a through hole). Meanwhile, the roll drive motor 3514 may include a roll motor rotation shaft 35141 extending in one direction.

The roll motor rotation shaft 35141 is a part that rotates when the roll drive motor 3514 starts driving. For example, the roll motor rotation shaft 35141 may be formed to extend in a forward direction from the main body of the roll drive motor 3514. In other words, the roll motor rotation shaft 35141 extends forward from the main body of the roll drive motor 3514 and may be formed to extend through the base plate 3560. Hereinafter, the principle of rotation of the motor pack 3510 will be described in detail.

Referring to FIGS. 11 and 12, the power generation portion 3500 may include a first gear 3551 formed in a circular shape and a second gear 3552 engaged with the first gear 3551.

The first gear 3551 is formed in the shape of a hollow circle, and gear teeth may be formed on the inner circumferential surface of the hollow circle.

In other words, the first gear 3551 may be a ring gear with gear teeth formed on the inner circumferential surface thereof. The second gear 3552 is a gear with gear teeth formed on the outer circumferential surface thereof and may be engaged with the first gear 3551.

The second gear 3552 may be disposed on the roll motor rotation shaft 35141. In other words, the roll motor rotation shaft 35141 is formed to extend forward from the main body of the roll drive motor 3514 to penetrate through the base plate 3560, and the second gear 3552 may be disposed at an extended portion of the roll motor rotation shaft 35141. At this time, the second gear 3552 is coupled to the roll motor rotation shaft 35141 and, when the roll motor rotation shaft 35141 rotates, the second gear 3552 may rotate together. The first gear 3551 may be disposed in front of the base plate 3560.

Also, the first gear 3551 may be fixed to the inner circumferential surface of the housing 3201. Therefore, when the roll drive motor 3514 is driven, the motor pack 3510 may roll-rotate within the housing 3201. In detail, when the roll drive motor 3514 is driven, the roll motor rotation shaft 35141 may rotate, and the second gear 3552 disposed on the roll motor rotation shaft 35141 may rotate together.

At this time, when the second gear 3552 rotates, the first gear 3551 engaged with the second gear 3552 is fixed to the inner circumferential surface of the housing 3201, and thus the second gear 3552 moves along the gear teeth of the first gear 3551. In other words, when the roll drive motor 3514 starts driving, the first gear 3551 and the second gear 3552 rotate relatively. At this time, since the first gear 3551 is fixed to the housing 3201, the second gear 3552 relatively moves along the first gear 3551. Also, the second gear 3552 is connected to the roll motor rotation shaft 35141, the roll drive motor 3514 is connected to the base plate 3560, and the base plate 3560 is connected to the yaw drive motor 3511 and the pitch drive motor 3512. Therefore, the motor pack 3510 may rotate relative to the housing 3201 due to the movements of the first gear 3551 and the second gear 3552. In other words, the motor pack 3510 may rotate independently of the movement of the housing 3201. To explain this in more detail, the base plate 3560 may rotate relative to the housing 3201.

In other words, since the second gear 3552 is connected to the base plate 3560 by the roll motor rotation shaft 35141, when the second gear 3552 moves, as the second gear 3552 moves along the first gear 3551, the base plate 3560 rotates relative to the housing 3201. Here, since the roll motor rotation shaft 35141 is eccentric with respect to the rotation shaft of the base plate 3560, when the second gear 3552 moves along the first gear 3551, the base plate 3560 may rotate relative to the housing 3201 instead of changing its position to follow the second gear 3552. Meanwhile, as will be described later, a bearing plate 3540 may also be connected to the second gear 3552 through the roll motor rotation shaft 35141.

Therefore, when the second gear 3552 rotates, the bearing plate 3540 may rotate relative to the housing 3201 as the second gear 3552 moves along the first gear 3551. In this case, as will be described later, a first bearing 3541 to reduce rotational friction of the bearing plate 3540 may be disposed coaxially with the bearing plate 3540 and in contact with the inner circumferential surface of the housing 3201. Therefore, the bearing plate 3540 may easily rotate. Meanwhile, although gear teeth of the first gear 3551 and the second gear 3552 are shown in the shape of spur gears in the drawings, the present disclosure is not limited thereto, and the first gear 3551 and the second gear 3552 may have various shapes such as helical gears and herringbone gears.

Meanwhile, when the roll drive motor 3514 is disposed outside the motor pack 3510, the first gear 3551 and the second gear 3552 may be formed at different positions.

According to an embodiment, the power generation portion 3500 may further include the bearing plate 3540 and a first bearing 3541.

The bearing plate 3540 and the first bearing 3541 may reduce rotational friction between the motor pack 3510 and the housing 3201 when the motor pack 3510 roll-rotates. The bearing plate 3540 may be disposed in front of the first gear 3551.

At least one through hole may be formed in the bearing plate 3540.

The through hole is a hole through which the rotation shafts of respective motors pass. For example, the yaw motor rotation shaft 35111 may be formed to extend from the main body of the yaw drive motor 3511 to penetrate through the base plate 3560 and the bearing plate 3540.

Also, the pitch motor rotation shaft 35121 may be formed to extend from the main body of the pitch drive motor 3512 to penetrate through the base plate 3560 and the bearing plate 3540. Also, the firing motor rotation shaft 35131 may be formed to extend from the main body of the firing drive motor 3513 to penetrate through the base plate 3560 and the bearing plate 3540. In this case, the roll motor rotation shaft 35141 extends from the main body of the roll drive motor 3514 to penetrate through the base plate 3560, but may not extend to the bearing plate 3540. In this regard, since the yaw motor rotation shaft 35111, the pitch motor rotation shaft 35121, and the firing motor rotation shaft 35131 are formed to extend to penetrate through the bearing plate 3540, when the motor pack 3510 rotates, the base plate 3560 and the bearing plate 3540 may rotate together.

The first bearing 3541 may be disposed on an outer circumferential surface of the bearing plate 3540.

For example, the first bearing 3541 may be disposed to cover the outer circumferential surface of the bearing plate 3540. Therefore, when the motor pack 3510 roll-rotates, the bearing plate 3540 rotates together with the motor pack 3510, and, at this time, the bearing plate 3540 and the first bearing 3541 may reduce rotational friction between the motor pack 3510 and the housing 3201. According to the other embodiment, the power generation portion 3500 may further include a circuit plate 3570 and a second bearing 3571.

The circuit plate 3570 and the second bearing 3571 may reduce rotational friction between the motor pack 3510 and the housing 3201 when the motor pack 3510 roll-rotates. The circuit plate 3570 may be disposed behind the motor pack 3510.

The circuit plate 3570 is a part to which a circuit unit 3600 are connected, as will be described later.

The circuit plate 3570 is connected to the motor pack 3510 and may rotate together when the motor pack 3510 rotates.

The second bearing 3571 may be disposed on the outer circumferential surface of the circuit plate 3570.

Therefore, when the motor pack 3510 roll-rotates, the circuit plate 3570 rotates together with the motor pack 3510, and, at this time, the circuit plate 3570 and the second bearing 3571 may reduce rotational friction between the motor pack 3510 and the housing 3201. The power generation portion 3500 may further include a pulley coupling plate 3530.

The pulley coupling plate 3530 is a part to which the power transmission portion 3300 is connected.

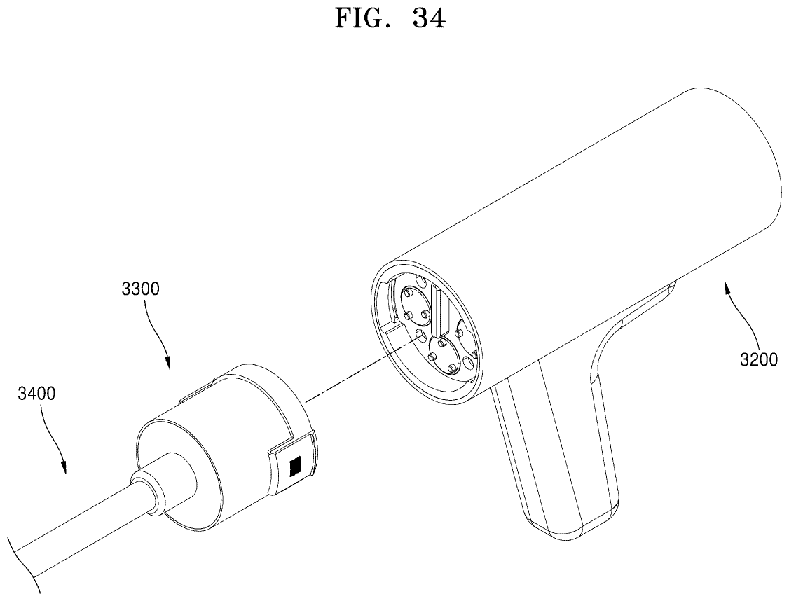

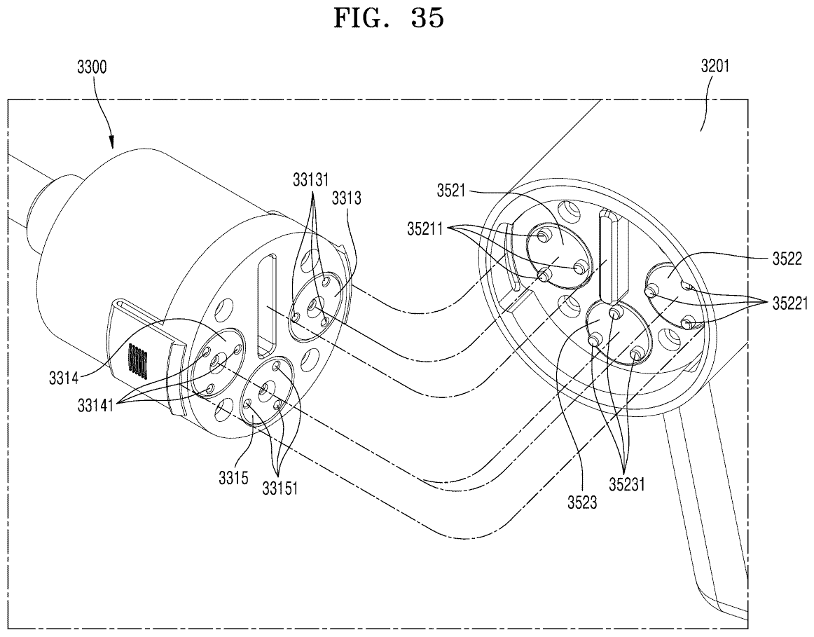

FIGS. 34 and 35 are diagrams for describing the coupling structure of a surgical instrument according to an embodiment of the present disclosure.

Referring to FIGS. 34 and 35 together, according to an embodiment, the power transmission portion 3300 may be detachably coupled to the power generation portion 3500. For example, the power transmission portion 3300 may be detachably coupled to the pulley coupling plate 3530. Therefore, after a user uses components (the power transmission portion 3300, the connection portion 3400, and the end tool 3100) in the direction from the power transmission portion 3300 toward the distal end, the user may discard the components, couple a new product to the manipulation portion 3200 accommodating the power generation portion 3500, and use the surgical instrument 3000 again. According to the other embodiment, at least one coupling member 3316 may be formed at the pulley frame 3310 of the power transmission portion 3300.

A hook 33161 may be formed at the coupling member 3316. Also, the pulley coupling plate 3530 may include an internal space for accommodating at least a portion of the pulley frame 3310 and a wall formed along the circumference of the pulley coupling plate 3530 to define the internal space. At this time, a hook groove 3532 in which the hook 33161 is inserted and fixed may be formed on the wall. Therefore, when the pulley frame 3310 is inserted into the inner space of the pulley coupling plate 3530, the hook 33161 may be inserted into the hook groove 3532 and fixed. According to the other embodiment, the pulley coupling plate 3530 may include a protruding coupling block 3531, and the pulley frame 3310 may include an insertion groove into which the protruding coupling block 3531 is inserted.

Therefore, when the pulley coupling plate 3530 and the pulley frame 3310 are coupled, the protruding coupling block 3531 is inserted into the insertion groove, and thus the pulley coupling plate 3530 and the pulley frame 3310 may be coupled to each other at a predetermined position. Also, since the protruding coupling block 3531 is inserted into the insertion groove, the roll rotational force of the pulley coupling plate 3530 may be transmitted to the pulley frame 3310 through the protruding coupling block 3531 and the insertion groove. According to an optional embodiment, the protruding coupling block 3531 may be formed in the shape of a long bar, and, in this case, the insertion groove may be formed in a shape corresponding to the shape of the protruding coupling block 3531. According to the other embodiment, although not shown in the drawings, the surgical instrument 3000 according to the present disclosure may further include a waterproof structure.

According to a specific example, at least one O-ring may be provided inside the housing 3201.

For example, an O-ring may be provided between the outer circumferential surface of the pulley coupling plate 3530 and the housing 3201. The O-ring is disposed on the outer circumferential surface of the pulley coupling plate 3530 to be in close contact with the housing 3201 to prevent water, etc. from penetrating between the power generation portion 3500 and the housing 3201. In another example, an O-ring may be provided at least at one location between the yaw motor plate 3521 and the pulley coupling plate 3530 and between the pitch motor plate 3522 and the pulley coupling plate 3530.

An O-ring may be disposed to fit tightly between yaw motor plate 3521 or the pitch motor plate 3522 and the pulley coupling plate 3530 to prevent water, etc. from penetrating between the yaw motor plate 3521, the pitch motor plate 3522 and the pulley coupling plate 3530. According to the other embodiment, the pulley coupling plate 3530 and the bearing plate 3540 may be coupled to each other by at least one bolt.

In this case, at least one seal washer may be disposed under the bolt in a bolt hole into which the bolt is inserted. The seal washer may prevent water, etc. from penetrating through the bolt hole. At least one through hole may be formed in the pulley coupling plate 3530.

For example, the two through holes may be formed in the pulley coupling plate 3530. The yaw motor plate 3521, the pitch motor plate 3522, and the firing motor plate 35231 may be arranged in the through holes formed in the pulley coupling plate 3530.

The yaw motor plate 3521 may be rotated by driving force generated by the yaw drive motor 3511.

The yaw motor plate 3521 may be disposed at one end of the yaw motor rotation shaft 35111. For example, when the yaw motor rotation shaft 35111 rotates, the yaw motor plate 3521 may rotate together. In other words, it may be said that the yaw motor plate 3521 is a member that transmits driving force generated by the yaw drive motor 3511 to the power transmission portion 3300. At least one first protrusion 35211 may be formed on the yaw motor plate 3521.

The at least one first protrusion 35211 is a portion that protrudes outward from the yaw motor plate 3521. The at least one first protrusion 35211 may be inserted into at least one first insertion hole 33131 formed in a yaw pulley plate 3313, as described later. The pitch motor plate 3522 may be rotated by driving force generated by the pitch drive motor 3512.

The pitch motor plate 3522 may be disposed at one end of the pitch motor rotation shaft 35121. For example, when the pitch motor rotation shaft 35121 rotates, the pitch motor plate 3522 may rotate together. In other words, it may be said that the pitch motor plate 3522 is a member that transmits driving force generated by the pitch drive motor 3512 to the power transmission portion 3300. At least one second protrusion 35221 may be formed on the pitch motor plate 3522.

The at least one second protrusion 35221 is a portion that protrudes outward from the pitch motor plate 3522. The at least one second protrusion 35221 may be inserted into at least one second insertion hole 33141 formed in a pitch pulley plate 3314, as described later. The firing motor plate 35231 may be rotated by driving force generated by the firing drive motor 3513.

The firing motor plate 35231 may be disposed at one end of the firing motor rotation shaft 35131. For example, when the firing motor rotation shaft 35131 rotates, the firing motor plate 35231 may rotate together. In other words, it may be said that the firing motor plate 35231 is a member that transmits driving force generated by the firing drive motor 3513 to the power transmission portion 3300. At least one third protrusion 3523 may be formed on the firing motor plate 35231.

The at least one third protrusion 3523 is a portion that protrudes outward from the firing motor plate 35231. The at least one third protrusion 3523 may be inserted into at least one third insertion hole 33151 formed in a firing pulley plate 3315, as described later. The yaw pulley plate 3313, the pitch pulley plate 3314, and the firing pulley plate 3315 may be arranged at the pulley frame 3310.

The yaw pulley plate 3313 may be formed to be rotatable.

In detail, the yaw pulley plate 3313 may be coupled to the yaw motor plate 3521 and may rotate together when the yaw motor plate 3521 rotates. The yaw pulley plate 3313 is a part connected to the yaw pulley 3320, and, when the yaw pulley plate 3313 rotates, the yaw pulley 3320 may rotate together. In other words, when power transmitted from the outside rotates the yaw pulley plate 3313, the yaw pulley 3320 may rotate together. In other words, it may be said that the yaw pulley plate 3313 is a part that receives driving force generated by the yaw drive motor 3511 and transmits the driving force to the yaw pulley 3320. The yaw pulley plate 3313 may include the at least one first insertion hole 33131.

The at least one first insertion hole 33131 is a part into which the at least one first protrusion 35211 of the yaw motor plate 3521 is inserted. In this way, the yaw motor plate 3521 and the yaw pulley plate 3313 may be stably coupled to each other through coupling between the at least one first protrusion 35211 and the at least one first insertion hole 33131, and driving force of the yaw drive motor 3511 may be efficiently transmitted to the yaw pulley 3320. The pitch pulley plate 3314 may be formed to be rotatable.

In detail, the pitch pulley plate 3314 may be coupled to the pitch motor plate 3522 and may rotate together when the pitch motor plate 3522 rotates. The pitch pulley plate 3314 is a part connected to the pitch pulley 3330, and, when the pitch pulley plate 3314 rotates, the pitch pulley 3330 may rotate together. In other words, when power transmitted from the outside rotates the pitch pulley plate 3314, the pitch pulley 3330 may rotate together. In other words, it may be said that the pitch pulley plate 3314 is a part that receives driving force generated by the pitch drive motor 3512 and transmits the driving force to the pitch pulley 3330. The pitch pulley plate 3314 may include the at least one second insertion hole 33141.

The at least one second insertion hole 33141 is a part into which the at least one second protrusion 35221 of the pitch motor plate 3522 is inserted. In this way, the pitch motor plate 3522 and the pitch pulley plate 3314 may be stably coupled to each other through coupling between the at least one second protrusion 35221 and the at least one second insertion hole 33141, and driving force of the pitch drive motor 3512 may be efficiently transmitted to the pitch pulley 3330. The firing pulley plate 3315 may be formed to be rotatable.

In detail, the firing pulley plate 3315 may be coupled to the firing motor plate 35231 and may rotate together when the firing motor plate 35231 rotates. The firing pulley plate 3315 is a part connected to the firing pulley 3340, and, when the firing pulley plate 3315 rotates, the firing pulley 3340 may rotate together. In other words, when power transmitted from the outside rotates the firing pulley plate 3315, the firing pulley plate 3315 may rotate together. In other words, it may be said that the firing pulley plate 3315 is a part that receives driving force generated by the firing drive motor 3513 and transmits the driving force to the firing pulley 3340. The firing pulley plate 3315 may include at least one third insertion hole 33151.

The at least one third insertion hole 33151 is a part into which the at least one third protrusion 3523 of the firing motor plate 35231 is inserted. In this way, the firing motor plate 35231 and the firing pulley plate 3315 may be stably coupled to each other through coupling between the at least one third protrusion 3523 and the at least one third insertion hole 33151, and driving force of the firing drive motor 3513 may be efficiently transmitted to the firing pulley 3340. According to the other embodiment, the yaw drive motor 3511, the pitch drive motor 3512, the roll drive motor 3514, and the firing drive motor 3513 may be driven independently of one another.

Therefore, the yaw drive motor 3511, the pitch drive motor 3512, the roll drive motor 3514, and the firing drive motor 3513 may perform the yaw rotation of the end tool 3100, the pitch rotation of the end tool 3100, the roll rotation of motor pack 3510, and the linear movement of the working member 3154 independently of one another. Referring back to FIG. 32, the pulley coupling plate 3530 may rotate in a first direction A as the roll drive motor 3514 is driven.

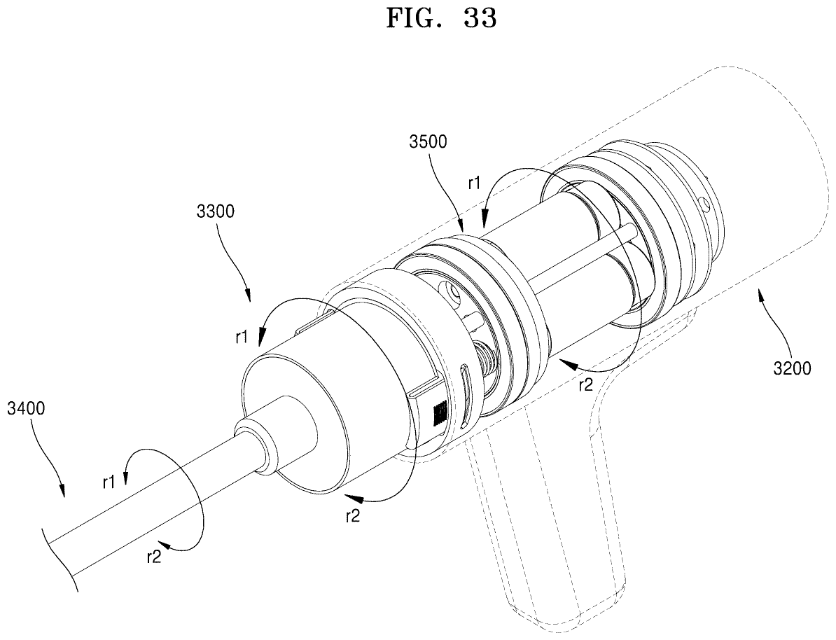

At this time, since the yaw drive motor 3511 may be driven independently, the yaw drive motor 3511 may be driven independently regardless of the driving of the roll drive motor 3514 and rotate the yaw motor plate 3521 in a second direction B. Also, since the pitch drive motor 3512 may be driven independently, the pitch drive motor 3512 may be driven independently regardless of the driving of the roll drive motor 3514 and the yaw drive motor 3511 and rotate the pitch motor plate 3522 in a third direction C. Also, since the firing drive motor 3513 may be driven independently, the firing drive motor 3513 may be driven independently regardless of the driving of the roll drive motor 3514, the yaw drive motor 3511, and the pitch drive motor 3512 and rotate the firing motor plate 35231 in a fourth direction D. In other words, the end tool 3100 may perform only one of pitch rotation, yaw rotation, roll rotation, and linear movement of the working member 3154 or may perform multiple motions simultaneously. FIG. 33 is a diagram for describing the roll operation of a surgical instrument according to the other embodiment of the present disclosure.

Referring to FIG. 33, a surgical instrument according to the other embodiment of the present disclosure may be formed such that the motor pack 3510 may roll-rotate.

Here, a power generation portion 3500 including the motor pack 3510 may include the pulley frame 3310 coupled to the power transmission portion 3300 at the forefront.

The pulley frame 3310 may be coupled to the power transmission portion 3300. Therefore, the power transmission portion 3300 may roll-rotate together with the pulley coupling plate 3530 while being coupled to the pulley coupling plate 3530. Also, the connection portion 3400 may be connected to the power transmission portion 3300.

Therefore, the connection portion 3400 may roll-rotate together with the roll-rotation of the power transmission portion 3300. Also, the end tool 1100 disposed on one side of the connection portion 3400 may be connected to the connection portion 3400.

Therefore, the end tool 1100 may roll-rotate together with the rotation of the connection portion 3400. Ultimately, according to the present disclosure, when the roll drive motor 3514 is driven by a user manipulating the manipulation portion 3200, the components excluding the manipulation portion 3200 may roll-rotate around the extending lengthwise direction of the connection portion 3400.

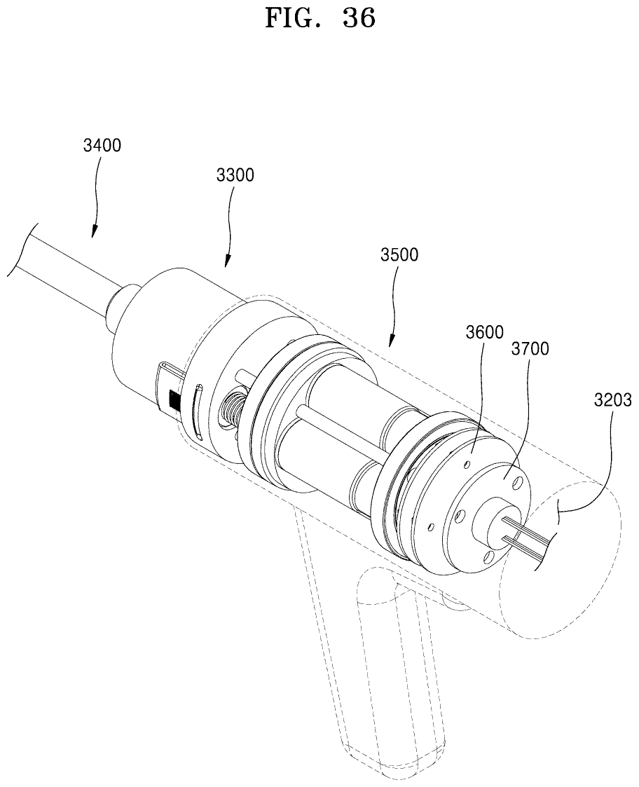

FIG. 36 is a diagram showing the internal structure of a surgical instrument according to the other embodiment of the present disclosure.

Referring to FIG. 36, a manipulation portion internal space 3203 may be provided inside the manipulation portion 3200.

The circuit unit 3600 may be disposed in the manipulation portion internal space 3203.

The circuit unit 3600 includes an electronic circuit for controlling the operation of the motor pack 3510.

According to the other embodiment, the circuit unit 3600 may include a motor driver, a motor controller, and a micro controller unit. However, the present disclosure is not limited thereto, and anything for driving the motor pack 3510 may be used. The circuit unit 3600 may be disposed on one side of the motor pack 3510.

For example, in FIG. 36, the circuit unit 3600 may be disposed behind the motor pack 3510 (i.e., in a direction opposite to the connection portion 3400). In detail, the circuit unit 3600 may be connected to the circuit plate 3570 of the power generation portion 3500. Therefore, when the motor pack 3510 roll-rotates, the circuit unit 3600 may rotate together with the motor pack 3510. Although not shown in the drawing, the circuit unit 3600 and the motor pack 3510 may be connected to each other through a plurality of wires to drive the motor pack 3510.

Therefore, as the motor pack 3510 roll-rotates, the circuit unit 3600 also rotates together, thereby preventing a plurality of wires connecting the motor pack 3510 and the circuit unit 3600 from being twisted. According to the other embodiment, a slip ring 3700 may be disposed on one side of the circuit unit 3600.

The slip ring 3700 is a component that connects the motor pack 3510 or the circuit unit 3600 that controls the operation of the motor pack 3510 to various electrical/electronic elements or connects communication. For example, the slip ring 3700 may electrically connect the motor pack 3510 or a circuit unit that controls the operation of the motor pack 3510 to a power source, a switch, a button, an OLED screen, and other circuit units. Here, the power source, the switch, the button, the OLED screen, and the other circuit units may be arranged inside the surgical instrument 3000 according to the present disclosure, or may be arranged outside the surgical instrument 3000. Also, the slip ring 3700 may connect communication between various elements for the operation of the surgical instrument 3000. For example, the slip ring 3700 may connect communication between at least some of the manipulation portion 3200, the power transmission portion 3300, the power generation portion 3500, and the circuit unit 3600. Here, the type of communication is not limited, and any type capable of connecting various elements of the surgical instrument 3000 through communication may be employed. As described above, the motor pack 3510 and the circuit unit 3600 are formed to be roll-rotatable.

Also, the motor pack 3510 and/or circuit unit 3600 may be electrically connected to various electrical/electronic elements. In this case, when the motor pack 3510 and/or the circuit unit 3600 are connected to electrical/electronic elements through wires, etc., a problem that the wires are twisted due to rotation of the motor pack 3510 and the circuit unit 3600 may occur. Therefore, as the slip ring 3700 is disposed on one side of the circuit unit 3600, even when the circuit unit 3600 rotates, the wires connecting the motor pack 3510 and/or the circuit unit 3600 to various electrical/electronic elements may not be twisted.

For example, the motor pack 3510 and the circuit unit 3600 may receive power stably from an external power source because the wires are not twisted. According to an embodiment, although not shown in the drawings, the surgical instrument 3000 may further include at least one sub-circuit unit.

The sub-circuit unit may be disposed inside the manipulation portion 3200. According to a preferred embodiment, the sub-circuit unit may be disposed at the handle (3202 of FIG. 27) of the manipulation portion 3200. In this case, the sub-circuit unit may not rotate even when the motor pack 3510 rotates. The sub-circuit unit may pre-process various signals for controlling the motor pack 3510.

Also, the sub-circuit unit may transmit pre-processed signals to the circuit unit 3600. To this end, the circuit unit 3600 and the sub-circuit unit may be connected through serial communication, etc. However, the present disclosure is not limited thereto, and the circuit unit 3600 and the sub-circuit unit may be connected to each other in various ways. Therefore, the number of wires that need to be connected to the circuit unit 3600 through the slip ring 3700 may be reduced. In a specific example, assuming that there are four buttons for manipulation on the manipulation portion 3200 and two wires (e.g., a ground wire and a communication wire) are needed to transmit and receive signals to and from each button, at least five wires need to be connected to circuit unit 3600.

In other words, even when a ground wire is used in common, at least one ground wire and four communication wires are needed to manipulate the manipulation portion 3200. In this case, when a sub-circuit unit is provided and signals of at least five wires are pre-processed once in the sub-circuit unit, the circuit unit 3600 and the four buttons of the manipulation portion 3200 may communicate with each other by using only two wires through communication connection function of the slip ring 3700. Therefore, through this configuration, the arrangement of wires may be simplified and the size of the slip ring 3700 may also be minimized. However, this example is merely one of several functions of the sub-circuit unit, and the sub-circuit unit may also pre-process signals from several wires. Therefore, technical idea of the present disclosure is not limited to the descriptions given above. According to the other embodiment, although not shown in the drawings, the surgical instrument may further include a component for setting the zero point of roll-rotation of the motor pack 3510, etc.

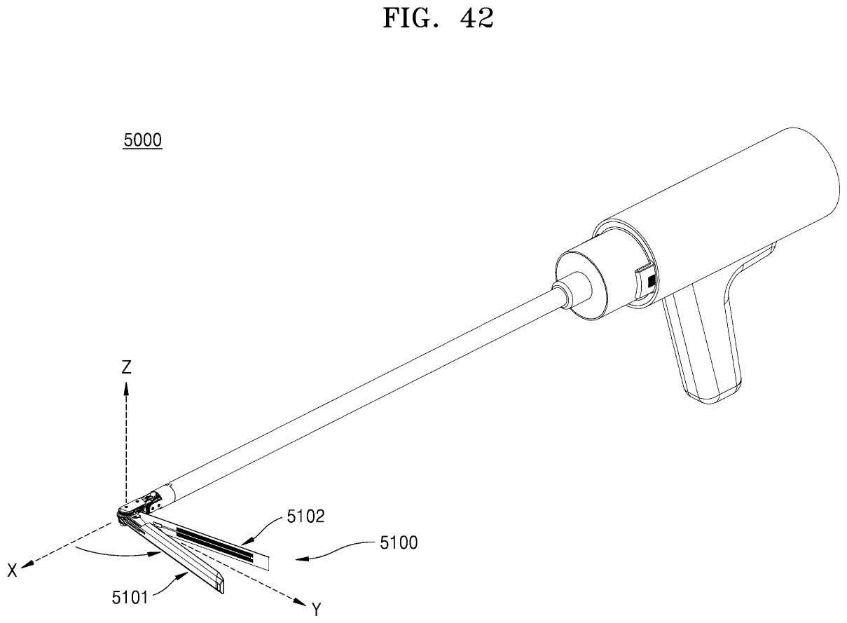

For example, the surgical instrument may further include at least one encoder for measuring the roll-rotation angle of the motor pack 3510, etc. Alternatively, the surgical instrument may further include a touch sensor, a Hall effect sensor, a photo sensor, etc. to measure the roll-rotation angle of the motor pack 3510, etc. However, the present disclosure is not limited thereto, and anything may be provided in the surgical instrument of the present disclosure as long as it is for measuring the roll-rotation angle of the motor pack 3510, etc. FIGS. 42 to 46 are diagrams showing the yaw rotation motion of a surgical instrument according to the other embodiment of the present disclosure.

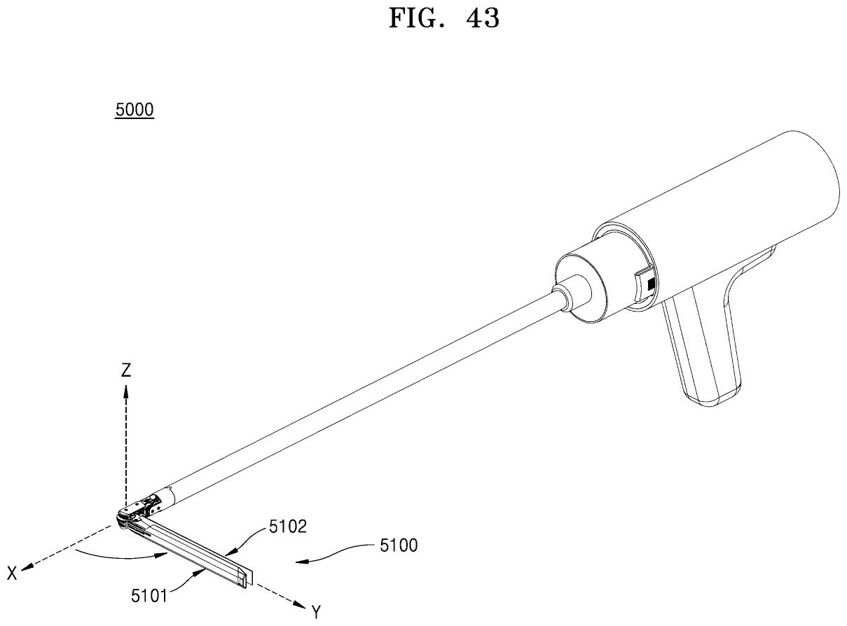

In detail, FIG. 42 is a diagram showing a state in which jaws are yaw-rotated by −90°, and FIG. 43 is a diagram showing a process of performing an actuation motion in the state in which jaws are yaw-rotated by −90°.

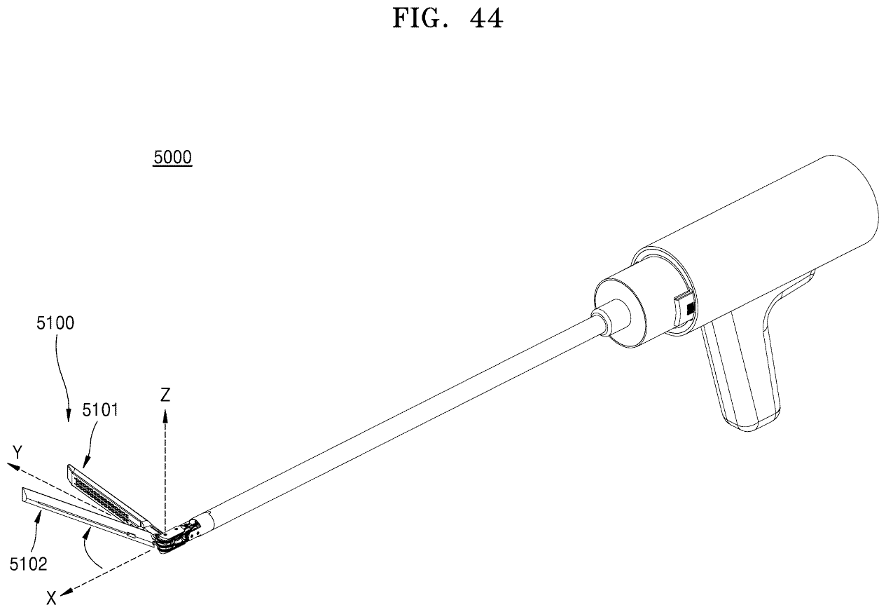

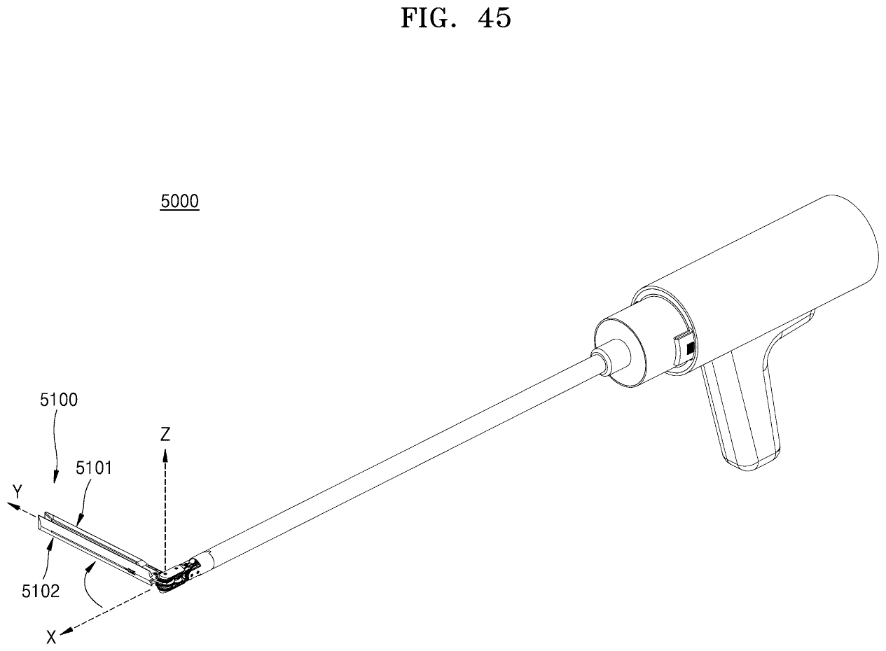

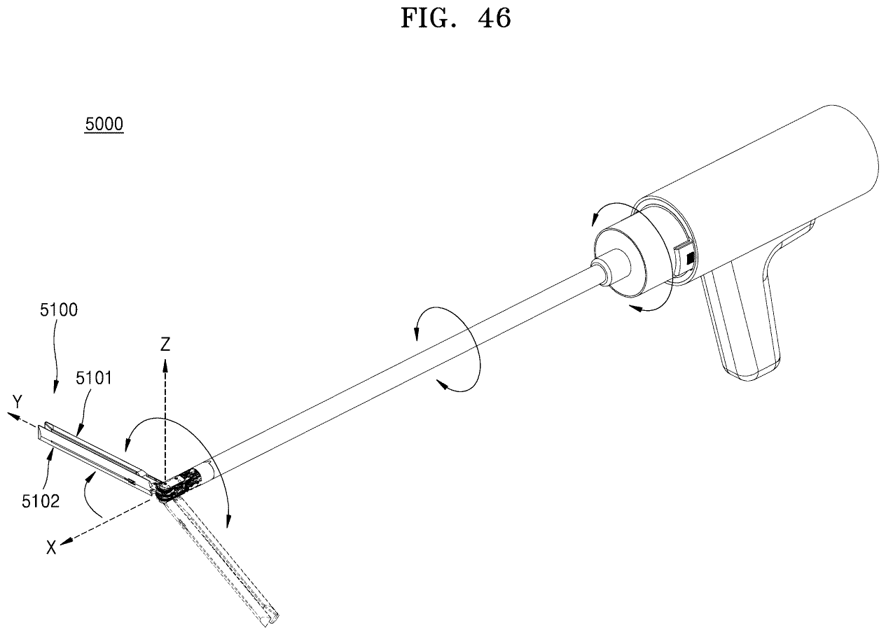

FIG. 44 is a diagram showing a state in which jaws are yaw-rotated by +90°, FIG. 45 is a diagram showing a process of performing an actuation motion in the state in which jaws are yaw-rotated by +90°, and FIG. 46 is a diagram showing a state in which a roll motion is performed while jaws are yaw-rotated. Referring to FIGS. 42 to 46, the surgical instrument 5000 according to the present disclosure may include the end tool 5100 including the first jaw 5101 and the second jaw 5102.

Here, the end tool 5100 of the surgical instrument 5000 may correspond to the end tool 1100 described above with reference to FIGS. 1 to 4. Alternatively, the end tool 5100 of the surgical instrument 5000 may correspond to the end tool 3100 described above with reference to FIGS. 14 to 18. Alternatively, the end tool 5100 of the surgical instrument 5000 may correspond to the end tool 1100 or the end tool 3100, from which at least some components are modified or omitted. The end tool 5100 of the surgical instrument 5000 may yaw-rotate in the +direction around the yaw rotation shaft (Z-axis).

At this time, while the end tool 5100 is yaw-rotated in the +direction around the yaw rotation shaft (Z-axis), the first jaw 5101 and the second jaw 5102 of the end tool 5100 may perform an actuation motion. Also, the end tool 5100 of the surgical instrument 5000 may yaw-rotate in the −direction around the yaw rotation shaft (Z-axis).

At this time, while the end tool 5100 is yaw-rotated in the −direction around the yaw rotation shaft (Z-axis), the first jaw 5101 and the second jaw 5102 of the end tool 5100 may perform an actuation motion. Here, the rotation angle of the end tool 5100 may be set variously depending on the ratio of pulleys.

On the other hand, the end tool 5100 of the surgical instrument 5000 may roll-rotate around the roll rotation shaft (X-axis) when the end tool 5100 did not rotate around the yaw rotation shaft (Z-axis), after yaw-rotating in the +direction, or after yaw-rotating in the −direction.

At this time, the end tool 5100 may roll-rotate with the first jaw 5101 and the second jaw 5102 spread apart from each other or may roll-rotate while the first jaw 5101 and the second jaw 5102 are performing an actuation motion. Here, the end tool 5100 is mechanically connected to a motor pack of a power generation portion and may rotate together with the motor pack.

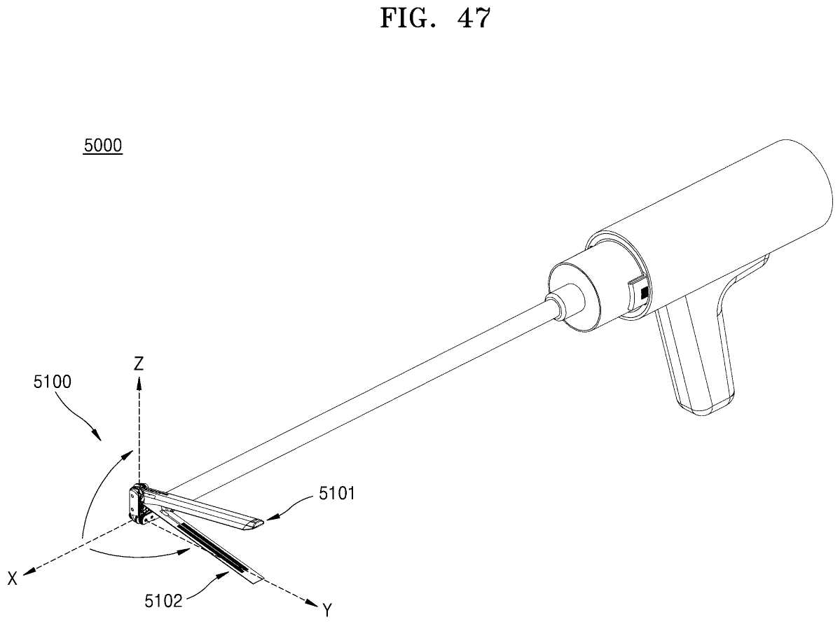

When the motor pack of the power generation portion rolls, the power transmission portion connected to the power generation portion, the connection portion connected to the power transmission portion, and the end tool 5100 formed on one side of the connection portion may rotate simultaneously. FIGS. 47 to 51 are diagrams showing states that a surgical instrument according to the other embodiment of the present disclosure has pitch-rotated and yaw-rotated.

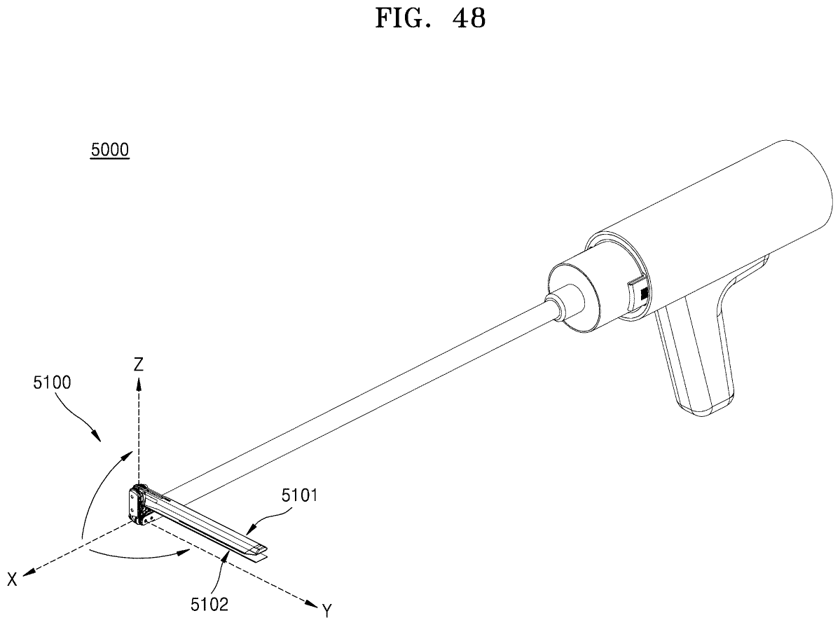

In detail, FIG. 47 is a diagram showing a state in which jaws have pitch-rotated by −90° and yaw-rotated by +90°, and FIG. 48 is a diagram showing a process of performing an actuation motion in the state in which jaws have pitch-rotated by −90° and yaw-rotated by +90°.

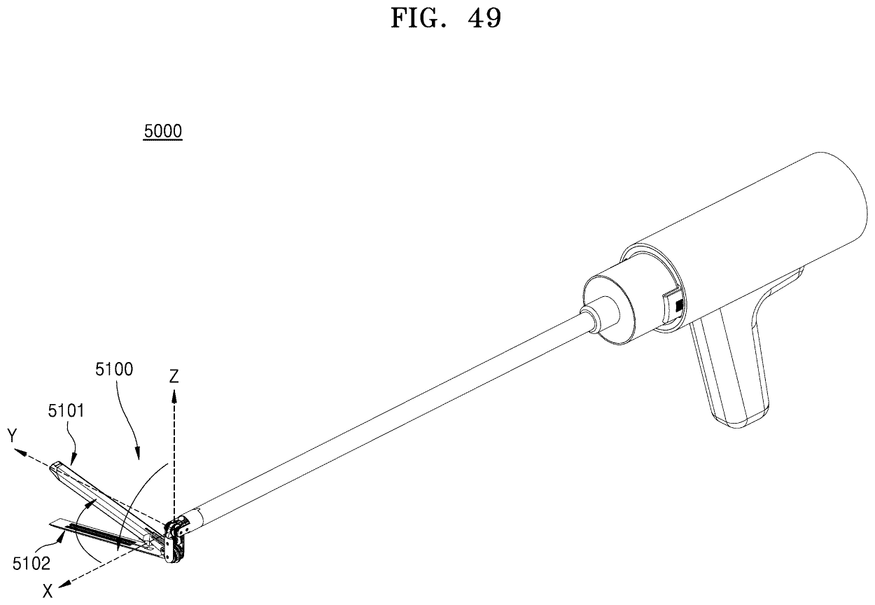

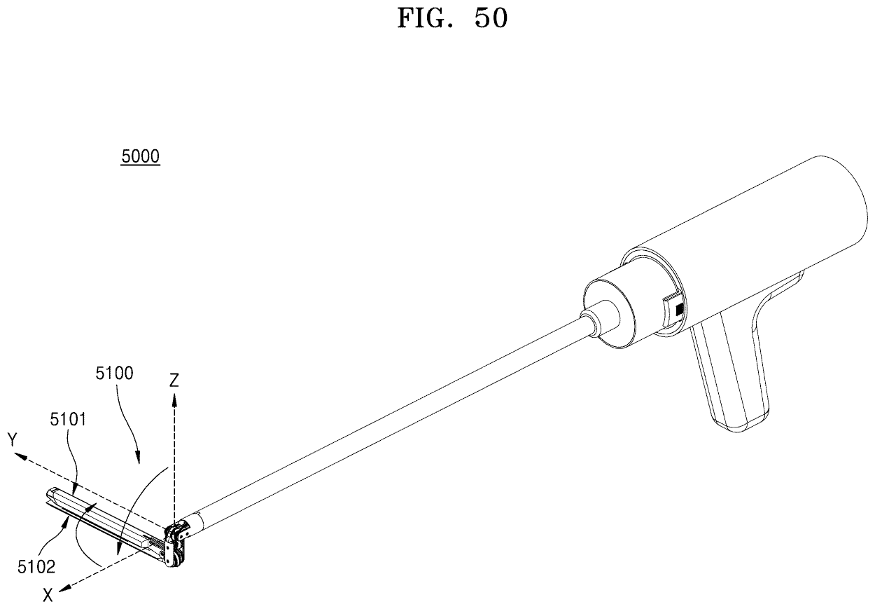

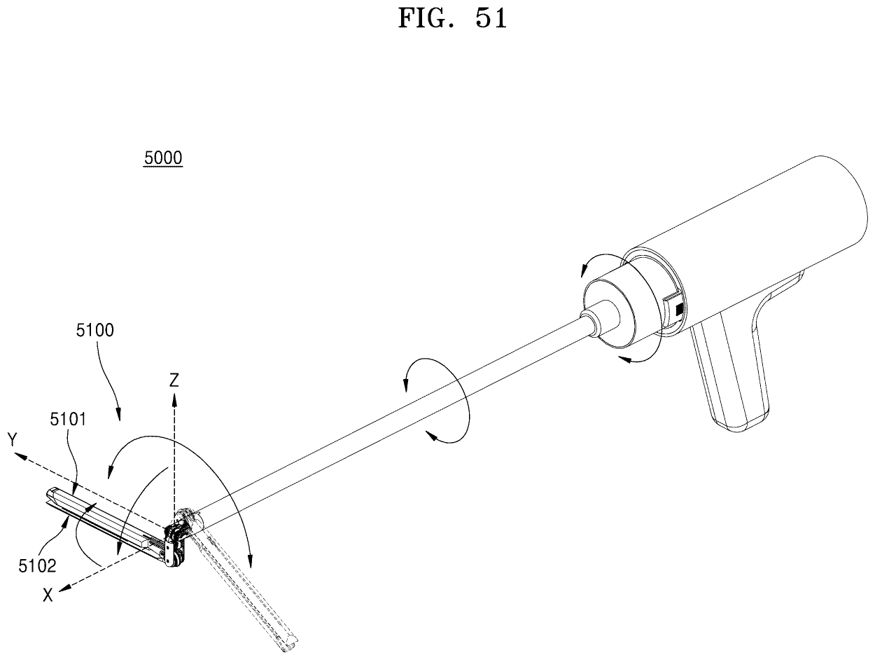

FIG. 49 is a diagram showing a state in which jaws have pitch-rotated by +90° and yaw-rotated by −90°, FIG. 50 is a diagram showing a process of performing an actuation motion in the state in which jaws have pitch-rotated by +90° and yaw-rotated by −90°, and FIG. 51 is a diagram showing a state in which a roll motion is performed after jaws have pitch-rotated and yaw-rotated. Referring to FIGS. 47 to 51, the surgical instrument 5000 according to the present disclosure may include the end tool 5100 including the first jaw 5101 and the second jaw 5102.

Here, the end tool 5100 of the surgical instrument 5000 may correspond to the end tool 1100 described above with reference to FIGS. 1 to 4. Alternatively, the end tool 5100 of the surgical instrument 5000 may correspond to the end tool 3100 described above with reference to FIGS. 14 to 18. Alternatively, the end tool 5100 of the surgical instrument 5000 may correspond to the end tool 1100 or the end tool 3100, from which at least some components are modified or omitted. The end tool 5100 of the surgical instrument 5000 may yaw-rotate around the yaw rotation shaft (Z-axis) and pitch-rotate around the pitch rotation shaft (Y-axis).

In other words, the end tool 5100 of the surgical instrument 5000 may simultaneously perform yaw rotation and pitch rotation. At this time, while the end tool 5100 is yaw-rotated and pitch-rotated, the first jaw 5101 and the second jaw 5102 of the end tool 5100 may perform an actuation motion. Here, the rotation angle of the end tool 5100 may be set variously depending on the ratio of pulleys.

Meanwhile, the end tool 5100 of the surgical instrument 5000 may roll-rotate around the roll rotation shaft (X-axis) after being pitch-rotated and yaw-rotated.

At this time, the end tool 5100 may roll-rotate with the first jaw 5101 and the second jaw 5102 spread apart from each other or may roll-rotate while the first jaw 5101 and the second jaw 5102 are performing an actuation motion. Here, the end tool 5100 may rotate together with a motor pack.

When the motor pack of the power generation portion rolls, the power transmission portion connected to the power generation portion, the connection portion connected to the power transmission portion, and the end tool 5100 formed on one side of the connection portion may rotate simultaneously. As described above, since the surgical instrument 1000 or 3000 according to the present disclosure is configured such that the motor pack 1510 or 3510, the power transmission portion 1300 or 3300, and the end tool 1100 or 3100 may roll together at the same time, wires may not be twisted inside the surgical instrument 1000 or 3000.

This has the technical significance of enabling unlimited roll-rotation of the surgical instrument 1000 or 3000. For example, in a conventional surgical instrument, pulleys inside a power transmission portion do not roll-rotate and only a connection portion and an end tool roll-rotate, and thus wires connecting the end tool and the power transmission portion are twisted inside the connection portion.

In this case, when the end tool and the connection portion continue to roll-rotate, the wires will eventually break or be damaged. In contrast, in the surgical instrument 1000 or 3000 according to the present disclosure, the end tool 1100 or 3100, the connection portion 1400 or 3400, and pulleys 1320, 1330, 3320, 3330, and 3340 inside the power transmission portion 1300 or 3300 may roll-rotate together.

In other words, it may be said that the start points and the end points of wires 1361, 1362, 1363, 1364, 3361, 3362, 3363, 3364, 3365, and 3366 connecting the end tool 1100 or 3100 and the pulleys 1320, 1330, 3320, 3330, and 3340 of the power transmission portion 3300 roll-rotate together. As a result, the wires 1361, 1362, 1363, 1364, 3361, 3362, 3363, 3364, 3365, and 3366 are not twisted inside the connection portion 1400 or 3400. As such, the present disclosure has been described with reference to one embodiment shown in the drawings, but it will be understood that this is merely exemplary, and those of ordinary skill in the art will understand that various modifications and variations of the embodiments are possible therefrom.

Accordingly, the true technical protection scope of the present disclosure should be defined by the technical idea of the appended claims. Certain implementations described in embodiments are merely examples and do not limit the scope of the embodiment in any way.

For the sake of brevity of the specification, descriptions of conventional electronic components, control systems, software, and other functional aspects of the systems may be omitted. Additionally, the connections or connections of lines between the components shown in the drawing exemplify functional connections and/or physical or circuit connections, which can be represented as replaceable or additional various functional connections, physical connections, or circuit connections in real devices. In addition, it may not be an essential component for the application of the present disclosure unless there is a specific mention such as “essential” or “important”. The use of the term “above” and similar indicative terms in the specification of the embodiment (especially in the scope of the patent claim) may correspond to both singular and plural.

In addition, if the range is described in the embodiment, it includes an invention to which individual values belonging to the range are applied (if there is no contrary description), and each individual value constituting the range is described in the detailed description. Lastly, unless the order of the steps constituting the method according to the embodiment is clearly stated or there is no description to the contrary, the steps may be performed in an appropriate order. The embodiments are not necessarily limited by the order of description of the above steps. The use of all examples or exemplary terms (e.g., etc.) in an embodiment is simply for describing the embodiment in detail, and the scope of the embodiment is not limited by the above examples or exemplary terms unless limited by the scope of the patent claim. In addition, those skilled in the art can see that various modifications, combinations and changes can be configured according to design conditions and factors within the scope of the appended claims or their equivalents. The present disclosure may provide a surgical instrument capable of axial rotation (roll) without limitation in rotation angle.

However, these effects are exemplary, and the effects of the present disclosure are not limited thereto.

It should be understood that embodiments described herein should be considered in a descriptive sense only and not for purposes of limitation.

Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the concept and scope as defined by the following claims.

What is claimed is:

1. A surgical instrument comprising:
an end tool comprising a first jaw and a second jaw, each rotatably formed and each configured to rotate in at least one direction;
a manipulation portion configured to receive a signal input by a user to control an operation of the end tool; and
a power generation portion disposed to be at least partially accommodated in a housing of the manipulation portion and comprising a motor pack having at least one motor configured to generate power to drive the end tool based on a signal input to the manipulation portion, and a roll drive motor configured to generate driving force to roll-rotate the motor pack.

2. The surgical instrument of claim 1, wherein the motor pack is configured to rotate independently of a movement of the housing.

3. The surgical instrument of claim 1, wherein the roll drive motor is provided in the motor pack.

4. The surgical instrument of claim 3, wherein the power generation portion comprises:
a first gear formed to have a circular shape and having gear teeth formed on an inner circumferential surface thereof; and
a second gear disposed on one side of the roll drive motor and engaged with the first gear.

5. The surgical instrument of claim 4,
wherein the first gear is fixed to the inner circumferential surface of the housing, and
wherein the second gear is formed to move along the inner circumferential surface of the first gear while being rotated by driving the roll drive motor.

6. The surgical instrument of claim 1, wherein the end tool is connected to the motor pack and is configured to roll-rotate together with the motor pack.

7. The surgical instrument of claim 1, further comprising a power transmission portion comprising at least one wire transmitting power generated by the power generation portion to the end tool.

8. The surgical instrument of claim 7, wherein the power transmission portion is connected to the motor pack and is configured to roll-rotate together with the motor pack.

9. The surgical instrument of claim 8, further comprising a connection portion disposed between the power transmission portion and the end tool and accommodating the wire therein.

10. The surgical instrument of claim 9, wherein the connection portion is connected to the power transmission portion and is configured to roll-rotate together with the power transmission portion by roll rotation of the power transmission portion.

11. The surgical instrument of claim 7, wherein the power generation portion further comprises a pulley coupling plate coupled to the power transmission portion, and the pulley coupling plate is configured to rotate together with the motor pack by driving of the roll drive motor.

12. The surgical instrument of claim 11, wherein the power transmission portion is configured to roll-rotate together with the pulley coupling plate while being coupled to the pulley coupling plate.

13. The surgical instrument of claim 1, wherein the motor pack further comprises:
a yaw drive motor configured to generate power to yaw-rotate the end tool; and
a pitch drive motor configured to generate power to pitch-rotate the end tool.

14. The surgical instrument of claim 13,
wherein the power generation portion further comprises a base plate connected to the roll drive motor, the yaw drive motor, and the pitch drive motor, and
wherein the base plate is rotated by driving the roll drive motor, and the roll drive motor, the yaw drive motor, and the pitch drive motor rotate simultaneously by the rotation of the base plate.

15. The surgical instrument of claim 13,
wherein the end tool further comprises a moving member configured to move along a lengthwise direction of the end tool, and
wherein the motor pack further comprises a firing drive motor configured to generate power to linearly move the moving member.

16. The surgical instrument of claim 15,
wherein the power generation portion further comprises a base plate connected to the roll drive motor, the yaw drive motor, the pitch drive motor, and the firing drive motor, and
wherein the base plate is rotated by driving the roll drive motor, and the roll drive motor, the yaw drive motor, the pitch drive motor, and the firing drive motor rotate simultaneously by the rotation of the base plate.

17. The surgical instrument of claim 14, wherein the power generation portion further comprises:
a bearing plate connected to the base plate; and
first bearings arranged to cover an outer circumferential surface of the bearing plate.

18. The surgical instrument of claim 1, further comprising a circuit unit disposed on one side of the motor pack and configured to control driving of the motor pack.

19. The surgical instrument of claim 18, wherein the circuit unit is connected to the motor pack and is configured to roll-rotate together with the motor pack.

20. The surgical instrument of claim 18, further comprising a slip ring disposed on one side of the circuit unit and electrically connecting the motor pack and the circuit unit to each other.
